SEQUENCE LISTING (13 sequences)

SEQ ID NO: 1 - protein, 6 aa, artificial
  Synthetic peptide
  UNSURE (2)..(2): Xaa at position 2 is phosphorylated threonine
VXPEER

SEQ ID NO: 2 - protein, 4 aa, unknown
  RNA binding domain of stem-loop-binding protein
TPNK

SEQ ID NO: 3 - protein, 10 aa, artificial
  Peptide moiety
ERHLSKMQQC

SEQ ID NO: 4 - protein, 26 aa, artificial
  Peptide moiety
ERHLSKMQQNGYENPTYKFFEQMQNC

SEQ ID NO: 5 - protein, 11 aa, artificial
  Synthetic internalization sequence
YARAAARQARA

SEQ ID NO: 6 - protein, 36 aa, artificial
  Peptide moiety
ERHLSKMQQNGYENPTYKFFEQMQNYARAAARQARA

SEQ ID NO: 7 - protein, 20 aa, artificial
  Peptide moiety
ERHLSKMQQYARAAARQARA

SEQ ID NO: 8 - protein, 6 aa, artificial
  Peptide moiety
CVDAAV

SEQ ID NO: 9 - protein, 16 aa, artificial
  Peptide moiety
YARAAARQARAVDAAV

SEQ ID NO: 10 - protein, 30 aa, artificial
  Peptide moiety
YARAAARQARAKKKQYTSIHHGVVEVDAAV

SEQ ID NO: 11 - protein, 20 aa, artificial
  Peptide moiety
CKKKQYTSIHHGVVEVDAAV

SEQ ID NO: 12 - protein, 21 aa, unknown
  Fragment of amyloid precursor protein
  UNSURE (10)..(10): Xaa at position 10 is phosphorylated threonine
GVVEVDAAVXPEERHLSKMQQ

SEQ ID NO: 13 - protein, 14 aa, artificial
  Synthetic peptide
  UNSURE (7)..(7): Xaa at position 7 is phosphorylated threonine
EVDAAVXPEERHLS

(19) United States
(12) Patent Application Publication
(10) Pub. No.: US 2008/0058276 A1
(43) Pub. Date: Mar. 6, 2008

(54) ALZHEIMER'S DISEASE THERAPEUTICS BASED ON PIN-1 CATALYZED CONFORMATIONAL CHANGES IN PHOSPHORYLATED AMYLOID PRECURSOR PROTEIN

(75) Inventors: Kun Ping LU, Newton, MA (US); Linda K. NICHOLSON, Ithaca, NY (US); Lucia PASTORINO, Boston, MA (US); Xiao Zhen ZHOU, Newton, MA (US); Soumya DE, Ithaca, NY (US)

Correspondence Address: NIXON PEABODY LLP - PATENT GROUP, CLINTON SQUARE, P.O. BOX 31051, ROCHESTER, NY, 14603-1051, US

(73) Assignees: CORNELL RESEARCH FOUNDATION, INC., Ithaca, NY (US); BETH ISRAEL DEACONESS MEDICAL CENTER, Boston, MA (US)

(21) Appl. No.: 11/623,672

(22) Filed: Jan. 16, 2007

Related U.S. Application Data
(60) Provisional application No. 60/759,203, filed on Jan. 13, 2006.

(51) Int. Cl. A61K 31/70; A61P 25/28; C12N 5/06; C12Q 1/02; G01N 33/00

(52) U.S. Cl. 514/44; 435/29; 435/375; 436/86

(57) ABSTRACT

The present invention is directed to inhibiting amyloidogenic processing of amyloid precursor protein, and/or inhibiting production of amyloid beta peptides. These methods can involve accelerating cis/trans isomerization of amyloid precursor protein at a phosphorylated serine/threonine-proline motif and/or contacting a cell with a compound that mimics the cis conformation of a phosphorylated serine/threonine-proline motif of an amyloid precursor protein. The present invention also relates to treating and/or preventing in a subject a degenerative neurological disease characterized by amyloidogenic processing of amyloid precursor protein and/or overproduction of amyloid beta peptide. This method involves administering an agent that accelerates cis/trans isomerization of amyloid precursor protein at a phosphorylated serine/threonine-proline motif and/or inhibits production of amyloid β peptides. Methods of screening for therapeutic agents effective in treating and/or preventing such diseases, methods of screening for biological molecules involved in the amyloidogenic pathway, and compounds that mimic the cis conformation of a phosphorylated serine/threonine-proline motif of an amyloid precursor protein are also disclosed.

This application claims the benefit of U.S. Provisional Patent Application Ser. No. 60/759,203, filed Jan. 13, 2006, which is hereby incorporated by reference in its entirety.

The present invention was made, at least in part, with funding received from the National Institutes of Health, grant numbers GM058556, AG0178870, and AG022082; and the National Science Foundation, grant number MCB-0212597. The U.S. Government may have certain rights in this invention.

### FIELD OF THE INVENTION

This invention is directed generally to compounds and methods for inhibiting amyloidogenic processing of amyloid precursor protein and Aβ peptide production.

### BACKGROUND OF THE INVENTION

Alzheimer's disease (“AD”) is a major age-dependent neurodegeneration, displaying two pathological hallmarks: senile plaques, which correlate with overproduction of amyloid-β (“Aβ”) peptides, and neurofibrillary tangles (“NFTs”), which arise from hyperphosphorylation/dysfunction of the microtubule-associated protein tau (Selkoe, “The Cell Biology of β-Amyloid Precursor Protein and Presenilin in Alzheimer's Disease,” Trends Cell Biol 8(11):447-53 (1998); Hardy & Selkoe, “The Amyloid Hypothesis of Alzheimer's Disease: Progress and Problems on the Road to Therapeutics,” Science 297(5580):353-6 (2002); Spillantini & Goedert, “Tau Protein Pathology in Neurodegenerative Diseases,” Trends Neurosci 21(10):428-33 (1998); Lee, “Tauists and β-Aptists United—Well Almost!,” Science 293(5534):1446-7 (2001); Wolfe, “Therapeutic Strategies for Alzheimer's Disease,” Nat Rev Drug Discov 1(11):859-66 (2002); Wong et al., “Genetically Engineered Mouse Models of Neurodegenerative Diseases,” Nat Neurosci 5(7):633-9 (2002); Spires & Hyman, “Neuronal Structure Is Altered by Amyloid Plaques,” Rev Neurosci 15(4):267-78 (2004); Mattson, “Pathways Towards and Away from Alzheimer's Disease,” Nature 430(7000):631-9 (2004); Goldgaber et al., “Characterization and Chromosomal Localization of a cDNA Encoding Brain Amyloid of Alzheimer's Disease,” Science 235(4791):877-80 (1987); Tanzi et al., “Amyloid β Protein Gene: cDNA, mRNA Distribution, and Genetic Linkage Near the Alzheimer Locus,” Science 235(4791):880-4 (1987); Kang et al., “The Precursor of Alzheimer's Disease Amyloid A4 Protein Resembles a Cell-surface Receptor,” Nature 325(6106):733-6 (1987); Goedert et al., “Cloning and Sequencing of the cDNA Encoding a Core Protein of the Paired Helical Filament of Alzheimer Disease: Identification as the Microtubule-associated Protein Tau,” Proc Nat'l Acad Sci USA 85(11):4051-5 (1988); Wischik et al., “Structural Characterization of the Core of the Paired Helical Filament of Alzheimer Disease,” Proc Nat'l Acad Sci USA 85(13):4884-8 (1988); Kondo et al., “The Carboxyl Third of Tau Is Tightly Bound to Paired Helical Filaments,” Neuron 1(9):827-34 (1988); Bancher et al., “Accumulation of Abnormally Phosphorylated Tau Precedes the Formation of Neurofibrillary Tangles in Alzheimer's Disease,” Brain Res 477(1-2):90-9 (1989); Lee et al., “A68: A Major Subunit of Paired Helical Filaments and Derivatized Forms of Normal Tau,” Science 251(4994):675-8 (1991); Goedert et al., “Tau Proteins of Alzheimer Paired Helical Filaments: Abnormal Phosphorylation of All Six Brain Isoforms,” Neuron 8(1):159-68 (1992); Greenberg et al., “Hydrofluoric Acid-treated Tau PHF Proteins Display the Same Biochemical Properties as Normal Tau,” J Biol Chem 267(1):564-9 (1992); Lee, “Disruption of the Cytoskeleton in Alzheimer's Disease,” Curr Opin Neurobiol 5(5):663-8 (1995); Mandelkow et al., “On the Structure of Microtubules, Tau, and Paired Helical Filaments,” Neurobiol Aging 16(3):347-54 (1995)).

β-Amyloid peptides are insoluble peptides of approximately 4 kDa generated from amyloid precursor protein (“APP”), a transmembrane protein that contains a small intracellular COOH-terminal domain (Hardy & Selkoe, “The Amyloid Hypothesis of Alzheimer's Disease: Progress and Problems on the Road to Therapeutics,” Science 297(5580):353-6 (2002); Goldgaber et al., “Characterization and Chromosomal Localization of a cDNA Encoding Brain Amyloid of Alzheimer's Disease,” Science 235(4791):877-80 (1987); Tanzi et al., “Amyloid β Protein Gene: cDNA, mRNA Distribution, and Genetic Linkage Near the Alzheimer Locus,” Science 235(4791):880-4 (1987); Kang et al., “The Precursor of Alzheimer's Disease Amyloid A4 Protein Resembles a Cell-surface Receptor,” Nature 325(6106):733-6 (1987); Nunan & Small, “Proteolytic Processing of the Amyloid-β Protein Precursor of Alzheimer's Disease,” Essays Biochem 38:37-49 (2002); De Strooper & Annaert, “Proteolytic Processing and Cell Biological Functions of the Amyloid Precursor Protein,” J Cell Sci 113(Pt 11):1857-70 (2000)). As shown in FIG. 1, APP is processed through the so-called amyloidogenic or non-amyloidogenic pathways (Nunan & Small, “Proteolytic Processing of the Amyloid-β Protein Precursor of Alzheimer's Disease,” Essays Biochem 38:37-49 (2002); Selkoe et al., “The Role of APP Processing and Trafficking Pathways in the Formation of Amyloid β-Protein,” Ann NY Acad Sci 777:57-64 (1996)).

The amyloidogenic pathway involves the activity of β- and γ-secretases and requires the internalization of APP to the endosomes and subsequent structures (Estus et al., “Potentially Amyloidogenic, Carboxyl-terminal Derivatives of the Amyloid Protein Precursor,” Science 255(5045):726-8 (1992); Golde et al., “Processing of the Amyloid Protein Precursor to Potentially Amyloidogenic Derivatives,” Science 255(5045):728-30 (1992); Haass et al., “Targeting of Cell-surface β-Amyloid Precursor Protein to Lysosomes: Alternative Processing into Amyloid-bearing Fragments,” Nature 357(6378):500-3 (1992); Haass et al., “Amyloid β-Peptide Is Produced by Cultured Cells During Normal Metabolism,” Nature 359(6393):322-5 (1992); Shoji et al., “Production of the Alzheimer Amyloid Beta Protein by Normal Proteolytic Processing,” Science 258(5079):126-9 (1992); De Strooper et al., “Study of the Synthesis and Secretion of Normal and Artificial Mutants of Murine Amyloid Precursor Protein (APP): Cleavage of APP Occurs in a Late Compartment of the Default Secretion Pathway,” J Cell Biol 121(2):295-304 (1993); Koo & Squazzo, “Evidence that Production and Release of Amyloid β-Protein Involves the Endocytic Pathway,” J Biol Chem 269(26):17386-9 (1994); Koo et al., “Trafficking of Cell-surface Amyloid β-Protein Precursor I. Secretion, Endocytosis and Recycling as Detected by Labeled Monoclonal Antibody,” J Cell Sci 109(Pt 5):991-8 (1996); Perez et al., “Mutagenesis Identifies New Signals for β-Amyloid Precursor Protein Endocytosis, Turnover, and the Generation of Secreted Fragments, Including Aβ42,” J Biol Chem 274(27):18851-6 (1999)). As shown in FIG. 1 (left side), β-secretase (“BACE”) cuts APP at the beginning of the sequence of Aβ, generating an extracellular soluble fragment called βAPPs and an intracellular COOH-terminal fragment called βCTF (Yan et al., “Membrane-anchored Aspartyl Protease with Alzheimer's Disease β-Secretase Activity,” Nature 402(6761):533-7 (1999); Vassar et al., “β-Secretase Cleavage of Alzheimer's Amyloid Precursor Protein by the Transmembrane Aspartic Protease BACE,” Science 286(5440):735-41 (1999); Hussain et al., “Identification of a Novel Aspartic Protease (Asp 2) as β-Secretase,” Mol Cell Neurosci 14(6):419-27 (1999); Cai et al., “BACE1 Is the Major β-Secretase for Generation of Aβ Peptides by Neurons,” Nat Neurosci 4(3):233-4 (2001)). Subsequently, γ-secretase cuts CTF at residues 40, 42, or 43 of the Aβ sequence, generating intact Aβ species (Wolfe et al., “Two Transmembrane Aspartates in Presenilin-1 Required for Presenilin Endoproteolysis and γ-Secretase Activity,” Nature 398(6727):513-7 (1999)).

As shown in FIG. 1 (right side), the non-amyloidogenic pathway involves the activity of α-secretase at the plasma membrane level (Esch et al., “Cleavage of Amyloid Beta Peptide During Constitutive Processing of Its Precursor,” Science 248(4959):1122-4 (1990); Sisodia et al., “Evidence that β-Amyloid Protein in Alzheimer's Disease Is not Derived by Normal Processing,” Science 248(4954):492-5 (1990); Parvathy et al., “Cleavage of Alzheimer's Amyloid Precursor Protein by α-Secretase Occurs at the Surface of Neuronal Cells,” Biochemistry 38(30):9728-34 (1999); Buxbaum et al., “Evidence that Tumor Necrosis Factor a Converting Enzyme Is Involved in Regulated α-Secretase Cleavage of the Alzheimer Amyloid Protein Precursor,” J Biol Chem 273(43):27765-7 (1998)). α-Secretase cuts within the sequence of Aβ, generating αAPPs without resulting in release of intact Aβ or any amyloidogenic products (Esch et al., “Cleavage of Amyloid Beta Peptide During Constitutive Processing of Its Precursor,” Science 248(4959):1122-4 (1990); Sisodia et al., “Evidence that β-Amyloid Protein in Alzheimer's Disease Is not Derived by Normal Processing,” Science 248(4954):492-5 (1990)).

Mutations in APP and presenilin, as well as other proteins, affect the regulation of Aβ production and have been well documented in AD (Goate et al., “Segregation of a Missense Mutation in the Amyloid Precursor Protein Gene with Familial Alzheimer's Disease,” Nature 349(6311):704-6 (1991); Mullan et al., “A Pathogenic Mutation for Probable Alzheimer's Disease in the APP Gene at the N-terminus of β-Amyloid,” Nat Genet 1(5):345-7 (1992); Hendriks et al., “Presenile Dementia and Cerebral Haemorrhage Linked to a Mutation at Codon 692 of the β-Amyloid Precursor Protein Gene,” Nat Genet 1(3):218-21 (1992); Corder et al., “Gene Dose of Apolipoprotein E Type 4 Allele and the Risk of Alzheimer's Disease in Late Onset Families,” Science 261(5123):921-3 (1993); Levy-Lahad et al., “Candidate Gene for the Chromosome 1 Familial Alzheimer's Disease Locus,” Science 269(5226):973-7 (1995); Sherrington et al., “Cloning of a Gene Bearing Missense Mutations in Early-onset Familial Alzheimer's Disease,” Nature 375(6534):754-60 (1995); Scheuner et al., “Secreted Amyloid β-Protein Similar to That in the Senile Plaques of Alzheimer's Disease Is Increased in Vivo by the Presenilin 1 and 2 and APP Mutations Linked to Familial Alzheimer's Disease,” Nat Med 2(8):864-70 (1996); Duff et al., “Increased Amyloid-042(43) in Brains of Mice Expressing Mutant Presenilin 1,” Nature 383(6602):710-3 (1996)). In transgenic mice, overexpression of mutant APP or presenilin results in increased Aβ, senile plaques, and memory loss (Wong et al., “Genetically Engineered Mouse Models of Neurodegenerative Diseases,” Nat Neurosci 5(7):633-9 (2002); Duff et al., “Increased Amyloid-β42(43) in Brains of Mice Expressing Mutant Presenilin 1,” Nature 383(6602):710-3 (1996); Games et al., “Alzheimer-type Neuropathology in Transgenic Mice Overexpressing V717F β-Amyloid Precursor Protein,” Nature 373(6514):523-7 (1995); Hsiao et al., “Correlative Memory Deficits, Aβ Elevation, and Amyloid Plaques in Transgenic Mice,” Science 274(5284):99-102 (1996); Borchelt et al., “Familial Alzheimer's Disease-linked Presenilin 1 Variants Elevate Aβ1-42/1-40 Ratio in Vitro and in Vivo,” Neuron 17(5):1005-13 (1996); Borchelt et al., “Accelerated Amyloid Deposition in the Brains of Transgenic Mice Coexpressing Mutant Presenilin 1 and Amyloid Precursor Proteins,” Neuron 19(4):939-45 (1997); Chen et al., “A Learning Deficit Related to Age and β-Amyloid Plaques in a Mouse Model of Alzheimer's Disease,” Nature 408(6815):975-9 (2000); Trinchese et al., “Progressive Age-related Development of Alzheimer-like Pathology in APP/PSI Mice,” Ann Neurol 55(6):801-14 (2004)). Therefore, it is important to understand the events that determine whether APP enters a normal or amyloidogenic processing pathway.

Increased phosphorylation of tau on serine or threonine residues, especially those preceding a proline residue (“pSer/Thr-Pro”) precedes tangle formation and neurodegeneration (Vincent et al., “The Cell Cycle and Human Neurodegenerative Disease,” Prog Cell Cycle Res 5:31-41 (2003); Lee & Tsai, “Cdk5: One of the Links Between Senile Plaques and Neurofibrillary Tangles?,” J Alzheimers Dis 5(2): 127-37 (2003); Zhu et al., “Oxidative Stress Signalling in Alzheimer's Disease,” Brain Res 1000(1-2):32-9 (2004); Lu, “Pinning Down Cell Signaling, Cancer and Alzheimer's Disease,” Trends Biochem Sci 29:200-209 (2004)). Recently, increased phosphorylation of APP on Thr-Pro has also been reported in AD brains and implicated in regulating APP processing and Aβ production (Lee & Tsai, “Cdk5: One of the Links Between Senile Plaques and Neurofibrillary Tangles?,” J Alzheimers Dis 5(2):127-37 (2003); Phiel et al., “GSK-3α Regulates Production of Alzheimer's Disease Amyloid-β Peptides,” Nature 423(6938):435-9 (2003)). Hence, the pSer/Thr-Pro motif appears to play a central role in the development of senile plaques and neurofibrillary tangles, characteristic of AD.

Pin1 and its homologues are the only enzymes known so far that can specifically isomerize pSer/Thr-Pro bonds with high efficiency (Ranganathan et al., “Structural and Functional Analysis of the Mitotic Rotamase Pin1 Suggests Substrate Recognition Is Phosphorylation Dependent,” Cell 89:875-886 (1997); Yaffe et al., “Sequence-specific and Phosphorylation-dependent Proline Isomerization: A Potential Mitotic Regulatory Mechanism,” Science 278:1957-1960 (1997); Lu et al., “A Human Peptidyl-prolyl Isomerase Essential for Regulation of Mitosis,” Nature 380(6574):544-7 (1996), which are hereby incorporated by reference in their entirety). As shown in FIG. 2, the WW domain of Pin1 binds to specific pSer/Thr-Pro-motifs, targeting the Pin1 catalytic domain close to its substrates, where the PPIase domain catalyzes isomerization of specific pSer/Thr-Pro motifs and induces conformational changes in proteins (Zhou et al., “Pin1-dependent Prolyl Isomerization Regulates Dephosphorylation of Cdc25C and Tau Proteins,” Mol Cell 6:873-883 (2000); Lu et al., “The Prolyl Isomerase Pin1 Restores the Function of Alzheimer-associated Phosphorylated Tau Protein,” Nature 399:784-788 (1999); Lu et al., “Pinning Down the Proline-directed Phosphorylation Signaling,” Trends Cell Biol 12:164-172 (2002); Shen et al., “The Essential Mitotic Peptidyl-prolyl Isomerase Pin1 Binds and Regulates Mitosis-specific Phosphoproteins,” Genes Dev 12:706-720 (1998); Lu et al., “A Function of WW Domains as Phosphoserine- or Phosphothreonine-binding Modules,” Science 283:1325-1328 (1999); Wulf et al., “Pin1 Is Overexpressed in Breast Cancer and Potentiates the Transcriptional Activity of Phosphorylated c-Jun Towards the Cyclin D1 Gene,” EMBO J 20:3459-3472 (2001); Ryo et al., “Pin1 Regulates Turnover and Subcellular Localization of β-Catenin by Inhibiting Its Interaction with APC,” Nature Cell Biol 3:793-801 (2001)).

Pin1 has been proposed to regulate protein function by accelerating conformational changes (Lu, “Pinning Down Cell Signaling, Cancer and Alzheimer's Disease,” TiBS 29:200-209 (2004); Lu et al., “The Prolyl Isomerase Pin1 Restores the Function of Alzheimer-associated Phosphorylated Tau Protein,” Nature 399:784-788 (1999); Zhou et al., “Pin1-dependent Prolyl Isomerization Regulates Dephosphorylation of Cdc25C and Tau Proteins,” Mol Cell 6:873-883 (2000); Stukenberg & Kirschner, “Pin1 Acts Catalytically to Promote a Conformational Change in Cdc25,” Mol Cell 7(5): 1071-83 (2001)), but such activity had not previously been visualized and the biological and pathological significance of Pin1 substrate conformations had not been known (Lu, “Pinning Down Cell Signaling, Cancer and Alzheimer's Disease,” TiBS 29:200-209 (2004)). Pin1 is downregulated and/or inhibited by oxidation in AD neurons, Pin1 knockout causes tauopathy and neurodegeneration (Lu et al., “The Prolyl Isomerase Pin1 Restores the Function of Alzheimer-associated Phosphorylated Tau Protein,” Nature 399:784-788 (1999); Zhou et al., “Pin1-dependent Prolyl Isomerization Regulates Dephosphorylation of Cdc25C and Tau Proteins,” Mol Cell 6:873-883 (2000); Liou et al., “Role of the Prolyl Isomerase Pin1 in Protecting Against Age-dependent Neurodegeneration,” Nature 424:556-561 (2003); Sultana et al., “Oxidative Modification and Down-regulation of Pin1 in Alzheimer's Disease Hippocampus: A Redox Proteomics Analysis,” Neurobiol Aging 27(7):918-25 (2006 (Epub 2005))) and Pin1 promoter polymorphisms appear to be associated with reduced Pin1 levels and increased risk for late-onset AD (Segat et al., “Pin1 Promoter Polymorphisms are Associated with Alzheimer's Disease,” Neurobiol Aging 28(1):69-74 (2007 (Epub 2005)); Wijsman et al., “Evidence for a Novel Late-onset Alzheimer Disease Locus on Chromosome 19p13.2,” Am J Hum Genet 75(3):398-409 (2004)). However, the role of Pin1 in APP processing and Aβ production, and the biological and pathological significance of cis and trans conformations of APP were unknown.

Thus, there remains a need for defining the biological role of Pin 1 in AD and for identifying methods and agents for regulating APP processing and Aβ production. The present invention is directed to overcoming these and other deficiencies in the art.

### SUMMARY OF THE INVENTION

One aspect of the present invention relates to a method of inhibiting amyloidogenic processing of amyloid precursor protein. This method involves accelerating cis/trans isomerization of the amyloid precursor protein at a phosphorylated serine/threonine-proline motif under conditions effective to inhibit amyloidogenic processing of the amyloid precursor protein.

Another aspect of the present invention relates to a method of inhibiting production of amyloid beta peptides by a cell. This method involves contacting the cell with a compound that mimics the cis conformation of a phosphorylated threonine-proline motif of an amyloid precursor protein under conditions effective to inhibit production of amyloid beta peptides.

Yet another aspect of the present invention relates to a method of inhibiting production of amyloid beta peptides by a cell. This method involves accelerating cis/trans isomerization of amyloid precursor protein at a phosphorylated serine/threonine-proline motif under conditions effective to inhibit production of amyloid beta peptides.

Another aspect of the present invention relates to a method of treating and/or preventing in a subject a degenerative neurological disease characterized by amyloidogenic processing of amyloid precursor protein and/or overproduction of amyloid beta peptide. This method involves administering to the subject an agent that (1) accelerates cis/trans isomerization of amyloid precursor protein at a phosphorylated serine/threonine-proline motif and/or (2) inhibits production of amyloid beta peptides, under conditions effective to treat and/or prevent the disease in the subject.

Yet another aspect of the present invention relates to a method of screening for a therapeutic agent effective in treating and/or preventing in a subject a degenerative neurological disease characterized by amyloidogenic processing of amyloid precursor protein and/or overproduction of amyloid beta peptide. This method involves providing a substrate compound comprising a phosphorylated serine/threonine-proline motif of an amyloid precursor protein, and a candidate compound. The candidate compound is contacted with the substrate compound, and the cis/trans isomerization rate of the phosphorylated serine/threonine-proline motif in the presence of the candidate compound is measured. The cis/trans isomerization rate in the presence of the candidate compound is compared to a reference cis/trans isomerization rate, where acceleration of the cis/trans isomerization rate in the presence of the candidate compound relevant to the reference cis/trans isomerization rate indicates that the candidate compound is a potential therapeutic agent effective in treating and/or preventing in a subject a degenerative neurological disease characterized by amyloidogenic processing of amyloid precursor protein and/or overproduction of amyloid beta peptide.

Another aspect of the present invention relates to a method of screening for a therapeutic agent effective in treating and/or preventing in a subject a degenerative neurological disease characterized by amyloidogenic processing of amyloid precursor protein and/or overproduction of amyloid beta peptide. This method involves providing a temperature sensitive Ess1/Ptf1 mutant yeast cell and contacting the cell with a candidate compound. The cell is cultured at a temperature effective to cause terminal mitotic arrest of the yeast cell due to an absence of Ess1/Ptf1 function, and whether the cell displays a temperature-sensitive phenotype during culturing is evaluated. Compounds that prevent the yeast cell from displaying the temperature-sensitive phenotype are identified as likely therapeutic agents effective in treating and/or preventing in a subject a degenerative neurological disease characterized by amyloidogenic processing of amyloid precursor protein and/or overproduction of amyloid beta peptide.

Another aspect of the present invention relates to a method of screening for biological molecules involved in the amyloidogenic pathway. This method involves (i) contacting an amyloid precursor protein which is phosphorylated at a serine/threonine-proline motif with a neuronal cell lysate and detecting binding of biological molecules from the neuronal cell lysate to the amyloid precursor protein; and (ii) contacting a compound that mimics the cis conformation of a phosphorylated threonine-proline motif of an amyloid precursor protein with a neuronal cell lysate and detecting binding of biological molecules from the neuronal cell lysate to the compound, under conditions essentially the same as in step (i). The binding detected in step (i) is compared with the binding detected in step (ii), where a biological molecule which undergoes greater binding in step (ii) than in step (i) is likely to be involved in the amyloidogenic pathway.

Yet another aspect of the present invention relates to a compound of formula 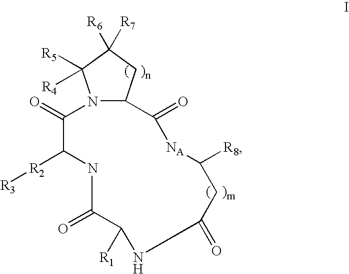
wherein:
R₁ is an amino acid side chain;
R₂ is a glutamic acid-based side chain, an aspartic acid-based side chain, or a moiety of the formula -Ser/Thr-X—Y₍₂₎, where Ser/Thr is a serine amino acid-based side chain or a threonine amino acid-based side chain, X is a negatively charged tetra- or penta-valent moiety selected from the group consisting of —OPO₃²⁻, —PO₃²⁻, —OSO₃²⁻, and —OBO₂²⁻, and Y is independently hydrogen, a blocking group, or absent;
R₃ is absent or a linker between R₂ and N_{A};
R₄ and R₅ are independently hydrogen or C₁₋₃ alkyl;
R₆ and R₇ are independently hydrogen or halogen;
R₈ is —COR where R is a peptide of 0 to approximately 40 amino acid units;
m is 1 or 2;
n is 1, 2, or 3; and
R₁ and/or R₈ are optionally modified to facilitate transport and/or cellular uptake of
the compound and/or attachment of the compound to a substrate; and
   wherein the compound mimics the cis conformation of a phosphorylated serine/threonine-proline motif of an amyloid precursor protein.

Yet another aspect of the present invention relates to a compound of formula 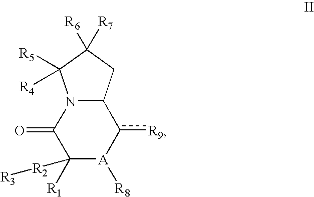
wherein:
R₁ is —H or —NHRₐ where Rₐ is a peptide of 0 to approximately 40 amino acid units;
R₂ is a glutamic acid-based side chain, an aspartic acid-based side chain, or a moiety of the formula -Ser/Thr-X—Y₍₂₎, where Ser/Thr is a serine amino acid-based side chain or a threonine amino acid-based side chain, X is a negatively charged tetra- or penta-valent moiety selected from the group consisting of —OPO₃²⁻, —PO₃²⁻, —OSO₃²⁻, and —OBO₂²⁻, and Y is independently hydrogen, a blocking group, or absent;
R₃ is absent or a linker between R₂ and A;
R₄ and R₅ are independently hydrogen or C₁₋₃ alkyl;
R₆ and R₇ are independently hydrogen or halogen;
R₈ is —H or —CH(CH₂)₂COOHCOR_{b} where R_{b} is a peptide of 0 to approximately 40 amino acid units;
R₉ is a hydrogen bond acceptor;
A is N, O, C, or S;
[symbol] is a single or double bond; and
R₁ and/or R₈ are optionally modified to facilitate transport and/or cellular uptake of the compound and/or attachment of the compound to a substrate; and
   wherein the compound mimics the cis conformation of a phosphorylated serine/threonine-proline motif of an amyloid precursor protein.

The present invention shows that Pin1 has profound effects on APP processing and Aβ production. Pin1 was found to bind to the phosphorylated Thr668-Pro motif in APP and accelerate its isomerization by over 1000 fold, regulating the APP intracellular domain between two conformations, as visualized by NMR. Whereas Pin1 overexpression reduces, its knockout increases Aβ secretion from cell cultures. Pin1 knockout alone or in combination with APP mutant overexpression in mice increases amyloidogenic APP processing and selectively elevates insoluble Aβ42 in brains in an age-dependent manner, with Aβ42 being prominently localized to multivesicular bodies of neurons, as shown in AD before plaque pathology (Takahashi et al., “Intraneuronal Alzheimer Aβ42 Accumulates in Multivesicular Bodies and Is Associated with Synaptic Pathology,” Am J Pathol 161(5): 1869-79 (2002), which is hereby incorporated by reference in its entirety). Thus, Pin1-catalyzed prolyl isomerization is a novel post-phosphorylation signaling mechanism in the regulation of APP processing and Aβ production, and its deregulation may link both tangle and plaque pathologies. These findings provide a new insight into the pathogenesis and treatment of AD. The present invention provides important compounds and methods for protecting against Alzheimer's disease.

### BRIEF DESCRIPTION OF THE DRAWINGS

FIG. 1 is a schematic diagram of amyloid precursor protein (“APP”) processing and Aβ peptide production. APP (top) is composed of an extracellular domain (dark bar), a transmembrane domain (“TM”), and an intracellular domain (“AICD”) that contains a Thr-Pro motif at residues 668-669. APP is processed by various secretases into the non-amyloidogenic or amyloidogenic pathway, with the amyloidogenic pathway leading to the production of Aβ peptides, causing the amyloid plaque pathology. In particular, APP is cleaved by β- or α-secretase to form an N-terminal fragment (βAPPs or αAPPs, respectively) and a C-terminal fragment (βCTF or αCTF, respectively). The C-terminal fragment is further cleaved by γ-secretase to generate C-terminal C59/CTFs, which is imported into the nucleus to activate transcription, and either Aβ peptide (amyloidogenic pathway) or peptide p3 (non-amyloidogenic pathway). Recent results indicate that phosphorylation of residue T668 in the Thr-Pro motif (“pT668P”) of APP regulates APP processing and Aβ secretion.

FIG. 2 is a schematic diagram of the structure of Pin1. The catalytic (“PPIase”) and binding (“WW”) domains have separate substrate interaction surfaces, shown occupied by space-filled atoms.

FIG. 3 is a schematic diagram of the structure of AICD, determined by NMR, before and after phosphorylation (“Pᵢ”) of residue T668. The isomerized pThr-Pro peptide bond is denoted by “→”.

FIGS. 4A-G are western blots relating to Pin1 binding to the phosphorylated Thr668-Pro motif in APP in vitro and in vivo. FIGS. 4A-C relate to N18 neuroblastoma cells (“APP,” “APP^{T668A}”) transfected, respectively, with an HA-APP or HA-APP^{T668A} construct, arrested at M or the G1/S boundary or left asynchronized (“Asyn”), followed by GST pulldown and immunoblot for APP and APP phosphorylated at T668 (“pT668-APP”) (FIGS. 4A-B), or followed by anti-HA monoclonal antibody immunoprecipitation and then immunoblot for Pin1, APP, and pT668-APP (FIG. 4C). As shown in FIG. 4D, ³⁵S-APP was phosphorylated by Xenopus mitotic extracts (“M”), or mitotic extracts and dephosphorylated with CIP (“M+CIP”), followed by GST pulldown. As shown in FIG. 4E, AICD and AICD^{T668A} phosphorylated by cyclin B/Cdc2 were subjected to GST pulldown directly or after dephosphorylation with CIP, followed by immunoblot with anti-pT668-APP antibodies. FIG. 4F shows that Pin1 binds to the WW domain of AICD. ³²P-AICD was subjected for pulldown with GST-labeled WW domain or GST-labeled PPIase domain. As shown in FIG. 4G, ³²P-AICD was subjected to GST-WW pulldown in the presence of increasing amounts of a pThr668-containing APP phosphopeptide.

FIGS. 5A-D relate to Pin1 catalysis of the isomerization of the pThr668-Pro motif in APP as visualized by NMR spectroscopy. FIG. 5A is a selected region of the E670-H^{N} 2D ROESY spectra of a pThr668-Pro-containing APP peptide at 3 mM in the presence or absence of 0.05 mM GST-Pin1 or its K63A mutant at tₘ=50 or 100 ms. Negative (positive) intensity is denoted in light grey (black). FIG. 5B is an enzyme kinetics scheme for Pin1-catalyzed isomerization of the pThr668-Pro bond. FIG. 5C is a plot relating to the analysis of the kinetic constraints of Pin1-catalyzed cis/trans isomerization of the pThr668-Pro peptide bond. Intensity ratios for the cis (open circles, left axis) and trans (closed circles, right axis) ROESY peaks for E670-H^{N} were plotted verses mixing time, with best-fit curves superimposed. The ratio of cross/diagonal peak intensities for each conformation was calculated based on the equations described in Example 3. FIG. 5D is a schematic diagram of Pin1-catalyzed isomerization between cis and trans conformations of the pThr668-Pro peptide bond (medium gray arrows). Structural models display helix-capping box structures with associated hydrogen bonds. The size and direction of black arrows represent the catalysis reaction accelerated by Pin1.

FIGS. 6A-B are a series of double immunofluorescence stainings showing the colocalization of endogenous APP and Pin1 in CHO-APP cells (FIG. 6A) and H4 neuroglioma cells (FIG. 6B). Cells were doubly stained for Pin1 and APP (top), followed by confocal microscopy (bottom).

FIGS. 7A-B are a series of double immunofluorescence stainings in CHO-APP cells showing the colocalization of APP and clathrin (for clathrin-coated vesicles) (FIG. 7A), or Pin1 and clathrin (FIG. 7B).

FIG. 8A-B are a series of double immunofluorescence stainings in H4 neuroglioma cells showing the colocalization of APP and clathrin (FIG. 8A), or Pin1 and clathrin (FIG. 8B).

FIG. 9A-B are a series of double immunofluorescence stainings in H4 neuroglioma cells showing that EEA1 (for early endosomes) (FIG. 9A) and AP1 (for vesicles recycled from endosomes to trans Golgi networks) (FIG. 9B) colocalize with APP but not with Pin1.

FIGS. 10A-E relate to Pin1 regulation of APP in CHO-APP cells (FIGS. 10A-B), CHO cells (FIG. 10C), and breast cancer cells (FIGS. 10D-F). FIGS. 10A-B are a western blot (FIG. 10A) and graph (FIG. 10B) relating to Pin1 regulation of APP in CHO-APP cells. Cells transfected with a Pin1 construct or vector were left asynchronized or arrested at M, followed by immunoblot for Pin1, APP, and pT668-APP (FIG. 10A), or ELISA measuring total Aβ secretion (see FIG. 10B). FIG. 10C is a graph showing that the overexpression of Pin1 in CHO cells reduces Aβ secretion, which is enhanced by increasing Thr668 phosphorylation. CHO cells were co-transfected with APP and Pin1 or a control vector, and left asynchronized (“Asyn”) or arrested at mitosis (“M”) to increase Thr668 phosphorylation of APP, and the levels of total Aβ secreted into the culture media were measured using ELISA. FIGS. 10D-E are a western blot (FIG. 10D) and a graph (FIG. 10E) relating to Pin1 regulation of APP in breast cancer cells. After Pin1+/+ and Pin1−/− breast cancer cells were transfected with an APP construct, secreted αAPPs was assayed by immunoprecipitation and then immunoblot (FIG. 10D), followed by semi-quantification of αAPPs (FIG. 10E, left) and total Aβ (FIG. 10E, right) by ELISA. FIG. 10F is a western blot relating to the effects of Pin1 knockout on APP processing and Aβ secretion. Similar endogenous levels of total APP, αCTFs, and βCTFs were found in Pin1+/+ and Pin1−/− mouse breast cancer cells.

FIGS. 11A-H show that Pin1 knockout causes age-dependent and selective accumulation of insoluble Aβ42 at multivesicular bodies of neurons, which is accelerated by APP mutant overexpression. FIGS. 11A-H are graphs of insoluble (FIGS. 11A-B and 11E-F) and soluble (FIGS. 11C-D and 11G-H) Aβ levels in mice. Brain tissues were collected from different ages of Pin1−/− and Pin1+/+ littermates (FIGS. 11A-D), or Pin1−/− and Pin1+/+ littermates in the presence of transgenic overexpression of a single copy of APP^{KM670/671NL} (“APP-Tg2576”) (FIGS. 11E-H). Aβ peptides were extracted from brain tissues and Aβ40 (FIGS. 11B and 11F (insoluble) and FIGS. 11D and 11H (soluble)) and Aβ42 (FIGS. 11A and 11E (insoluble) and FIGS. 11C and 11G (soluble)) levels were measured by ELISA and represented as means ±SD.

FIGS. 12A-B are electron micrographs of cortical tissue taken from mice. APP-Tg2576 littermates in the presence of Pin1+/+ (FIG. 12A) or Pin1−/− (FIG. 12B) background were perfused at 7 months old and processed for immunogold-EM using anti-human Aβ42 antibodies. Immunogold particles of Aβ42 are primarily localized to multivesicular bodies (arrows) in dorsal medial cortical neurons. Bar, 500 nm.

FIGS. 13A-H relate to the effect of Pin1 knockout in mice. FIGS. 13A-F show that Pin1 knockout affects APP processing in mice in an age-dependent manner. FIGS. 13A-C are western blots. Brain lysates (FIG. 13A) from 2 month-old (“2 m”) and 6 month-old (“6 m”) APP-Tg2576 mice in the presence of Pin1+/+ or Pin1−/− background and non-transgenic controls were subfractionated into a membrane fraction (FIG. 13B) and a soluble fraction (FIG. 13C), followed by immunoblot with various specific antibodies to detect: mature APP (“APPmat”), immature APP (“APPimm”), βCTF or αCTF phosphorylated or unphosphorylated at residue T668 (“βCTF,” “αCTF” (unphosphorylated); “pT668-βCTF,” “pT668-αCTF” (phosphorylated)), total APPs, αAPPs, βAPPs, Pin1, or tubulin. FIGS. 13D-E are graphs of total APPs (FIG. 13D), αAPPs (FIG. 13E), and βAPPs (FIG. 13F) in 2- and 6-month-old mice. Amounts were semi-quantified with NIH Image and presented with Pin1+/+ controls as 100% (white bars). FIGS. 13G-H are schematic diagrams modeling APP processing in the presence (FIG. 13G) and absence (FIG. 13H) of proper Pin1 function. Although the pTh668-Pro motif of APP tends to be in cis after phosphorylation, functional Pin1 would greatly accelerate cis/trans isomerization, which might favor non-amyloidogenic APP processing (FIG. 13G). Without proper Pin1 function, the cis pThr668-Pro motif would not be isomerized to trans in a timely manner, which might favor amyloidogenic APP processing (FIG. 13H).

FIGS. 14A-C are mass spectrometry spectra. LC-EMS shows 99% cyclization of synthetic VpTPEER (SEQ ID NO: 1) peptide. FIG. 14A is a single-stage electrospray mass spectrometry spectrum showing peak at the theoretical molecular weight of the cyclic peptide (791.8 amu) along with multiple impurities from the reaction mixture. FIG. 14B shows the selective detection of phosphate-containing species, showing the cyclic peptide as by far the dominant component (linear form at 809 is less than 3%). FIG. 14C shows the retention time (15.31 min) of the cyclic peptide on a capillary C18 reverse phase column, 0.1% formic acid, 5-35% acetonitrile gradient.

FIGS. 15A-B show that phosphopeptide pThr-Pro mimetics have enriched cis content. FIG. 16A is a series of TOCSY (left) and 31P (right) spectra of cyclic (left) and linear (right) pAICD phosphopeptides. FIG. 16B is a region of a ROESY spectrum of Pin1-Inh01 showing cis-distinguishing cross peaks.

FIGS. 16A-C are spectra showing that the cyclic form of T668-phosphorylated AICD peptide binds to the WW domain of Pin1 in a manner consistent with intrinsic cis/trans populations of 30%/70%. Regions of overlaid ¹⁵N—¹H HSQC spectra for the selected residues (residues Y23 (FIG. 16A), R14 (FIG. 16B), and W34 (FIG. 16C)) are representative of the consistent trends in peak shifts upon saturation with the linear T668-phosphorylated peptide (“pAICD”) compared with saturation with the cyclic form of the peptide (“cyclic pAICD”) relative to free WW domain (“apo”).

### DETAILED DESCRIPTION OF THE INVENTION

One aspect of the present invention relates to a method of inhibiting amyloidogenic processing of amyloid precursor protein. This method involves accelerating cis/trans isomerization of the amyloid precursor protein at a phosphorylated serine/threonine-proline motif under conditions effective to inhibit amyloidogenic processing of the amyloid precursor protein.

Regulation of APP Structure and Function by Phosphorylation, Especially on the Thr668-Pro Motif.

Increasing evidence suggests that APP processing and function is modulated by phosphorylation within the APP intracellular domain (“AICD”), including phosphorylation on the Thr668-Pro motif (Pastorino & Lu, “Phosphorylation of the Amyloid Precursor Protein (APP): Is This a Mechanism in Favor or Against Alzheimer's Disease,” Neurosci Res Commun 35:213-231 (2005); Suzuki et al., “Cell Cycle-dependent Regulation of the Phosphorylation and Metabolism of the Alzheimer Amyloid Precursor Protein,” Embo J 13(5):1114-22 (1994); Phiel et al., “GSK-3a Regulates Production of Alzheimer's Disease Amyloid-β Peptides,” Nature 423(6938):435-9 (2003); Aplin et al., “In Vitro Phosphorylation of the Cytoplasmic Domain of the Amyloid Precursor Protein by Glycogen Synthase Kinase-3β,” J Neurochem 67(2):699-707 (1996); Iijima et al., “Neuron-specific Phosphorylation of Alzheimer's β-Amyloid Precursor Protein by Cyclin-dependent Kinase 5,” J Neurochem 75(3):1085-91 (2000); Standen et al., “Phosphorylation of Thr(668) in the Cytoplasmic Domain of the Alzheimer's Disease Amyloid Precursor Protein by Stress-activated Protein Kinase 1b (Jun N-terminal Kinase-3),” J Neurochem 76(1):316-20 (2001); Lee et al., “APP Processing Is Regulated by Cytoplasmic Phosphorylation,” J Cell Biol 163(1):83-95 (2003); Kimberly et al., “Physiological Regulation of the β-Amyloid Precursor Protein Signaling Domain by c-Jun N-terminal Kinase JNK3 During Neuronal Differentiation,” J Neurosci 25(23):5533-43 (2005), which are hereby incorporated by reference in their entirety). Functionally, Thr668 phosphorylation of APP has been implicated in neural function and/or AD pathogenesis (Kimberly et al., “Physiological Regulation of the β-Amyloid Precursor Protein Signaling Domain by c-Jun N-terminal Kinase JNK3 During Neuronal Differentiation,” J Neurosci 25(23):5533-43 (2005); Ando et al., “Role of Phosphorylation of Alzheimer's Amyloid Precursor Protein During Neuronal Differentiation,” J Neurosci 19(11):4421-7 (1999); Lee et al., “APP Processing Is Regulated by Cytoplasmic Phosphorylation,” J Cell Biol 163(1):83-95 (2003); Lee & Tsai, “Cdk5: One of the Links Between Senile Plaques and Neurofibrillary Tangles?,” J Alzheimers Dis 5(2):127-37 (2003); Inomata et al., “A Scaffold Protein JIP-1b Enhances Amyloid Precursor Protein Phosphorylation by JNK and Its Association with Kinesin Light Chain 1,” J Biol Chem 278(25):22946-55 (2003); Vincent et al., “Mitotic Mechanisms in Alzheimer's Disease?,” J Cell Biol 132(3):413-25 (1996); Lee et al., “Neurotoxicity Induces Cleavage of p35 to p25 by Calpain,” Nature 405(6784):360-4 (2000); Davis, “Signal Transduction by the JNK Group of MAP Kinases,” Cell 103(2):239-52 (2000); Zhu et al., “Activation and Redistribution of c-Jun N-terminal Kinase/Stress Activated Protein Kinase in Degenerating Neurons in Alzheimer's Disease,” J Neurochem 76(2):435-41 (2001); Okazawa & Estus, “The JNK/c-Jun Cascade and Alzheimer's Disease,” Am J Alzheimers Dis Other Demen 17(2):79-88 (2002); Zhu et al., “The Role of Mitogen-activated Protein Kinase Pathways in Alzheimer's Disease,” Neurosignals 11(5):270-81 (2002); Lu et al., “Proline-directed Phosphorylation and Isomerization in Mitotic Regulation and in Alzheimer's Disease,” BioEssays 25:174-181 (2003), which are hereby incorporated by reference in their entirety). For example, pThr668-APP and BACE1 colocalize in enlarged endosomes in AD and cultured primary neurons. However, the significance and regulation of Thr668-Pro phosphorylation during APP processing and AD pathogenesis was unknown.

A Unique Conformational Switch of the pThr668-Pro Motif in APP.

Although Pro-directed phosphorylation has long been proposed to regulate protein function via inducing conformational changes, little was known about the nature of the conformational changes and whether they were further regulated until recently (Lu, “Pinning Down Cell Signaling, Cancer and Alzheimer's Disease,” Trends Biochem Sci 29:200-209 (2004); Wulf et al., “Phosphorylation-specific Prolyl Isomerization: Is There an Underlying Theme?,” Nature Cell Biol 7:435-41 (2005); Lu et al., “Pinning Down the Proline-directed Phosphorylation Signaling,” Trends Cell Biol 12:164-172 (2002), which are hereby incorporated by reference in their entirety). Proline residues in folded proteins can exist in two distinct isomers, cis and trans, and therefore can provide a potential backbone switch that is controlled by cis/trans isomerization (Lu et al., “Pinning Down the Proline-directed Phosphorylation Signaling,” Trends Cell Biol 12:164-172 (2002), which is hereby incorporated by reference in its entirety). This intrinsically rather slow conversion can be catalyzed by cis/trans peptidyl-prolyl isomerases (“PPIases”) (Fischer, “Chemical Aspects of Peptide Bond Isomerisation,” Chem Soc Rev 29:119-27 (2000); Hunter, “Prolyl Isomerases and Nuclear Function,” Cell 92(2): 141-3 (1998); Lu et al., “Pinning Down Proline-directed Phosphorylation Signaling,” Trends Cell Biol 12(4):164-72 (2002), which are hereby incorporated by reference in their entirety). Isomerization of Ser/Thr-Pro motifs is especially important because Pro-directed kinases and phosphatases are conformation specific, only acting on trans Ser/Thr-Pro motifs (Zhou et al., “Pin1-dependent Prolyl Isomerization Regulates Dephosphorylation of Cdc25C and Tau Proteins,” Mol Cell 6:873-883 (2000); Weiwad et al., “Evidence that the Substrate Backbone Conformation Is Critical to Phosphorylation by p42 MAP Kinase,” FEBS Lett 478(1-2):39-42 (2000); Brown et al., “The Structural Basis for Specificity of Substrate and Recruitment Peptides for Cyclin-dependent Kinases,” Nat Cell Biol 1(7):438-43 (1999), which are hereby incorporated by reference in their entirety). Importantly, phosphorylation further slows down the already slow isomerization reaction of Ser/Thr-Pro bonds (Yaffe et al., “Sequence-specific and Phosphorylation-dependent Proline Isomerization: A Potential Mitotic Regulatory Mechanism,” Science 278:1957-1960 (1997); Schutkowski et al., “Role of Phosphorylation in Determining the Backbone Dynamics of the Serine/Threonine-Proline Motif and Pin1 Substrate Recognition,” Biochemistry 37(16):5566-75 (1998), which are hereby incorporated by reference in their entirety), and also renders the phosphopeptide bond resistant to the catalytic action of cyclophilin, FKBP, or parvulin (Yaffe et al., “Sequence-specific and Phosphorylation-dependent Proline Isomerization: A Potential Mitotic Regulatory Mechanism,” Science 278:1957-1960 (1997); Uchida et al., “Identification and Characterization of a 14 kDa Human Protein as a Novel Parvulin-like Peptidyl Prolyl Cis/Trans Isomerase,” FEBS Lett 446:278-82 (1999), which are hereby incorporated by reference in their entirety). Hence, there is a need for phosphorylation-specific PPIases (Lu et al., “Pinning Down the Proline-directed Phosphorylation Signaling,” Trends Cell Biol 12:164-172 (2002), which is hereby incorporated by reference in its entirety).

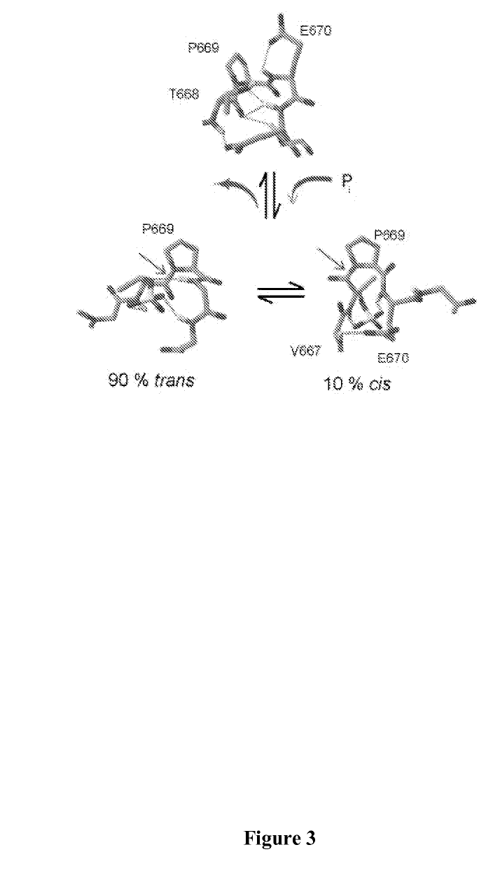

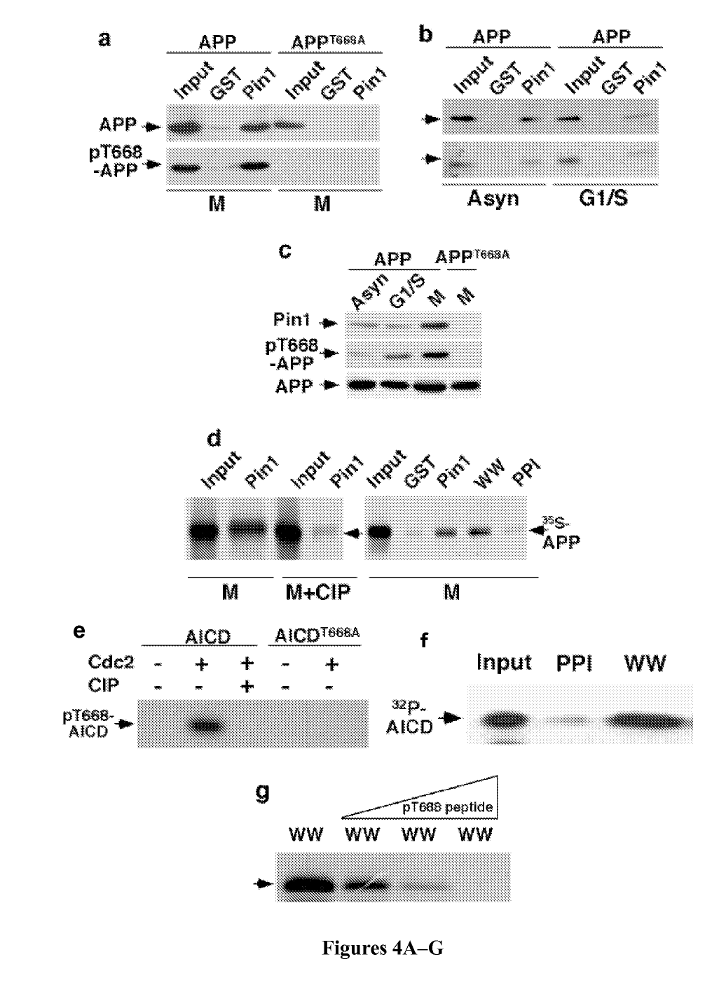

As shown in FIG. 3, the Thr668-Pro motif in APP exists in the trans conformation in a stable helix-capping box structure (Ramelot et al., “Transient Structure of the Amyloid Precursor Protein Cytoplasmic Tail Indicates Preordering of Structure for Binding to Cytosolic Factors,” Biochemistry 39(10):2714-25 (2000), which is hereby incorporated by reference in its entirety). Phosphorylation of APP at T668 causes the pThr668-Pro peptide bond to partition into two populations, 10% cis and 90% trans, and the cis and trans conformations are in slow exchange (Ramelot & Nicholson, “Phosphorylation-induced Structural Changes in the Amyloid Precursor Protein Cytoplasmic Tail Detected by NMR,” J Mol Biol 307(3):871-84 (2001), which is hereby incorporated by reference in its entirety). It was hypothesized that this conformational switch involving the pThr668-Pro motif in APP may serve as a novel mechanism to regulate APP processing and Aβ production, possibly mediated by a phosphorylation-specific PPIase. The present invention demonstrates that this is indeed the case (see also Pastorino & Lu, “Phosphorylation of the Amyloid Precursor Protein (APP): Is This a Mechanism in Favor or Against Alzheimer's Disease,” Neurosci Res Commun 35:213-231 (2005), which is hereby incorporated by reference in its entirety).

Pin1 is Pivotal in Protecting Against Age-Dependent Tauopathy and Neurodegeneration.

The abundant Pro-directed phosphorylation in AD and its strong connection with aberrant mitotic events led to the hypothesis that Pin1 plays a role in the pathogenesis of AD (Lu et al., “The Prolyl Isomerase Pin1 Restores the Function of Alzheimer-associated Phosphorylated Tau Protein,” Nature 399:784-788 (1999), which is hereby incorporated by reference in its entirety). Indeed, in normal brains, Pin1 is mainly expressed in most neurons at unusually high levels and is in the soluble fraction (Lu et al., “The Prolyl Isomerase Pin1 Restores the Function of Alzheimer-associated Phosphorylated Tau Protein,” Nature 399:784-788 (1999); Lu et al., “A Human Peptidyl-prolyl Isomerase Essential for Regulation of Mitosis,” Nature 380(6574):544-7 (1996); Wulf et al., “Pin1 Is Overexpressed in Breast Cancer and Potentiates the Transcriptional Activity of Phosphorylated c-Jun Towards the Cyclin D1 Gene,” EMBO J 20:3459-3472 (2001); Ryo et al., “Pin1 Regulates Turnover and Subcellular Localization of O-Catenin by Inhibiting Its Interaction with APC,” Nature Cell Biol 3:793-801 (2001); Thorpe et al., “Shortfalls in the Peptidyl-prolyl Cis-trans Isomerase Protein Pin1 in Neurons are Associated with Frontotemporal Dementias,” Neurobiol Dis 17(2):237-49 (2004), which are hereby incorporated by reference in their entirety). However, in AD brains and related disorders, cytoplasmic Pin1 is increased and co-localizes and co-purifies with neurofibrillary tangles, resulting in depletion of soluble Pin1 (Lu et al., “The Prolyl Isomerase Pin1 Restores the Function of Alzheimer-associated Phosphorylated Tau Protein,” Nature 399:784-788 (1999); Thorpe et al., “Shortfalls in the Peptidyl-prolyl Cis-trans Isomerase Protein Pin1 in Neurons are Associated with Frontotemporal Dementias,” Neurobiol Dis 17(2):237-49 (2004); Thorpe et al., “Utilizing the Peptidyl-prolyl Cis-trans Isomerase Pin1 as a Probe of Its Phosphorylated Target Proteins. Examples of Binding to Nuclear Proteins in a Human Kidney Cell Line and to Tau in Alzheimer's Diseased Brain,” J Histochem Cytochem 49(1):97-108 (2001); Ramakrishnan et al., “Pin1 Colocalization with Phosphorylated Tau in Alzheimer's Disease and Other Tauopathies,” Neurobiol Dis 14(2):251-64 (2003), which are hereby incorporated by reference in their entirety). The significance of this Pin1 depletion in AD is further underscored by the findings that Pin1 regulates the biological function and dephosphorylation of some MPM-2 antigens, including tau, Cdc25C, and the C-terminal domain of RNA Pol II (Zhou et al., “Pin1-dependent Prolyl Isomerization Regulates Dephosphorylation of Cdc25C and Tau Proteins,” Mol Cell 6:873-883 (2000); Lu et al., “The Prolyl Isomerase Pin1 Restores the Function of Alzheimer-associated Phosphorylated Tau Protein,” Nature 399:784-788 (1999); Xu et al., “Pin1 Modulates the Structure and Function of Human RNA Polymerase II,” Genes Dev 17:2765-2776 (2003); Kops et al., “Pin1 Enhances the Dephosphorylation of the C-terminal Domain of the RNA Polymerase II by Fcp1,” FEBS Lett 513:305-311 (2002), which are hereby incorporated by reference in their entirety). Pin1 binds to pThr231-tau and restores its ability to bind microtubules and to promote microtubule assembly in vitro (Lu et al., “The Prolyl Isomerase Pin1 Restores the Function of Alzheimer-associated Phosphorylated Tau Protein,” Nature 399:784-788 (1999), which is hereby incorporated by reference in its entirety). Furthermore, Pin1 also facilitates tau dephosphorylation by PP2A because PP2A can only dephosphorylate trans pSer/Thr-Pro motifs (Zhou et al., “Pin1-dependent Prolyl Isomerization Regulates Dephosphorylation of Cdc25C and Tau Proteins,” Mol Cell 6:873-883 (2000); Sontag et al., “Molecular Interactions Among Protein Phosphatase 2A, Tau, and Microtubules. Implications for the Regulation of Tau Phosphorylation and the Development of Tauopathies,” J Biol Chem 274(36):25490-8 (1999), which are hereby incorporated by reference in their entirety). In addition, Pin1 is important for maintaining the stability of β-catenin (Ryo et al., “Pin1 Regulates Turnover and Subcellular Localization of β-Catenin by Inhibiting Its Interaction with APC,” Nature Cell Biol 3:793-801 (2001), which is hereby incorporated by reference in its entirety), a protein that is destabilized by presenilin-1 mutations and long implicated in AD (Zhang et al., “Destabilization of β-Catenin by Mutations in Presenilin-1 Potentiates Neuronal Apoptosis,” Nature 395(6703):698-702 (1998); De Ferrari & Inestrosa, “Wnt Signaling Function in Alzheimer's Disease,” Brain Res Brain Res Rev 33(1): 1-12 (2000), which are hereby incorporated by reference in their entirety). These results suggest that Pin1 may have neuroprotective functions against neurodegeneration (Lu et al., “Proline-directed Phosphorylation and Isomerization in Mitotic Regulation and in Alzheimer's Disease,” BioEssays 25:174-181 (2003), which is hereby incorporated by reference in its entirety).

This is also evidenced by Pin1's distribution in human brains and the neuronal phenotypes of Pin1-deficient (“Pin1−/−”) mice. Neurons in different subregions of the hippocampus are known to have differential vulnerability to neurofibrillary degeneration in AD (Pearson et al., “Anatomical Correlates of the Distribution of the Pathological Changes in the Neocortex in Alzheimer Disease,” Proc Nat'l Acad Sci USA 82(13):4531-4 (1985); Hof & Morrison, “Neocortical Neuronal Subpopulations Labeled by a Monoclonal Antibody to Calbindin Exhibit Differential Vulnerability in Alzheimer's Disease,” Exp Neurol 111(3):293-301 (1991); Arriagada et al., “Distribution of Alzheimer-type Pathologic Changes in Nondemented Elderly Individuals Matches the Pattern in Alzheimer's Disease,” Neurology 42(9):1681-8 (1992); Davies et al., “The Effect of Age and Alzheimer's Disease on Pyramidal Neuron Density in the Individual Fields of the Hippocampal Formation,” Acta Neuropathol (Berl) 83(5):510-7 (1992); Thal et al., “Sequence of Aβ-protein Deposition in the Human Medial Temporal Lobe,” J Neuropathol Exp Neurol 59(8):733-48 (2000), which are hereby incorporated by reference in their entirety). Pin1 expression inversely correlates with the predicted neuronal vulnerability in normally aged brains and also with actual neurofibrillary degeneration in AD (Liou et al., “Role of the Prolyl Isomerase Pin1 in Protecting Against Age-dependent Neurodegeneration,” Nature 424:556-561 (2003), which is hereby incorporated by reference in its entirety). Moreover, Pin1−/− mice develop progressive age-dependent neuropathy characterized by motor and behavioral deficits, tau hyperphosphorylation, tau filament formation, and neuronal degeneration (Liou et al., “Role of the Prolyl Isomerase Pin1 in Protecting Against Age-dependent Neurodegeneration,” Nature 424:556-561 (2003), which is hereby incorporated by reference in its entirety). These phenotypes resemble those in many tau-related transgenic mice (Ishihara et al., “Age-dependent Emergence and Progression of a Tauopathy in Transgenic Mice Overexpressing the Shortest Human Tau Isoform,” Neuron 24(3):751-62 (1999); Lewis et al., “Neurofibrillary Tangles, Amyotrophy and Progressive Motor Disturbance in Mice Expressing Mutant (P3011) Tau Protein,” Nat Genet 25(4):402-5 (2000); Lewis et al., “Enhanced Neurofibrillary Degeneration in Transgenic Mice Expressing Mutant Tau and APP,” Science 293(5534):1487-91 (2001); Gotz et al., “Formation of Neurofibrillary Tangles in P3011 Tau Transgenic Mice Induced by Aβ 42 Fibrils,” Science 293(5534):1491-5 (2001); Cruz et al., “Aberrant Cdk5 Activation by p25 Triggers Pathological Events Leading to Neurodegeneration and Neurofibrillary Tangles,” Neuron 40:471-483 (2003); Geschwind, “Tau Phosphorylation, Tangles, and Neurodegeneration: The Chicken or the Egg,” Neuron 40:457-460 (2003), which are hereby incorporated by reference in their entirety). Thus, Pin1 is pivotal for protecting against age-dependent neurodegeneration and the tau-related pathology.

Pin1-Catalyzed Prolyl Isomerization Regulates APP Processing and Aβ Production.

The finding that Pin1 is important for protecting against tauopathy and neurodegeneration (Lu, “Pinning Down Cell Signaling, Cancer and Alzheimer's Disease,” Trends Biochem Sci 29:200-209 (2004); Lu et al., “The Prolyl Isomerase Pin1 Restores the Function of Alzheimer-associated Phosphorylated Tau Protein,” Nature 399:784-788 (1999); Liou et al., “Role of the Prolyl Isomerase Pin1 in Protecting Against Age-dependent Neurodegeneration,” Nature 424:556-561 (2003), which are hereby incorporated by reference in their entirety) and that Thr668 phosphorylation is increased during mitosis (Suzuki et al., “Cell Cycle-dependent Regulation of the Phosphorylation and Metabolism of the Alzheimer Amyloid Precursor Protein,” Embo J 13(5): 1114-22 (1994), which is hereby incorporated by reference in its entirety) suggest that Pin1 might act on the pThr668-Pro motif to regulate APP processing and Aβ production. In contrast to previous data (Akiyama et al., “Pin1 Promotes Production of Alzheimer's Amyloid β From β-Cleaved Amyloid Precursor Protein,” Biochem Biophys Res Commun 336(2):521-9 (2005), which is hereby incorporated by reference in its entirety), the present invention demonstrates that Pin1 binds to the pThr668-Pro motif in APP in vitro and in vivo (Examples 7-8; see also Pastorino et al., “The Prolyl Isomerase Pin1 Regulates Amyloid Precursor Protein Processing and Amyloid-O Production,” Nature 440(7083):528-34 (2006), which is hereby incorporated by reference in its entirety). Moreover, NMR spectroscopy directly visualizes Pin1-catalyzed pThr668-Pro isomerization (Example 9; see also Pastorino et al., “The Prolyl Isomerase Pin1 Regulates Amyloid Precursor Protein Processing and Amyloid-β Production,” Nature 440(7083):528-34 (2006), which is hereby incorporated by reference in its entirety). Pin1 accelerates the cis/trans isomerization rate by several orders of magnitude over the typical uncatalyzed isomerization rates for pThr-Pro peptides (Schutkowski et al., “Role of Phosphorylation in Determining the Backbone Dynamics of the Serine/Threonine-Proline Motif and Pin1 Substrate Recognition,” Biochemistry 37(16):5566-75 (1998), which is hereby incorporated by reference in its entirety) and dramatically reduces the average lifetime of the cis (˜0.05 s) and trans (˜0.5 s) isomeric states. The k_{ct}^{cat} and k_{tc}^{cat} rates differ by 10-fold, as expected based on the equilibrium populations of free cis and trans. The present invention provides the first direct atomic level demonstration of Pin1-catalyzed conformational regulation of its substrates, particularly pT668-Pro (Pastorino et al., “The Prolyl Isomerase Pin1 Regulates Amyloid Precursor Protein Processing and Amyloid-β Production,” Nature 440(7083):528-34 (2006), which is hereby incorporated by reference in its entirety).

Subcellular sublocalization studies reveal that Pin1 and APP co-localize prominently at the plasma membrane and clathrin-coated vesicles, but not at endosomes or subsequent structures (Example 10; see also Pastorino et al., “The Prolyl Isomerase Pin1 Regulates Amyloid Precursor Protein Processing and Amyloid-β Production,” Nature 440(7083):528-34 (2006), which is hereby incorporated by reference in its entirety). Since APP is processed by non-amyloidogenic α-secretases mainly at the plasma membrane and by amyloidogenic β- and γ-secretases at endosomes and other structures (Hardy & Selkoe, “The Amyloid Hypothesis of Alzheimer's Disease: Progress and Problems on the Road to Therapeutics,” Science 297(5580):353-6 (2002); Mattson, “Pathways Towards and Away from Alzheimer's Disease,” Nature 430(7000):631-9 (2004), which are hereby incorporated by reference in their entirety), these results suggest that Pin1 may regulate APP processing and Aβ production. Indeed, Pin1 overexpression in CHO-APP and CHO cells significantly reduces Aβ secretion, especially from mitotic cells where Thr668 phosphorylation is elevated. In contrast, Pin1 knockout in cells decreases αAPPs, but increases Aβ secretion. Moreover, knockout of Pin1 alone or in combination with overexpression of mutant APP in mice increases amyloidogenic APP processing and selectively elevates insoluble Aβ42 (a major toxic species) in brains in an age-dependent manner, with Aβ42 being prominently localized to multivesicular bodies of neurons (Example 11; see also Pastorino et al., “The Prolyl Isomerase Pin1 Regulates Amyloid Precursor Protein Processing and Amyloid-P Production,” Nature 440(7083):528-34 (2006), which is hereby incorporated by reference in its entirety), where Aβ42 is known to be in human AD brains and the brains of APP-Tg2576 mice (a transgenic mouse strain overexpressing a mutant APP) before β-amyloid plaque pathology (Takahashi et al., “Intraneuronal Alzheimer Aβ42 Accumulates in Multivesicular Bodies and Is Associated with Synaptic Pathology,” Am J Pathol 161(5):1869-79 (2002), which is hereby incorporated by reference in its entirety).

The present invention demonstrates that Pin1-catalyzed prolyl isomerization is a novel mechanism to regulate APP processing and Aβ production. Given that Pin1 is downregulated and/or inhibited by oxidation in AD neurons (Lu et al., “The Prolyl Isomerase Pin1 Restores the Function of Alzheimer-associated Phosphorylated Tau Protein,” Nature 399:784-788 (1999); Sultana et al., “Oxidative Modification and Down-regulation of Pin1 in Alzheimer's Disease Hippocampus: A Redox Proteomics Analysis,” Neurobiol Aging 27(7):918-25 (2006 (Epub 2005)), which are hereby incorporated by reference in their entirety), that Pin1 knockout causes age-dependent tauopathy phenotype and neurodegeneration (Zhou et al., “Pin1-dependent Prolyl Isomerization Regulates Dephosphorylation of Cdc25C and Tau Proteins,” Mol Cell 6:873-883 (2000); Liou et al., “Role of the Prolyl Isomerase Pin1 in Protecting Against Age-dependent Neurodegeneration,” Nature 424:556-561 (2003), which are hereby incorporated by reference in their entirety), and that Pin1 genetic changes appear to associate with reduced Pin1 levels and increased risk for late-onset AD (Segat et al., “Pin1 Promoter Polymorphisms are Associated with Alzheimer's Disease,” Neurobiol Aging 28(1):69-74 (2007 (Epub 2005)); Wijsman et al., “Evidence for a Novel Late-onset Alzheimer Disease Locus on Chromosome 19p13.2,” Am J Hum Genet 75(3):398-409 (2004), which are hereby incorporated by reference in their entirety), these results indicate that Pin1 aberrations may link both tangle and plaque pathologies. In distinction from many other AD mouse models where transgenic overexpression of specific proteins elicits AD related phenotypes (Wong et al., “Genetically Engineered Mouse Models of Neurodegenerative Diseases,” Nat Neurosci 5(7):633-9 (2002); Duff et al., “Increased Amyloid-β42(43) in Brains of Mice Expressing Mutant Presenilin 1,” Nature 383(6602):710-3 (1996); Games et al., “Alzheimer-type Neuropathology in Transgenic Mice Overexpressing V717F β-Amyloid Precursor Protein,” Nature 373(6514):523-7 (1995); Hsiao et al., “Correlative Memory Deficits, Aβ Elevation, and Amyloid Plaques in Transgenic Mice,” Science 274(5284):99-102 (1996); Borchelt et al., “Familial Alzheimer's Disease-linked Presenilin 1 Variants Elevate Aβ1-42/1-40 Ratio in Vitro and in Vivo,” Neuron 17(5):1005-13 (1996); Chen et al., “A Learning Deficit Related to Age and β-Amyloid Plaques in a Mouse Model of Alzheimer's Disease,” Nature 408(6815):975-9 (2000); Ishihara et al., “Age-dependent Emergence and Progression of a Tauopathy in Transgenic Mice Overexpressing the Shortest Human Tau Isoform,” Neuron 24(3):751-62 (1999); Lewis et al., “Neurofibrillary Tangles, Amyotrophy and Progressive Motor Disturbance in Mice Expressing Mutant (P3011) Tau Protein,” Nat Genet 25(4):402-5 (2000); Gotz et al., “Tau Filament Formation in Transgenic Mice Expressing P301L Tau,” J Biol Chem 276(1):529-34 (2001); Tanemura et al., “Neurodegeneration with Tau Accumulation in a Transgenic Mouse Expressing V337M Human Tau,” J Neurosci 22(1): 133-41 (2002); Lewis et al., “Enhanced Neurofibrillary Degeneration in Transgenic Mice Expressing Mutant Tau and APP,” Science 293(5534):1487-91 (2001); Gotz et al., “Formation of Neurofibrillary Tangles in P3011 Tau Transgenic Mice Induced by Aβ 42 Fibrils,” Science 293(5534):1491-5 (2001); Geschwind, “Tau Phosphorylation, Tangles, and Neurodegeneration: The Chicken or the Egg,” Neuron 40:457-460 (2003); Ahlijanian et al., “Hyperphosphorylated Tau and Neurofilament and Cytoskeletal Disruptions in Mice Overexpressing Human p25, an Activator of Cdk5,” Proc Nat'l Acad Sci USA, 97(6):2910-5 (2000); Lucas et al., “Decreased Nuclear O-Catenin, Tau Hyperphosphorylation and Neurodegeneration in GSK-3β Conditional Transgenic Mice,” Embo J 20(1-2):27-39 (2001); Lim et al., “FTDP-17 Mutations in Tau Transgenic Mice Provoke Lysosomal Abnormalities and Tau Filaments in Forebrain,” Mol Cell Neurosci 18(6):702-14 (2001); Allen et al., “Abundant Tau Filaments and Nonapoptotic Neurodegeneration in Transgenic Mice Expressing Human P301S Tau Protein,” J Neurosci 22(21):9340-51 (2002), which are hereby incorporated by reference in their entirety), Pin1−/− mice are the first gene knockout mouse model that displays both tau and Aβ-related phenotypes resembling those in AD (Liou et al., “Role of the Prolyl Isomerase Pin1 in Protecting Against Age-dependent Neurodegeneration,” Nature 424:556-561 (2003), which is hereby incorporated by reference in its entirety). These findings provide significant new insight into the pathogenesis and treatment of Alzheimer's disease.

One aspect of the present invention relates to a method of inhibiting amyloidogenic processing of APP by accelerating the cis/trans isomerization of APP at a pSer/Thr-Pro motif.

Acceleration of the cis/trans isomerization of APP at a pSer/Thr-Pro motif according to this and all aspects of the present invention refers to any increase in the isomerization rate.

Acceleration may be carried out by contacting the pSer/Thr-Pro motif with an isomerization catalyst.

Suitable catalysts include, for example, Pin1, Pin1 homologues, catalytic antibodies, and RNA aptamers.

Pin1 includes, for example, GenBank Accession No. U49070 (Lu et al., “A Human Peptidyl-prolyl Isomerase Essential for Regulation of Mitosis,” Nature 380(6574):544-7 (1996), which is hereby incorporated by reference in its entirety). Pin1 as used herein includes Pin1 variants, i.e., Pin1 that has been modified by, for example, the deletion or addition of amino acids that have minimal influence on the catalytic properties, secondary structure and hydropathic nature of Pin1. For example, Pin1 may be conjugated to a signal (or leader) sequence at its N-terminal end that co-translationally or post-translationally directs transfer of Pin1. Pin1 may also be conjugated to a linker or other sequence for ease of synthesis, purification, or identification. Considerable mutagenesis has been carried out on Pin1 (see Zhou et al., “Pin1-dependent Prolyl Isomerization Regulates Dephosphorylation of Cdc25C and Tau Proteins,” Mol Cell 6:873-883 (2000) (esp. Table 1); Lu et al., “A Function of WW Domains as Phosphoserine- or Phosphothreonine-binding Modules,” Science 283:1325-1328 (1999) (esp. Table 2), which are hereby incorporated by reference in their entirety). The PPIase domain (i.e., the catalytic domain) is necessary and sufficient to carry out the essential function of Pin1 (Zhou et al., “Pin1-dependent Prolyl Isomerization Regulates Dephosphorylation of Cdc25C and Tau Proteins,” Mol Cell 6:873-883 (2000), which is hereby incorporated by reference in its entirety). Thus, the present invention contemplates Pin1 variants that, e.g., retain the PPIase but are modified at other locations (including, e.g., variants in which the WW domain has been deleted or rendered inactive). Variants in which the PPIase domain has been modified to increase its catalytic activity are also contemplated. Specific residues that play key roles in Pin1-substrate interactions include, e.g., Arg68 and Arg69; specific residues that play key roles in catalysis include, e.g., H59, K₆₃, C113, L122, M130, F134, H157 and S154 (Zhou et al., “Pin1-dependent Prolyl Isomerization Regulates Dephosphorylation of Cdc25C and Tau Proteins,” Mol Cell 6:873-883 (2000), which is hereby incorporated by reference in its entirety). Point mutations of certain residues conserved between Pin1 and bacterial parvulin (i.e., L60P and L61P), or that are unique to Pin1 (i.e., S67E, or S71P) disrupt the ability of Pin1 to isomerize pThr-Pro motifs or to rescue yeast lethal phenotypes even under overexpression, indicating an essential role of these residues in Pin1 stability and/or PPIase activity (Lu et al., “A Function of WW Domains as Phosphoserine- or Phosphothreonine-binding Modules,” Science 283:1325-8 (1999); Zhou et al., “Pin1-dependent Prolyl Isomerization Regulates Dephosphorylation of Cdc25C and Tau Proteins,” Mol Cell 6:873-83 (2000), which are hereby incorporated by reference in their entirety). In addition, point mutations of certain residues in the Pin1 WW domain (W10R and Y23A) have no effect on the PPIase activity, but disrupt the ability of Pin1 to bind phosphoproteins (Lu et al., “A Function of WW Domains as Phosphoserine- or Phosphothreonine-binding Modules,” Science 283:1325-8 (1999), which is hereby incorporated by reference in its entirety).

As will be apparent to one of ordinary skill in the art, Pin1 homologues are cis/trans isomerization catalysts that, like Pin1, catalyze cis/trans isomerization of pSer/Thr-Pro motifs. Suitable Pin1 homologues according this and all aspects of the present invention include, for example, GenBank Accession No. AB009691 (Pin1 of mouse) (Fujimori et al., “Mice Lacking Pin1 Develop Normally, but are Defective in Entering Cell Cycle from G(0) Arrest,” Biochem Biophys Res Commun 265(3):658-63 (1999), which is hereby incorporated by reference in its entirety); GenBank Accession No. AAC28408 (DODO of Drosophila melanogaster) (Maleszka et al., “The Drosophila melanogaster Dodo (Dod) Gene, Conserved in Humans, is Functionally Interchangeable with the ESS1 Cell Division Gene of Saccharomyces cerevisiae,” Proc Nat'l Acad Sci USA 93(1):447-51 (1996), which is hereby incorporated by reference in its entirety); GenBank Accession No. AJ133755 (Par13 of Digitalis lanata, particularly preferred because this lacks a WW domain) (Metzner et al., “Functional Replacement of the Essential ESS1 in Yeast by the Plant Parvulin DlPar13,” J Biol Chem 276(17):13524-9 (2001), which is hereby incorporated by reference in its entirety); GenBank Accession No. AAD20122 (Pin1At of Arabidopsis thaliana) (Landrieu et al., “The Arabidopsis thaliana PIN1At Gene Encodes a Single-domain Phosphorylation-dependent Peptidyl Prolyl Cis/Trans Isomerase,” J Biol Chem 275(14):10577-81 (2000), which is hereby incorporated by reference in its entirety); GenBank Accession Nos. S52764 and CAA59961 (Ess1/Ptf1 of Saccharomyces cerevisiae) (Hanes et al., “Sequence and Mutational Analysis of ESS1, a Gene Essential for Growth in Saccharomyces cerevisiae,” Yeast 5(1):55-72 (1989); Hani et al., “PTF1 Encodes an Essential Protein in Saccharomyces cerevisiae, Which Shows Strong Homology with a New Putative Family of PPIases,” FEBS Lett 365(2-3):198-202 (1995), which are hereby incorporated by reference in their entirety); and GenBank Accession No. CAA20742 (Ssp1 of Neurospora crassa) (Kops et al., “Ssp1, a Site-specific Parvulin Homolog from Neurospora crassa Active in Protein Folding,” J Biol Chem 273(48):31971-6) (1998), which is hereby incorporated by reference in its entirety).

Suitable isomerization catalysts may also be designed and prepared by one of ordinary skill in the art, based on the transition state analog of the pSer/Thr-Pro motif.

Suitable isomerization catalysts include, e.g., catalytic antibodies (Benkovic et al., “The Enzymic Nature of Antibody Catalysis: Development of Multistep Kinetic Processing,” Science 250(4984):1135-9 (1990); Janda et al., “Direct Selection for a Catalytic Mechanism From Combinatorial Antibody Libraries,” Proc Nat'l Acad Sci USA 91(7):2532-6 (1994); and Wirsching et al., “An Unexpectedly Efficient Catalytic Antibody Operating by Ping-pong and Induced Fit Mechanisms,” Science 252(5006):680-5 (1991), which are hereby incorporated by reference in their entirety) that accelerate cis/trans isomerization of the pSer/Thr-Pro motif. Enzymes accelerate reactions by binding to the transition state better than they bind to the substrate (or product). If a stable analog of a transition state for a given reaction is used as an antigen, the resulting antibodies will potentially catalyze the given reaction. Basically, by raising antibodies that bind tightly to a stable transition state analog, the resulting binding site is expected to replicate features of the natural enzyme such that it binds tightly to the transition state itself, and catalyzes the corresponding substrate-to-product reaction. Thus, exemplary isomerization catalysts include, e.g., catalytic antibodies raised against the transition state between the cis and trans conformations of the pSer/Thr-Pro motif.

By way of example, the generation of catalytic antibodies with peptidyl-prolyl cis/trans isomerase activity are described in Yli-Kauhaluoma et al., “Catalytic Antibodies with Peptidyl-prolyl Cis-trans Isomerase Activity,” J Am Chem Socy 118:5496-5497 (1996), which is hereby incorporated by reference in its entirety. The hapten used, hapten (1) (Yli-Kauhaluoma et al., “Catalytic Antibodies with Peptidyl-prolyl Cis-trans Isomerase Activity,” Am Chem Socy 118:5496-5497 (1996), which is hereby incorporated by reference in its entirety), 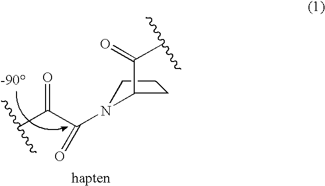
is a twisted-amide mimetic containing a dicarbonyl moiety, meant to mimic the perpendicular conformation of the peptide bond midway between the cis and trans conformations (i.e., the putative transition state of the cis-trans isomerization reaction). To generate catalytic antibodies specific to for the pSer/Thr-Pro motif of the APP, the core structure of hapten (1) could be preceded by residues V663 to pThr668 of the APP695 sequence (covalently attached at the left end of hapten (1), with the first shown carbonyl corresponding to the carbonyl group of pThr668), and followed by residues Glu670 to L674 of the APP sequence (covalently attached at the upper right end of hapten (1), beginning with the amide of Glu670).

Suitable isomerization catalysts also include RNA aptamers that bind tightly to a transition state analog. SELEX is a well established method for iteratively selecting RNA or DNA molecules from a large library of random sequences that bind and/or catalyze a specific target molecule immobilized on an affinity column (Lee et al, “Structure-function Investigation of a Deoxyribozyme with Dual Chelatase and Peroxidate Activities,” Pure Appl Chem 76:1537-45 (2004), which is hereby incorporated by reference in its entirety). This method can be used to select RNA molecules that bind to a transition state analog of the cis/trans isomerization reaction. Although the chemical repertoire of RNA is more limited than that of proteins, and some attempts to generate catalytic RNAs have not been successful (Morris et al., “Enrichment for RNA Molecules that Bind a Diels-Alder Transition State Analog,” Proc Nat'l Acad Sci USA 91(26):13028-32 (1994), which is hereby incorporated by reference in its entirety), RNA does catalyze the peptide bond during ribosomal protein synthesis (Nissen et al., “The Structural Basis of Ribosome Activity in Peptide Bond Synthesis,” Science 289(5481):920-30 (2000), which is hereby incorporated by reference in its entirety), and RNA has been shown to bind to a pThr-Pro motif (Borchers et al., “Combined Top-down and Bottom-up Proteomics Identifies a Phosphorylation Site in Stem-loop-binding Proteins that Contributes to High-affinity RNA Binding,” Proc Nat'l Acad Sci USA 103(9):3094-9 (2006), which is hereby incorporated by reference in its entirety). The RNA binding and processing domain of the stem-loop-binding protein contains a conserved motif, TPNK (SEQ ID NO: 2), in which phosphorylation of the Thr in this motif significantly contributes to RNA binding (Borchers et al., “Combined Top-down and Bottom-up Proteomics Identifies a Phosphorylation Site in Stem-loop-binding Proteins that Contributes to High-affinity RNA Binding,” Proc Nat'l Acad Sci USA 103(9):3094-9 (2006), which is hereby incorporated by reference in its entirety). Hence, RNA (in this case, a stem-loop structure in the 3′ end of histone mRNA) has evolved to bind a pThr-Pro motif. This indicates that the SELEX method using a stable transition state analog of a pSer/Thr-Pro motif of APP may be used to identify RNA aptamers that function as isomerization catalysts according to the present invention. For example, the standard SELEX method could be applied using as an affinity agent the transition state analog based on hapten (1) described above for the generation of catalytic antibodies.

The amyloid precursor protein according to this and all aspects of the present invention refers to both normal and mutant forms of the protein. For example, this aspect of the present invention may be used to restore normal cis/trans isomerization resulting from a defective APP. Preferably, the APP is a human APP, e.g., GenBank Accession Nos. NP_{—}958817 (human APP 695 aa) and NP_{—}958816 (human APP 751 aa) (Hendriks et al., “Presenile Dementia and Cerebral Haemorrhage Linked to a Mutation at Codon 692 of the β-Amyloid Precursor Protein Gene,” Nat Genet. 1(3):218-21 (1992); Jones et al., “Mutation in Codon 713 of the β Amyloid Precursor Protein Gene Presenting with Schizophrenia,” Nat Genet. 1(4):306-9 (1992); Mullan, “A Pathogenic Mutation for Probable Alzheimer's Disease in the APP Gene at the N-terminus of β-Amyloid,” Nat Genet. 1(5):345-7 (1992); Kamino et al., “Linkage and Mutational Analysis of Familial Alzheimer Disease Kindreds for the APP Gene Region,” Am J Hum Genet. 51(5):998-1014 (1992), which are hereby incorporated by reference in their entirety). An exemplary mutant form is APP^{KM670/671NL} (Hsiao et al., “Correlative Memory Deficits, Aβ Elevation, and Amyloid Plaques in Transgenic Mice,” Science 274(5284):99-102 (1996), which is hereby incorporated by reference in its entirety. The APP may be modified by natural (e.g., phosphoSer, Glu, Asp) or non-natural amino acid substitutions that place a negative charge at the T668 position, such as the human T668E mutant of APP which was shown to inhibit neurite outgrowth (Ando et al., “Role of Phosphorylation of Alzheimer's Amyloid Precursor Protein During Neuronal Differentiation,” J Neurosci 19(11):4421-7 (1999), which is hereby incorporated by reference in its entirety).

In this aspect of the present invention, isomerization of any pSer/Thr-Pro motif may be accelerated. In a preferred embodiment, the pSer/Thr-Pro motif is a pThr668-Pro motif.

Inhibition of amyloidogenic processing of the APP according to this aspect of the present invention includes any decrease in the rate or level of amyloidogenic processing.

This aspect of the present invention may be carried out in vitro or in vivo.

This aspect of the present invention may be carried out, e.g., in a cell. Suitable cells include, for example, mammalian cells, preferably human, mouse, or hamster cells. When carried out in vitro, preferred cells include, without limitation, brain cells, neuronal cells, human N18 neuroblastoma cells, H4 neuroglioma cells, human embryonic kidney 293 cells, mouse Pin1 knockout breast cancer cells, and Chinese hamster ovary cells. When carried out in vivo, preferred cells include, without limitation, human cells or mouse cells, e.g., brain cells, neuronal cells, cells of Pin1 knockout mice, and cells of APP mutant transgenic mice).

The present invention also relates to methods of inhibiting production of Aβ peptides by a cell.

Inhibition of Aβ production includes any reduction in the rate or level of Aβ production.

Suitable Aβ peptides according to these aspects of the present invention include, without limitation, A042 (e.g., residues D672-A713 of GenBank Accession Number NP_{—}958816 and residues D616-A657 of GenBank Accession Number NP_{—}958817) and Aβ40 (e.g., residues D672-V711 of GenBank Accession Number NP_{—}958816 and residues D616-V655 of GenBank Accession Number NP_{—}958817). Preferably, production of Aβ42 is inhibited.

Inhibition of Aβ production according to these aspects of the present invention may be carried out in vitro or in vivo.

Suitable cells include, for example, mammalian cells, preferably human, mouse, or hamster cells. When carried out in vitro, preferred cells include, without limitation, brain cells, neuronal cells, human N18 neuroblastoma cells, H4 neuroglioma cells, human embryonic kidney 293 cells, mouse Pin1 knockout breast cancer cells, and Chinese hamster ovary cells. When carried out in vivo, preferred cells include, without limitation, human cells or mouse cells, e.g., brain cells, neuronal cells, cells of Pin1 knockout mice, and cells of APP mutant transgenic mice).

In one aspect of the present invention, Aβ production is inhibited by contacting the cell with a compound that mimics the cis conformation of a pSer/Thr-Pro motif of an APP under conditions effective to inhibit production of Aβ peptides by the cell.

Without being bound by theory, it is expected that compounds mimicking the cis conformation of a pSer/Thr-Pro motif compete with APP for binding with agents involved in amyloidogenic processing of APP, thereby inhibiting Aβ production. Using these compounds in cells having an abnormally high level of APP containing a cis-pSer/Thr-Pro motif (“cis-pSer/Thr-Pro-APP”) could inhibit amyloidogenic APP processing/Aβ production despite the accumulation of cis-p/Thr-Pro-APP. Such cells include, for example, cells in which cis/trans isomerization is reduced, e.g., due to absence of or reduction in functional Pin1, or presence of a form of APP that resists isomerization; cells in which the net cis:trans ratio is >10:<90; cells characterized by overproduction of APP; and/or cells in which the amount of APP phosphorylated at T668 is increased.

In this aspect of the present invention, the compound may mimic the cis conformation of any pSer/Thr-Pro motif. In a preferred embodiment, the pSer/Thr-Pro motif is a pThr668-Pro motif.

Suitable compounds that mimic the cis conformation of a pSer/Thr-Pro motif of APP according to this and all aspects of the present invention include, without limitation, a compound of formula 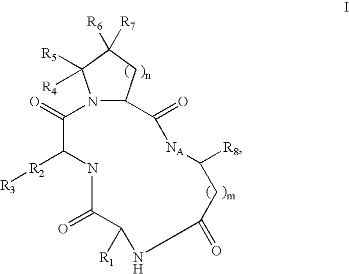
where R₁ is an amino acid-based side chain; R₂ is a glutamic acid-based side chain, an aspartic acid-based side chain, or a moiety of the formula -Ser/Thr-X—Y₍₂₎, where Ser/Thr is a serine amino acid-based side chain or a threonine amino acid-based side chain, X is a negatively charged tetra- or penta-valent moiety selected from the group consisting of —OPO₃²⁻, PO₃²⁻, —OSO₃²⁻, and —OBO₂²⁻, and Y is independently hydrogen, a blocking group, or absent; R₃ is absent or a linker between R₂ and N_{A}; R₄ and R₅ are independently hydrogen or C₁₋₃ alkyl; R₆ and R₇ are independently hydrogen or halogen; R₈ is —COR where R is a peptide of 0 to approximately 40 amino acid units; m is 1 or 2; n is 1, 2, or 3; and R₁ and/or R₈ are optionally modified to facilitate transport and/or cellular uptake of the compound and/or attachment of the compound to a substrate; and where the compound mimics the cis conformation of a pSer/Thr-Pro motif of an APP.

Amino acid side chains according to this and all aspects of the present invention can be any amino acid side chain—from natural or nonnatural amino acids—including alpha amino acids, beta amino acids, gamma amino acids, L-amino acids, D-amino acids, and N-methyl amino acids. As used herein, amino acid side chains includes analogs thereof. In general, an amino acid analog includes substitution with or addition of methyl, hydroxyl, fluorinine, bromine, etc., in the side chain. For example, threonine analog β-hydroxynorvaline (Hortin et al., “Inhibition of Asparagine-linked Glycosylation by Incorporation of a Threonine Analog into Nascent Peptide Chains,” J Biol Chem 255(17):8007-10 (1980), which is hereby incorporated by reference in its entirety), serine analog α-aminoisobutyrate (Noall et al., “Endocrine Control of Amino Acid Transfer; Distribution of an Unmetabolizable Amino Acid,” Science 126:1002-5 (1957), which is hereby incorporated by reference in its entirety); glutamate analogs kainic acid (Artuso F. et al., “Kainic Acid as Conformationally Constrained Glutamic Acid Analog in Peptide Synthesis,” Tetrahedron Lett 36(51):9309-12 (1995), which is hereby incorporated by reference in its entirety), cycloglutamic acid (Gass & Meister, “1-Amino-1,3-Dicarboxycyclohexane (Cycloglutamic Acid), a New Glutamic Acid Analog and a Substrate of Glutamine Synthetase,” Biochem 9(4):842-6 (1970), which is hereby incorporated by reference in its entirety), or synthetic caged compound analogs of glutamic acid that are photo-activated by ultraviolet light for potential use in studying the dynamics of cellular recognition processes (Corrie et al., “Postsynaptic Activation at the Squid Giant Synapse by Photolytic Release of L-Glutamate from a ‘caged’ L-glutamate,” J Physiol 465:1-8 (1993), which is hereby incorporated by reference in its entirety); and the phosphonic analog of aspartic acid (Budyin et al., “Effect of Aspartic Acid Derivatives, N-Acetyl-aspartate and its Phosphonic Analog PIR-87-6-0, on Dopamine Release from the Rat Striatum During Perfusion in Vitro,” Biull Eksp Biol Med 123(1):57-60 (1997), which is hereby incorporated by reference in its entirety).

R₁ of formula I is an amino acid side chain. Exemplary amino acid side chains include, without limitation, a valine amino acid-based side chain (the residue N-terminal to Thr668 in the natural APP), and a cysteine amino acid-based side chain, which can be used for chemical modification of the compound at this position, such as for attachment of a group for immobilizing the compound on a substrate or for facilitating its transport to a target and/or its uptake into cells.

R₂ of formula I is a glutamic acid-based side chain, an aspartic acid-based side chain, or a moiety of the formula -Ser/Thr-X—Y₍₂₎, where Ser/Thr is a serine amino acid-based side chain or a threonine amino acid-based side chain, X is a negatively charged tetra- or penta-valent moiety selected from the group consisting of —OPO₃²⁻, —PO₃²⁻, —OSO₃²⁻, and —OBO₂²⁻, and Y is independently hydrogen, a blocking group, or absent.

As will be appreciated by one of ordinary skill in the art, glutamic acid and aspartic acid mimic the chemical properties of phosphorylated Ser/Thr residues and, therefore, are expected to be functional mimics of pSer/Thr. Negative charges can hinder the transport of compounds across cell membranes. To overcome this effect, blocking groups may be added to one or more O⁻ atoms of the negatively charged moiety according to this and all aspects of the present invention. As will be appreciated by those skilled in the art, blocking groups are groups that facilitate uptake of negatively-charged moieties into cells. Exemplary blocking groups include, e.g., (CH₃)₃CCOOCH₂— and (CH₃)₃CCOS(CH₂)₂—, which groups have been successfully used in the delivery of nucleic acid-based prodrugs into cells and are biotransformed into inactive by-products once inside the target cell (Khan et al., “Bis(pivaloyloxymethyl) Thymidine 5′-Phosphate Is a Cell Membrane-permeable Precursor of Thymidine 5′-Phosphate in Thymidine Kinase Deficient CCRF CEM Cells,” Biochem Pharmacol 69(9):1307-13 (2005); Rose et al., “Bis(tBuSATE) Phosphotriester Prodrugs of 8-Azaguanosine and 6-Methylpurine Riboside; Bis(pom) Phosphotriester Prodrugs of 2′-Deoxy-4′-thioadenosine and its Corresponding 9a Anomer,” Nucleosides Nucleotides Nucleic Acids 24(5-7):809-13 (2005), which are hereby incorporated by reference in their entirety).

R₃ of formula I is absent or a linker that serves to restrict the position of the R₂ moiety through formation of a covalent bond between R₂ and N_{A}. Exemplary linkers include a moiety of formula -(A)ₙ-Z, where A is C, N, O, S, or absent, preferably C, and Z is a covalent bond between R₃ and N_{A}. Where R₂ is a moiety of the formula -Ser/Thr-X—Y₍₂₎, the linker is preferably attached via one of the oxygen atoms of the negatively charged moiety, replacing a Y. Where R₂ is a glutamic acid-based side chain or an aspartic acid-based side chain, the linker is preferably attached via one of the oxygen atoms of the negatively charged moiety.

R₄ and R₅ of formula I are independently hydrogen or C₁₋₃ alkyl, e.g., methyl. Preferably, R₄ and R₅ are both methyl. As will be apparent to one of ordinary skill, the heterocyclic ring of formula I mimics a proline amino acid side chain. Alkylation (especially methylation) at R₄ and R₅ mimics dimethyl-proline, which is expected to increase the preference for the cis conformation.

R₆ and R₇ of formula I are independently hydrogen or halogen, e.g., fluorine, chlorine, or bromine. Halogenation influences the cis/trans preference of the compound. Halogenation at R₆ (specified as the 4R position) increases the trans conformation, while halogenation at R₇ (specified as the 4S position) increases the preference for the cis conformation. In preferred embodiments according to this aspect of the present invention, R₆ is hydrogen and R₇ is fluorine.

The ring to which R₄-R₇ are attached is a 5- to 7-membered substituted or unsubstituted heterocyclic group (n is 1, 2, or 3). Based on the other constituents in the compound, the ring size may be adjusted to increase the propensity for adopting the cis conformation at this position.

R₈ of formula I is —COR where R is a peptide of 0 to approximately 40 amino acids, preferably 8 to 43 amino acids, most preferably 26 to 43 amino acids. Suitable examples of R include, without limitation, residues E671 through N695 of APP695 (GenBank Accession No. NP_{—}958817, which is hereby incorporated by reference in its entirety), residues E671 through M677 of APP695 (GenBank No. NP_{—}958817, which is hereby incorporated by reference in its entirety), -ERHLSKMQQC (SEQ ID NO: 3), and -ERHLSKMQQNGYENPTYKFFEQMQNC (SEQ ID NO: 4), optionally including an affinity agent (e.g., biotin or a peptide-based affinity tag, e.g., 6-His), a Cys-containing sequence for covalent modification (preferably at the C-terminal end), and/or an internalization sequence (e.g., YARAAARQARA (SEQ ID NO: 5) (Ho et al., “Synthetic Protein Transduction Domains: Enhanced Transduction Potential in Vitro and in Vivo,” Cancer Res 61(2):474-7 (2001), which is hereby incorporated by reference in its entirety)). For example, suitable embodiments of R also include -ERHLSKMQQNGYENPTYKFFEQMQNYARAAARQARA (SEQ ID NO: 6) and -ERHLSKMQQYARAAARQARA (SEQ ID NO: 7).

R₁ and/or R₈ of formula I may be modified to facilitate transport and/or cellular uptake of the compound and/or attachment of the compound to a substrate, as will be apparent to one of ordinary skill. For example, R₁ and/or R₈ may be covalently attached to an affinity agent (e.g., biotin or a peptide-based affinity tag, e.g., 6-His) or an internalization sequence (e.g., YARAAARQARA (SEQ ID NO: 5) (Ho et al., “Synthetic Protein Transduction Domains Enhanced Transduction Potential in Vitro and in Vivo,” Cancer Res 61(2):474-7 (2001), which is hereby incorporated by reference in its entirety) to facilitate its uptake by a cell. Other modifications include the addition of agents that facilitate transport of the compound to a target cell (e.g., neuron), organ (e.g., brain), and/or tissue (e.g., brain tissue), including an agent that facilitates its transport across the blood-brain barrier.

Suitable compounds that mimic the cis conformation of a pSer/Thr-Pro motif of amyloid precursor protein according to this and all aspects of the present invention also include, without limitation, a compound of formula 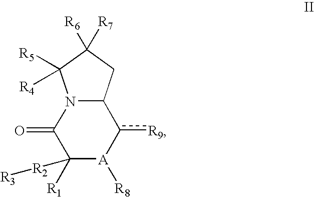
where R₁ is —H or —NHR₃ where Rₐ is a peptide of 0 to approximately 40 amino acid units; R₂ is a glutamic acid-based side chain, an aspartic acid-based side chain, or a moiety of the formula -Ser/Thr-X—Y₍₂₎, where Ser/Thr is a serine amino acid-based side chain or a threonine amino acid-based side chain, X is a negatively charged tetra- or penta-valent moiety selected from the group consisting of OPO₃²⁻, —PO₃²⁻, —OSO₃²⁻, and —OBO₂²⁻, and Y is independently hydrogen, a blocking group, or absent; R₃ is absent or a linker between R₂ and A; R₄ and R₅ are independently hydrogen or C₁₋₃ alkyl; R₆ and R₇ are independently hydrogen or halogen; R₈ is —H or —CH(CH₂)₂COOHCOR_{b} where R_{b} is a peptide of 0 to approximately 40 amino acid units; R₉ is a hydrogen bond acceptor; A is N, O, C, or S; [symbol] is a single or double bond; and R₁ and/or R₈ are optionally modified to facilitate transport and/or cellular uptake of the compound and/or attachment of the compound to a substrate; and where the compound mimics the cis conformation of a pSer/Thr-Pro motif of an APP.

R₁ of formula II is —H or —NHRₐ where Rₐ is a peptide of 0 to approximately 40 amino acid units, preferably 6 to 37 amino acids, most preferably 20 to 37 amino acids. Suitable examples of Rₐ include, without limitation, residues K649 through V667 of APP and residues V663 through V667 of APP, optionally including an affinity agent (e.g., biotin or a peptide-based affinity tag, e.g., 6-His), a Cys-containing sequence for covalent modification (preferably at the N-terminus) and/or an internalization sequence (e.g., YARAAARQARA (SEQ ID NO: 5) (Ho et al., “Synthetic Protein Transduction Domains: Enhanced Transduction Potential in Vitro and in Vivo,” Cancer Res 61(2):474-7 (2001), which is hereby incorporated by reference in its entirety)).

R₂ of formula II is a glutamic acid-based side chain, an aspartic acid-based side chain, or a moiety of the formula -Ser/Thr-X—Y₍₂₎, where Ser/Thr is a serine amino acid-based side chain or a threonine amino acid-based side chain, X is a negatively charged tetra- or penta-valent moiety selected from the group consisting of —OPO₃²⁻, —PO₃²⁻, —OSO₃²⁻, and —OBO₂²⁻, and Y is independently hydrogen, a blocking group, or absent.

R₃ of formula II is absent or a linker that serves to restrict the position of the R₂ moiety through formation of a covalent bond between R₂ and A. Exemplary linkers include a moiety of formula -(A′)ₙ-Z, where A′ is C, N, O, S, or absent, preferably C, and Z is a covalent bond between R₃ and A. Where R₂ is a moiety of the formula -Ser/Thr-X—Y₍₂₎, the linker is preferably attached via one of the oxygen atoms of the negatively charged moiety, replacing a Y. Where R₂ is a glutamic acid-based side chain or an aspartic acid-based side chain, the linker is preferably attached via one of the oxygen atoms of the negatively charged moiety.

R₄ and R₅ of formula II are independently hydrogen or C₁₋₃ alkyl, e.g., methyl. Preferably, R₄ and R₅ are both hydrogen. As will be apparent to one of ordinary skill, the pentacyclic ring of formula II mimics a proline amino acid side chain. Alkylation (especially methylation) at R₄ and R₅ mimics dimethyl-proline, which is expected to be compatible with the locked cis conformation of this compound and provides additional hydrophobic character to the compound.

R₆ and R₇ of formula II are independently hydrogen or halogen, e.g., fluorine, chlorine, or bromine (preferably fluorine). Halogenation influences the cis/trans preference of the compound. Halogenation at R₆ (specified as the 4R position) favors the C^{γ}-exo ring conformation, while halogenation at R₇ (specified as the 4S position) favors the C^{γ}-endo ring conformation. In preferred embodiments according to this aspect of the present invention, R₆ and R₇ can be selected to enrich the population of either ring conformation, as will be apparent to the skilled artisan.

R₈ of formula II is —H or —CH(CH₂)₂COOHCOR_{b} where R_{b} is a peptide of 0 to approximately 40 amino acids, preferably 8 to 43 amino acids, most preferably 26 to 43 amino acids. Suitable examples of R_{b} include, without limitation, residues E671 through N695 of APP695 (GenBank Accession No. NP_{—}958817, which is hereby incorporated by reference in its entirety), residues E671 through M677 of APP695 (GenBank No. NP_{—}958817, which is hereby incorporated by reference in its entirety), -ERHLSKMQQC (SEQ ID NO: 3), and -ERHLSKMQQNGYENPTYKFFEQMQNC (SEQ ID NO: 4), optionally including an affinity agent (e.g., biotin or a peptide-based affinity tag, e.g., 6-His), a Cys-containing sequence for covalent modification (preferably at the C-terminal end), and/or an internalization sequence (e.g., YARAAARQARA (SEQ ID NO: 5) (Ho et al., “Synthetic Protein Transduction Domains: Enhanced Transduction Potential in Vitro and in Vivo,” Cancer Res 61(2):474-7 (2001), which is hereby incorporated by reference in its entirety)). For example, suitable embodiments of R_{b} also include -ERHLSKMQQNGYENPTYKFFEQMQNYARAAARQARA (SEQ ID NO: 6) and -ERHLSKMQQYARAAARQARA (SEQ ID NO: 7).

R₉ of formula II is a hydrogen bond acceptor, preferably a hydroxyl or carbonyl.

A of formula II is an atom that is stable within the ring structure, preferably N, O, C, or S, most preferably N, O, or C.

R₁ and/or R₈ of formula II may be modified to facilitate transport and/or cellular uptake of the compound and/or attachment of the compound to a substrate, as noted above with respect to formula I.

Compounds of formula I are expected to exist in a cis:trans ratio of >10:<90. Compounds according to formula II are expected to be virtually 100% cis.

Exemplary compounds according to this aspect of the present invention include, without limitation, those set forth in Table 1.

TABLE 1
Exemplary cis-pSer/Thr-Pro Mimics.

Formula I

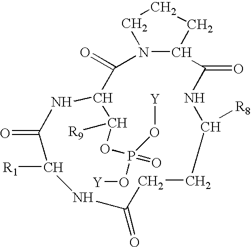

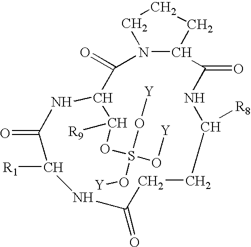

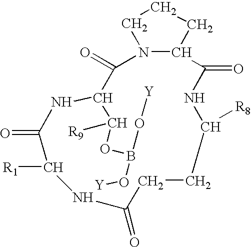

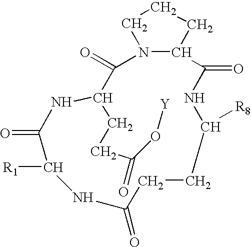

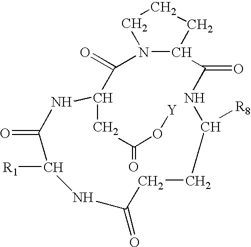

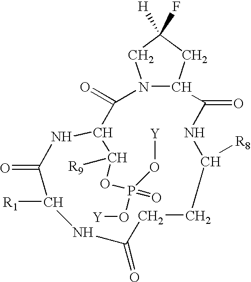

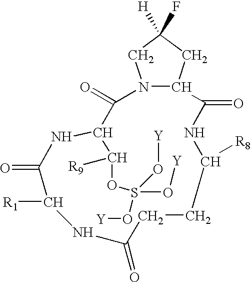

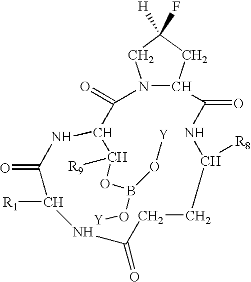

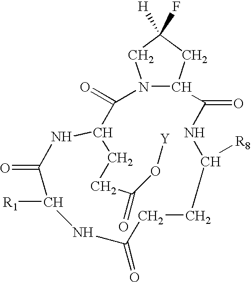

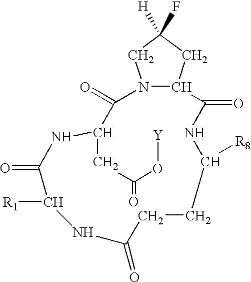

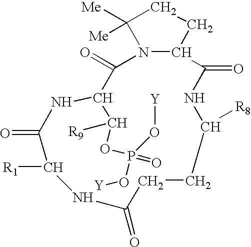

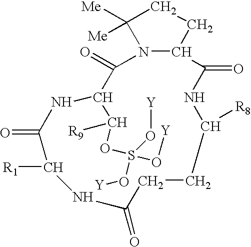

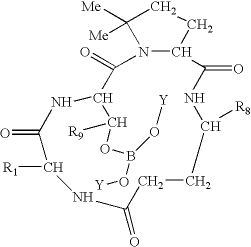

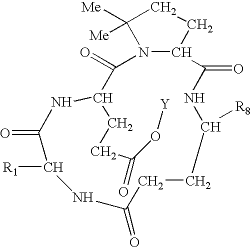

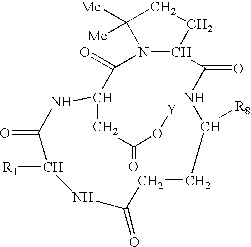

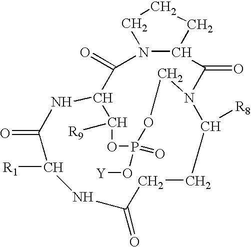

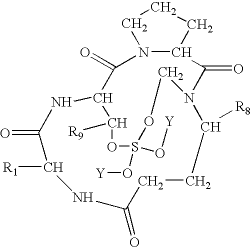

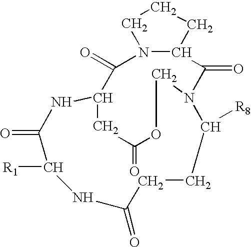

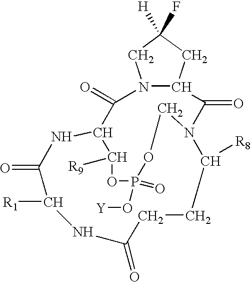

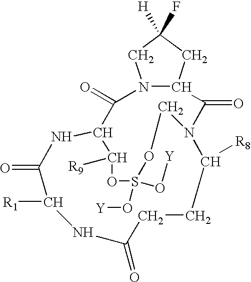

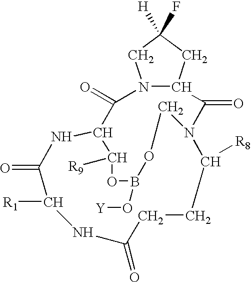

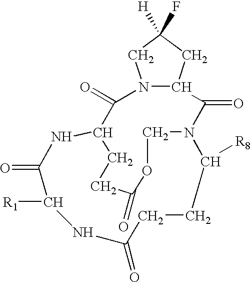

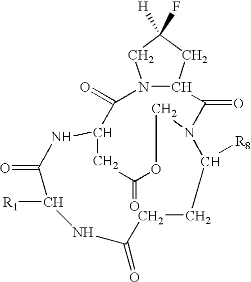

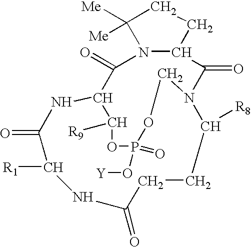

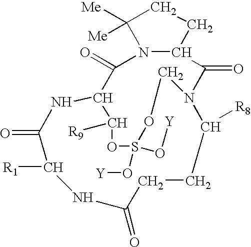

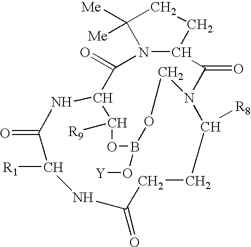

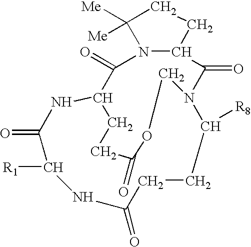

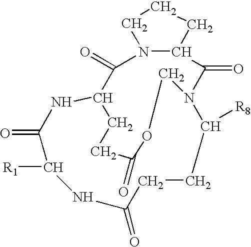

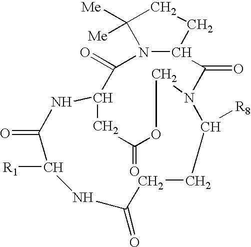

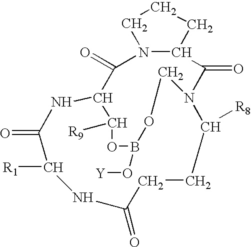

In all structures, all stereoisomers are implied unless explicitly identified
R_{1 = —CH(CH}₃)₂ or —CH₂SH
Y = absent or (CH₃)₃CCOOCH₂—
R₈ = —COR, where R = -ERHLSKMQQC (SEQ ID NO: 3),
-ERHLSKMQQNGYENPTYKFFEQMQNC (SEQ ID NO: 4),
-ERHLSKMQQNGYENPTYKFFEQMQNYARAAARQARA
(SEQ ID NO: 6), or -ERHLSKMQQYARAAARQARA (SEQ ID NO: 7)
R₉ = H or CH₃

Formula II

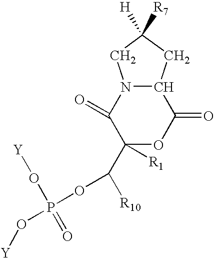

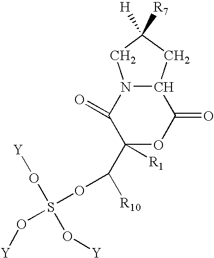

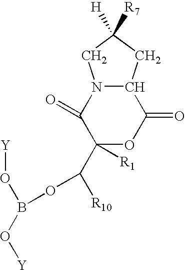

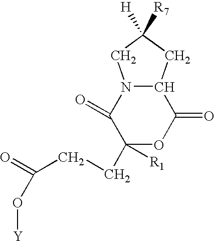

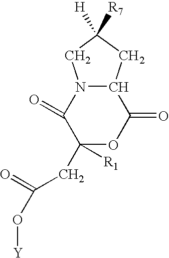

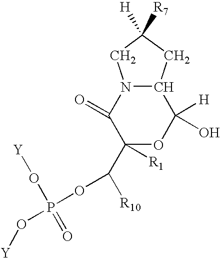

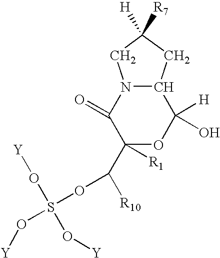

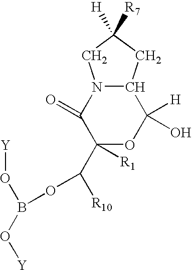

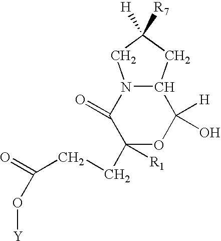

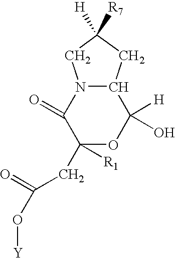

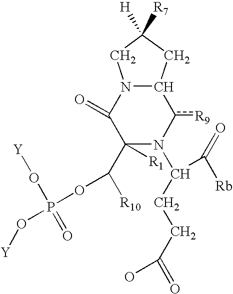

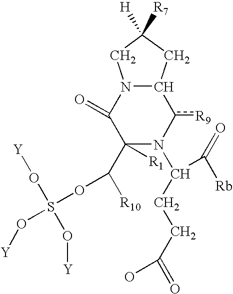

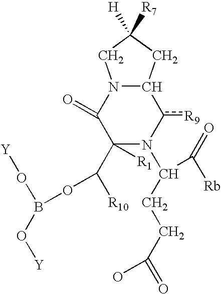

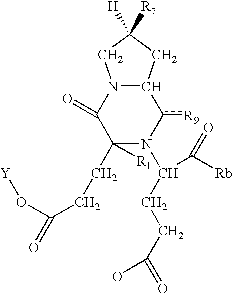

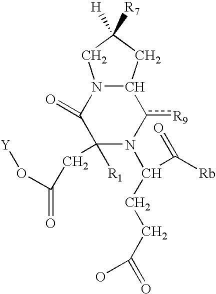

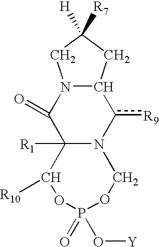

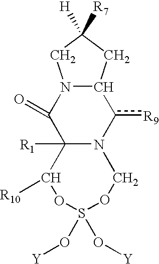

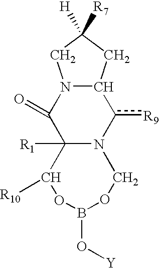

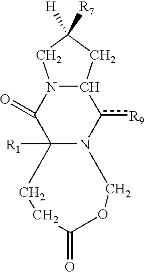

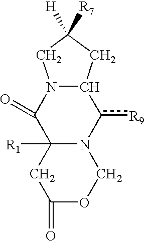

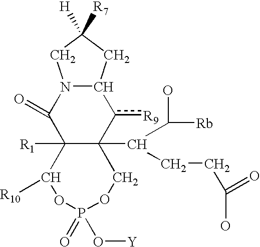

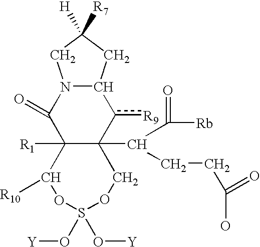

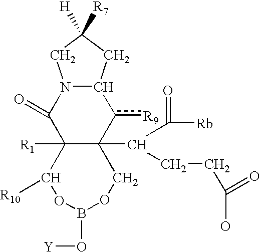

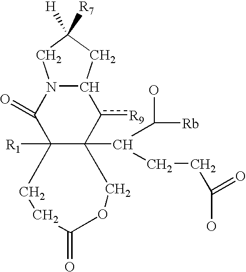

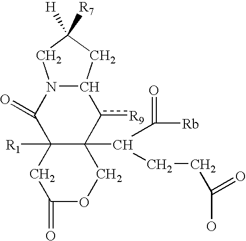

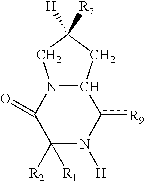

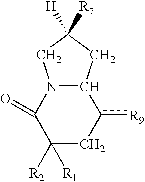

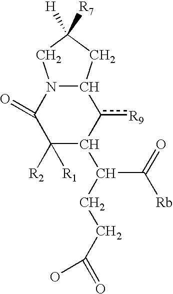

In all structures, all stereoisomers are implied unless explicitly identified
R₁ = —H or —NHRₐ
Rₐ = CVDAAV-(SEQ ID NO: 8),
YARAAARQARAVDAAV-(SEQ ID NO: 9),
YARAAARQARAKKKQYTSIHHGVVEVDAAV-(SEQ ID NO: 10),
or CKKKQYTSIHHGVVEVDAAV-(SEQ ID NO: 11)
R₂ is a glutamic acid-based side chain, an aspartic acid-based side chain,
or a moiety of the formula -Ser/Thr-X-Y₍₂₎,
where Ser/Thr is a serine amino acid-based side chain or a
threonine amino acid-based side chain, X is a negatively
charged tetra- or penta-valent moiety selected from the group
consisting of —OPO₃²⁻, —PO₃²⁻, —OSO₃²⁻, and —OBO₂²⁻
Y = absent or (CH₃)₃CCOOCH₂—
R₇ = H or F
R₉ = O or OH
R₁₀ = H or CH₃
R_{b} = -ERHLSKMQQC (SEQ ID NO: 3),
-ERHLSKMQQNGYENPTYKFFEQMQNC (SEQ ID NO: 4),
-ERHLSKMQQNGYENPTYKFFEQMQNYARAAARQARA (SEQ ID
NO: 6), or -ERHLSKMQQYARAAARQARA (SEQ ID NO: 7)

In another aspect of the present invention Aβ production is inhibited by accelerating cis/trans isomerization of APP at a pSer/Thr-Pro motif under conditions effective to inhibit production of Aβ peptides by the cell.

Acceleration may be carried out by contacting the pSer/Thr-Pro motif with an isomerization catalyst, as described above. Suitable catalysts include, for example, Pin1, Pin1 homologues, and isomerization catalysts based on the transition state analog of the APP pSer/Thr-Pro motif (e.g., catalytic antibodies and RNA aptamers).

Preferably, the APP according to this aspect of the present invention is a human APP, e.g., GenBank Accession Nos. NP_{—}958817 (human APP 695 aa), NP_{—}958816 (human APP 751 aa), or familial APP mutants, including those identified in Hardy & Crook, “APP Mutations Table,” Alzheimer Research Forum (2005), which is hereby incorporated by reference in its entirety.

In this aspect of the present invention, isomerization of any pSer/Thr-Pro motif may be accelerated. In a preferred embodiment, the pSer/Thr-Pro motif is a pThr668-Pro motif.

Another aspect of the present invention relates to a method of treating and/or preventing in a subject a degenerative neurological disease characterized by amyloidogenic processing of amyloid precursor protein and/or overproduction of amyloid beta peptide. This method involves administering to the subject an agent that (1) accelerates cis/trans isomerization of APP at a pSer/Thr-Pro motif and/or (2) inhibits production of Aβ peptides, under conditions effective to treat and/or prevent the disease in the subject.

According to this and all aspects of the present invention, degenerative neurological diseases characterized by amyloidogenic processing of amyloid precursor protein and/or overproduction of amyloid beta peptide include Alzheimer's Disease.

Preferably, the method according to this aspect of the present invention is carried out to treat a mammalian subject, most preferably a human subject. Other suitable subjects include, without limitation transgenic mice, e.g., mice overexpressing mutant APP or presenilin and/or tau mutants.

Suitable agents according to this aspect of the present invention include, without limitation, isomerization catalysts described above (e.g., Pin1, Pin1 homologues, and catalysts based on the transition state analog of the APP pSer/Thr-Pro motif, for example catalytic antibodies and RNA aptamers), and compounds that mimic the cis conformation of a pSer/Thr-Pro motif of an APP (e.g., compounds according to formulae I and II described above).

As will be apparent to one of ordinary skill in the art, the agent may be administered using generally known methods. Typically, the agent is administered by introducing the agent into the subject. In some embodiments, for example when a polypeptide agent (e.g., Pin1) is used, the agent may be administered by introducing into the subject a nucleic acid molecule that encodes the polypeptide (JOSEPH SAMBROOK & DAVID W. RUSSELL, 1 MOLECULAR CLONING: A LABORATORY MANUAL (3d ed. 2001), SHORT PROTOCOLS IN MOLECULAR BIOLOGY (Frederick M. Ausubel et al. eds., 1999), and U.S. Pat. No. 4,237,224 to Cohen and Boyer, which are hereby incorporated by reference in their entirety).

Agents according to this aspect of the present invention may be administered orally or parenterally (e.g., intradermally, subcutaneously, intravenously, intramuscularly, intraperitoneally, by intranasal instillation, by intravesical instillation, intracavitarily, intraocularly, intraarterially, intralesionally, or by application to mucous membranes, such as that of the nose, throat, and bronchial tubes). Exemplary delivery devices include, without limitation, liposomes, transdermal patches, implants, syringes, and gene therapy. They may be administered alone or with suitable pharmaceutical carriers, and can be in solid or liquid form, such as tablets, capsules, powders, solutions, suspensions, or emulsions.

The agents may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of the agent. The percentage of the agent in these compositions may, of course, be varied and may conveniently be between about 2% to about 60% of the weight of the unit. The amount of the agent in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The agents may also be administered parenterally. Solutions or suspensions of the agent can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The agents according to this aspect of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The agents of the present invention may be administered directly to the targeted tissue. Additionally and/or alternatively, the agent may be administered to a non-targeted area along with one or more agents that facilitate migration of the agent to (and/or uptake by) a targeted tissue, organ, or cell. Preferred targeted tissues include neuronal tissue. Preferred targeted organs include the brain. Preferred targeted cells include neurons and neuroblastoma cells. As will be apparent to one of ordinary skill in the art, the agent according to this aspect of the present invention may itself be modified to facilitate its transport to (and uptake by) the desired tissue, organ, or cell. For example, compounds of formula I or formula II can be modified as described above to facilitate their transport to a target cell (e.g., neuron), organ (e.g., brain), and/or tissue (e.g., brain tissue), including its transport across the blood-brain barrier; and/or its uptake by the target cell (e.g., its transport across cell membranes).

Exemplary delivery devices include, without limitation, liposomes, transdermal patches, implants, implantable or injectable protein depot compositions, syringes, and gene therapy. Other delivery systems which are known to those of skill in the art can also be employed to achieve the desired delivery of the fusion protein to the desired organ, tissue, or cells in vivo to effect this aspect of the present invention.

This aspect of the present invention is carried out under conditions effective to treat and/or prevent the degenerative neurological disease in the subject. Treatment includes any reduction in the amount of Aβ production in the subject, any reduction in the size and/or number of amyloid plaques in the brain of the subject, and/or any improvement in the loss of cognitive function associated with the disease. Prevention includes prevention of the development of amyloid plaques (or the prevention of further increase in the size and/or number of existing plaques) in the brain of the subject, and/or prevention of loss of cognitive function associated with the disease.

Yet another aspect of the present invention relates to a method of screening for a therapeutic agent effective in treating and/or preventing in a subject a degenerative neurological disease characterized by amyloidogenic processing of amyloid precursor protein and/or overproduction of amyloid beta peptide. In this aspect of the present invention, a substrate compound comprising a phosphorylated serine/threonine-proline motif of an amyloid precursor protein and a candidate compound are provided. The candidate compound is contacted with the substrate compound, and the cis/trans isomerization rate of the pSer/Thr-Pro motif in the presence of the candidate compound is measured. The cis/trans isomerization rate in the presence of the candidate compound is compared to a reference cis/trans isomerization rate, where acceleration of the cis/trans isomerization rate in the presence of the candidate compound relevant to the reference cis/trans isomerization rate indicates that the candidate compound is a potential therapeutic agent effective in treating and/or preventing the disease in a subject.

Suitable substrate compounds according to this aspect of the present invention include, for example, an amyloid precursor protein or fragment thereof. When fragments are used, the fragment preferably contains at least the sequence GVVEVDAAVpTPEERHLSKMQQ (SEQ ID NO: 12) (Ramelot & Nicholson, “Phosphorylation-induced Structural Changes in the Amyloid Precursor Protein Cytoplasmic Tail Detected by NMR,” J Mol Biol 307(3):871-84 (2001), which is hereby incorporated by reference in its entirety).

The candidate compound may be contacted with the substrate compound by any means known in the art or which may be developed hereafter.

Candidate compounds that may be screened include, without limitation, proteins, peptides, peptidomimetics, peptoids, small molecules, and other potential therapeutic agents.

The cis/trans isomerization rate may be measured using techniques that will be apparent to the skilled artisan, based on the particular experimental protocol. Exemplary assays include calorimetric assays (Schutkowski et al., “Role of Phosphorylation in Determining the Backbone Dynamics of the Serine/Threonine-Proline Motif and Pin1 Substrate Recognition,” Biochemistry 37(16):5566-75 (1998), which is hereby incorporated by reference in its entirety) and NMR-based assays (e.g., as described in Example 3).

The reference cis/trans isomerization rate can be a statistically predetermined range of rates that one would expect to be measured under a particular experimental protocol. The reference can be set at a 95%, 97%, 98%, or 99% confidence level. Alternatively, the reference cis/trans isomerization rate can be an internal control performed in parallel with the test assay of the present invention. Basically, a second substrate compound is provided, and the cis/trans isomerization rate of the pSer/Thr-Pro motif of the second substrate compound is measured under comparable conditions in the absence of the candidate compound. The cis/trans isomerization rate between the first and second pSer/Thr-Pro motifs can then be compared.

Another aspect of the present invention relates to a method of screening for a therapeutic agent effective in treating and/or preventing in a subject a degenerative neurological disease characterized by amyloidogenic processing of amyloid precursor protein and/or overproduction of amyloid beta peptide. This method involves providing a temperature sensitive Ess1/Ptf1 mutant yeast cell and contacting the cell with a candidate compound. The cell is cultured at a temperature effective to cause terminal mitotic arrest of the yeast cell due to an absence of Ess1/Ptf1 function, and whether the cell displays a temperature-sensitive phenotype during culturing is evaluated. Compounds that prevent the yeast cell from displaying the temperature-sensitive phenotype are identified as likely therapeutic agents effective in treating and/or preventing in a subject a degenerative neurological disease characterized by amyloidogenic processing of amyloid precursor protein and/or overproduction of amyloid beta peptide.

Ess1/Ptf1 is an essential protein in budding yeast. Yeast containing a mutant Ess1/Ptf1 protein (including Ess1/Ptf1 knockouts) exhibit temperature-sensitive phenotypes, including death and/or reduced growth at higher temperatures. Pin1 is structurally and functionally homologous to Ess1/Ptfl. By way of example, when driven by the endogenous ESS1 promoter, human Pin1 protein fully functions like Ess1/Ptf1 protein in yeast, rescuing the lethal phenotype of the ts ESS1 mutant strain YPM2 at the restrictive temperature (Zhou et al., “Pin1-dependent Prolyl Isomerization Regulates Dephosphorylation of Cdc25C and Tau Proteins,” Mol Cell 6:873-883 (2000); Lu et al., “A Human Peptidyl-prolyl Isomerase Essential for Regulation of Mitosis,” Nature 380(6574):544-7 (1996); Lu et al., “A Function of WW Domains as Phosphoserine- or Phosphothreonine-binding Modules,” Science 283:1325-1328 (1999), which are hereby incorporated by reference in their entirety).

Accordingly, temperature sensitive Ess1/Ptf1 mutant yeast cells can be used to screen for compounds that mimic the function of Pin1 and are likely therapeutic agents effective in treating and/or preventing in a subject a degenerative neurological disease characterized by amyloidogenic processing of amyloid precursor protein and/or overproduction of amyloid beta peptide.

Suitable candidate compounds according this aspect of the present invention include, for example, isomerization catalysts developed as described above, e.g., catalytic antibodies and RNA aptamers.

In one embodiment, by way of example, cDNA representing the catalytic antibodies or cDNA corresponding to catalytic RNA aptamers may be subcloned into a vector (e.g., vector Yep) and transformed into the temperature sensitive Ess1/Ptf1 mutant yeast cell. The transformants are grown at a permissive temperature to obtain individual stable strains and then grown in a nonpermissive temperature. If the resulting strains can grow at both the permissive and nonpermissive temperatures, it indicates that the candidate compounds in those strains can perform the essential function of Pin1. However, if the strains grow only at the permissive temperature, but not at the nonpermissive temperature, this indicates that the candidate compounds in those strains fail to perform the essential function of Pin1.

Suitable temperature sensitive Ess1/Ptf1 mutant yeast cells include, for example, the ts ESS1 mutant strain YPM2.

The cell is cultured at a temperature effective to cause terminal mitotic arrest of the yeast cell due to an absence of Ess1/Ptf1 function, which absence may be partial or complete, that is, absence includes any reduction and/or alteration in Ess1/Ptf1 function that results in a temperature sensitive phenotype. Cultering may be carried out using methods that will be apparent to the skilled artisan.

By way of example, whether the cell displays a temperature-sensitive phenotype during culturing may be evaluated by comparing its growth rate and/or survival rate to a known growth and/or survival rate for the particular temperature sensitive Ess1/Ptfl used, and/or by culturing a comparable temperature sensitive Ess1/Ptf1 mutant yeast cell under essentially the same conditions but in the absence of the candidate compound, and comparing the growth and/or survival of the two cells.

Another aspect of the present invention relates to a method of screening for biological molecules likely to be involved in the amyloidogenic pathway. In this aspect of the present invention, an APP which is phosphorylated at a Ser/Thr-Pro motif is contacted with a neuronal cell lysate, and biological molecules from the neuronal cell lysate that bind to the APP are detected. In a separate experiment, a compound that mimics the cis conformation of a pSer/Thr-Pro motif of an APP is contacted with a neuronal cell lysate, and biological molecules from the neuronal cell lysate that bind to the compound are detected, under essentially the same conditions as the experiment conducted with the APP. The binding detected in the two experiments is compared. A biological molecule which undergoes greater binding to the compound that mimics the cis conformation of a pSer/Thr-Pro motif of an APP than to the APP is likely to be involved in the amyloidogenic pathway.

Biological molecules that may be screened according to this aspect of the present invention include, without limitation, proteins, polypeptides, DNA, RNA, nucleotides, small molecules, ions, glycoproteins, polysaccharides, lipids, and glycolipids.

Preferably, the compound that mimics the cis conformation of a pSer/Thr-Pro motif of an APP has a cis:trans conformation ratio of >10:<90, most preferably 30:-70.

Preferably, the pSer/Thr-Pro motif is an pThr668-Pro motif or a derivative of the pThr668-Pro motif.

The APP and the compound that mimics the cis conformation of a pSer/Thr-Pro motif of an APP may be contacted with the neuronal cell lysate by any means known in the art or which may be developed hereafter.

Any neuronal cell lysate may be used in the aspect of the present invention, and may be prepared using methods that will be apparent to one of ordinary skill. The neuronal cell lysate may be from any neuronal cell, preferably from a brain cell. The neuronal cell is preferably a human or mouse cell.

Binding according this aspect of the present invention may be detected using any suitable method. Suitable methods include, without limitation, gel chromatography (e.g., 2D gel electrophoresis), enzyme-linked immunosorbent assay, and proteomic assay. In an exemplary embodiment, the compound includes an affinity tag (e.g., biotin or His) and the compound is immobilized on a column (e.g., streptavidin column or nickel-sepharose column). The neuronal cell lysate is passed over the column, and the column is washed to remove any weakly binding molecules. The bound biological molecules are then eluted via standard methods (e.g., a salt or pH gradient).

Biological molecules that are involved in the amyloidogenic pathway are identified by comparing binding with APP to binding with the compound that mimics the cis conformation of a phosphorylated threonine-proline motif of an amyloid precursor protein (“cis-pSer/Thr-Pro-APP mimic”). This includes biological molecules that bind to the cis-pSer/Thr-Pro-APP mimic but not to APP, as well as biological molecules that bind to both, but bind to the cis-pSer/Thr-Pro-APP mimic at higher amounts or with greater affinity.

After identifying biological molecules that are involved in the amyloidogenic pathway according to this aspect of the present invention, the biological molecules may be isolated and characterized using standard methods, including, for example, ultraviolet absorption spectrum (to identify the class of molecule), mass spectrometry, and NMR.

Another aspect of the present invention relates to a compound of formula I as defined above, where the compound mimics the cis conformation of a phosphorylated threonine-proline motif of an amyloid precursor protein.

Exemplary compounds include those set forth in Table 1.

Another aspect of the present invention relates to a compound of formula II as defined above, where the compound mimics the cis conformation of a phosphorylated threonine-proline motif of an amyloid precursor protein.

Exemplary compounds include those set forth in Table 1.

The present invention may be further illustrated by reference to the following examples.

### EXAMPLES

### Example 1

### DNA Construction, Cell Lines, Protein Expression and Purification

APP/pCMV and CHO-APP751WT cells were kind gifts of Drs. D. Goldgaber and E. Koo, respectively. APP, its mutants, and AICD (nucleotides 638-695 according to the APP695 isoform) were generated from APP/pCMV and inserted into pET28a as His-tagged proteins. To generate APP^{T668A} and AICD^{T668A} constructs, missense mutations in the APP cDNA were introduced by the QuikChange™ site-directed mutagenesis kit (Stratagene) according to manufacturer's instructions and confirmed by sequencing. Recombinant proteins were expressed and purified from bacteria or synthesized using in vitro TNT system (Lu et al., “A Function of WW Domains as Phosphoserine- or Phosphothreonine-binding Modules,” Science 283:1325-1328 (1999), which is hereby incorporated by reference in its entirety).

### Example 2

### Determination of Pin1-APP Interaction and Colocalization

APP and its mutants were phosphorylated by Xenopus mitotic extracts or by cyclin B/Cdc2 kinase (Lu et al., “The Prolyl Isomerase Pin1 Restores the Function of Alzheimer-associated Phosphorylated Tau Protein,” Nature 399:784-788 (1999), which is hereby incorporated by reference in its entirety). The interaction between Pin1 and APP or its mutants was determined using GST-Pin1 pulldown assay (Lu et al., “A Function of WW Domains as Phosphoserine- or Phosphothreonine-binding Modules,” Science 283:1325-1328 (1999), which is hereby incorporated by reference in its entirety). For competitive binding assays, synthetic peptides, APPtide (EVDAAVpTPEERHLS (SEQ ID NO: 13), and its non-phosphorylated counterpart, were synthesized and purchased from Merck Co. The accuracy and purity were identified by mass spectroscopy. APPtide was dissolved in the binding buffer described in Lu et al., “A Function of WW Domains as Phosphoserine- or Phosphothreonine-binding Modules,” Science 283:1325-1328 (1999), which is hereby incorporated by reference in its entirety, and added into the GST pull down reaction for the competitive binding assay (Lu et al., “A Function of WW Domains as Phosphoserine- or Phosphothreonine-binding Modules,” Science 283:1325-1328 (1999), which is hereby incorporated by reference in its entirety). To examine localization of Pin1 and APP, cells were fixed and stained for APP using a polyclonal antibody raised against the C-terminal domain of the protein (Sigma) or a monoclonal antibody against N-terminal APP 22C11 (Chemicon); stained for Pin1 using either anti-Pin1 monoclonal or polyclonal antibodies; and/or stained for endosomes using a monoclonal antibody for clathrin-coated vesicles (clathrin), the early endosomal antigen 1, and/or adaptor protein 1 (Transduction Laboratories) (Lu et al., “The Prolyl Isomerase Pin1 Restores the Function of Alzheimer-associated Phosphorylated Tau Protein,” Nature 399:784-788 (1999); Liou et al., “Role of the Prolyl Isomerase Pin1 in Protecting Against Age-dependent Neurodegeneration,” Nature 424:556-561 (2003); Lee et al., “APP Processing Is Regulated by Cytoplasmic Phosphorylation,” J Cell Biol 163(1):83-95 (2003), which are hereby incorporated by reference in their entirety).

### Example 3

### NMR Analysis

The pThr668-Pro peptides were synthesized, purified, and analyzed as reported in (Ramelot & Nicholson, “Phosphorylation-induced Structural Changes in the Amyloid Precursor Protein Cytoplasmic Tail Detected by NMR,” J Mol Biol 307(3):871-84 (2001), which is hereby incorporated by reference in its entirety). Briefly, for ROESY (Rotating frame Overhauser spectroscopy) experiments, peptide was dissolved in buffer (10 mM HEPES, 10 mM NaCl, 10 mM DTT, 5 mM NaN₃, 7% ²H₂O, adjusted to pH 7.0 using NaOH or HCl) and was either used directly or combined with Pin1, GST-Pin1, or its K63A mutant at 60:1 molar ratio (3 mM pThr668-Pro and 0.05 mM Pin1 or its mutant). For ¹⁵N—¹H HSQC experiments, ¹⁵N-E670-pT668 peptide was dissolved in the same buffer adjusted to pH 6.7, with subsequent addition of an equimolar amount of the WW domain (0.2 mM). All experiments were recorded at 25° C. on a Varian Inova 600 MHz spectrometer with the ¹H carrier set on water. ROESY data sets were acquired with spectral widths of 6 kHz (8 kHz) in t1 (t2) and 640 (2048) complex data points, and were processed as described in Ramelot & Nicholson, “Phosphorylation-induced Structural Changes in the Amyloid Precursor Protein Cytoplasmic Tail Detected by NMR,” J Mol Biol 307(3):871-84 (2001), which is hereby incorporated by reference in its entirety. Peak intensities were obtained using Pipp (Garrett et al., “A Common-sense Approach to Peak-picking in 2-Dimensional, 3-Dimensional, and 4-Dimensional Spectra Using Automatic Computer-analysis of Contour Diagrams,” J Magn Reson 95:214-20 (1991), which is hereby incorporated by reference in its entirety), and curve fits and error analyses were performed using SigmaPlot (Systat Software, Inc.). The ratio of cross/diagonal peak intensities for each conformation is given as described in RICHARD R. ERNST ET AL., PRINCIPLES OF NUCLEAR MAGNETIC RESONANCE IN ONE AND TWO DIMENSIONS (1987), which is hereby incorporated by reference in its entirety.
I_{ct}/I_{cc}=k_{ct}^{cat}[exp(kₑₓtₘ)−1]/[k_{tc}^{cat}exp(kₑₓtₘ)+k_{ct}^{cat}]
I_{tc}/Iₜₜ=k_{tc}^{cat}[exp(kₑₓtₘ)−1]/[k_{ct}^{cat}exp(kₑₓtₘ)+k_{tc}^{cat}]
where kₑₓ=k_{ct}^{cat}+k_{tc}^{cat}, and tₘ corresponds to the ROESY mixing time. The desired rate constants were extracted by recording ROESY spectra with different mixing times and fitting the intensity ratios obtained to the above equations. ¹⁵N—¹H HSQC data sets were acquired with spectral widths of 1.4 kHz (10 kHz) in t1 (t2) and 512 (2048) complex data points, and were processed as described in Ramelot & Nicholson, “Phosphorylation-induced Structural Changes in the Amyloid Precursor Protein Cytoplasmic Tail Detected by NMR,” J Mol Biol 307(3):871-84 (2001), which is hereby incorporated by reference in its entirety.

### Example 4

### Pin1^{-/-} and APP-TG2576 Mouse Strains

Pin1−/− mice were inbred in mixed populations of 129/Sv and C57L/B6 mice (Liou et al., “Role of the Prolyl Isomerase Pin1 in Protecting Against Age-dependent Neurodegeneration,” Nature 424:556-561 (2003), which is hereby incorporated by reference in its entirety). APP-Tg2576 mice overexpressing the human APP KM670/671NL (Swedish) mutant (Hsiao et al., “Correlative Memory Deficits, Aβ Elevation, and Amyloid Plaques in Transgenic Mice,” Science 274(5284):99-102 (1996), which is hereby incorporated by reference in its entirety) were purchased from Taconic and crossed with Pin1−/− mice to generate mice with a single copy of the APP transgene in Pin1+/+ and Pin1−/− genetic background. To avoid the possible influence of genetic backgrounds, littermates were usually used and the results were observed in multiple animals.

### Example 5

### Determination of APP Processing and Aβ Levels

APP processing was determined as described in (Pastorino et al., “BACE (3-Secretase) Modulates the Processing of APLP2 in Vivo,” Mol Cell Neurosci 25:642-49 (2004), which is hereby incorporated by reference in its entirety). Briefly, mouse brains were homogenized and centrifuged to remove debris, followed by centrifugation at 100,000×g for 40 minutes. The supernatants, representing the soluble fraction, were separated from the pellet, representing the membrane fraction. The pellet was further treated in a buffer containing 1% Triton X-100 to extract membrane-inserted proteins, and spun at 100,000×g for 40 minutes. The resulting supernatant represented the Triton X-100 membrane extracted fraction. APP full-length forms and APP C-terminal fragments in the total cell lysates and the Triton X-100 membrane-extracted fraction were assayed using C-terminal polyclonal antibodies raised against the APP intracellular domain (Sigma). In the soluble fraction total secreted APPs (αAPPs and βAPPs) was detected using monoclonal antibody (“mAb”) 22C11 (Chemicon), raised against the N-terminal domain of APP; αAPPs was detected using mAb 6E10 (Sigma) raised against amino acids 1-17 of the human Aβ peptide; and βAPPs was detected using mAb 197sw raised against the Swedish mutant form of βAPPs (kindly provided by D. Selkoe, D Schenk and P Seubert). Levels of APP full length and C-terminal fragments phosphorylated at residue T668 were detected using a pThr668-specific antibody (Biosource) (Lee et al., “APP Processing Is Regulated by Cytoplasmic Phosphorylation,” J Cell Biol 163(1):83-95 (2003), which is hereby incorporated by reference in its entirety). After blotting, levels of mature and immature APP, phosphorylated APP (full length and CTFs), soluble APPs, and CTFs were evaluated using chemiluminescence and semi-quantified using NIH Image1.63 (NIH).

Aβ40 and Aβ42 levels were measured by sandwich ELISAs in one hemisphere of each mouse at different ages (Johnson-Wood et al., “Amyloid Precursor Protein Processing and Aβ42 Deposition in a Transgenic Mouse Model of Alzheimer Disease,” Proc Nat'l Acad Sci USA 94:1550-5 (1997); Citron et al., “Mutant Presenilins of Alzheimer's Disease Increase Production of 42-Residue Amyloid β-Protein in Both Transfected Cells and Transgenic Mice,” Nat Med 3:67-72 (1997), which are hereby incorporated by reference in their entirety). Briefly, brain tissues were homogenized in a 50 mM NaCl, 0.2% DEA solution and centrifuged for 45 minutes at 100,000×g. The supernatant was neutralized by a 1/10 volume of 0.5 M Tris-HCl (pH 6.8) and used as the soluble fraction. The remaining pellets were washed with DEA buffer and then with distilled water, followed by dissolution in formic acid by sonication, and centrifugation at 130,000×g for 45 minutes. The aqueous supernatant was neutralized with a 19-fold volume neutralization buffer (1 M Tris base, 0.5 M Na2HPO4, 0.05% NaN3). In all assays, the capture antibodies 2G3 and 21F12 were used for x-40 and x-42 assays, respectively. Biotinylated 266B mAb raised against the domain spanning residues 13-28 of Aβ was used as the detecting antibody. The reporter system contained streptavidin-alkaline phosphatase and AttoPhos (Promega, Madison, Wis.) as the substrate (excitation, 450 nm; emission, 580 nm). mAbs 2G3, 21F12, and 266 were kindly provided by D Schenk and P Seubert (Elan Pharmaceuticals).

### Example 6

### Immunogold-EM

Subcellular localization of neuronal Aβ was determined using immunogold-EM (Liou et al., “Role of the Prolyl Isomerase Pin1 in Protecting Against Age-dependent Neurodegeneration,” Nature 424:556-561 (2003); Li et al., “Amino-terminal Fragments of Mutant Huntingtin Show Selective Accumulation in Striatal Neurons and Synaptic Toxicity,” Nat Genet. 25:385-389 (2000); Takahashi et al., “Intraneuronal Alzheimer A042 Accumulates in Multivesicular Bodies and Is Associated with Synaptic Pathology,” Am J Pathol 161(5): 1869-79 (2002), which are hereby incorporated by reference in their entirety). Briefly, 7-month-old mice were perfused with 3.75% acrolein (Polyscience, Pa.) and 2% paraformaldehyde, and free-floating sections from the dorsal medial cortex were labeled with anti-human Aβ42 polyclonal antibodies (Chemicon), followed by incubation with goat anti-rabbit IgG conjugated to gold particles.

### Example 7

### Interaction of Pint with Amyloid Precursor Protein

A primary theory for the cause of AD is the overproduction and/or lack of clearance of Aβ peptides derived from APP, especially the more toxic Aβ42 (Mattson, “Pathways Towards and Away from Alzheimer's Disease,” Nature 430:631-639 (2004); Hardy & Selkoe, “The Amyloid Hypothesis of Alzheimer's Disease: Progress and Problems on the Road to Therapeutics,” Science 297(5580):353-6 (2002), which are hereby incorporated by reference in their entirety). Phosphorylation of APP on the Thr668-Pro motif has been shown to be elevated in AD brains and to increase Aβ secretion in vitro (Lee et al., “APP Processing Is Regulated by Cytoplasmic Phosphorylation,” J Cell Biol 163(1):83-95 (2003), which is hereby incorporated by reference in its entirety), although its in vivo significance in regulating APP processing and Aβ production was unknown. Following Thr668 phosphorylation, a new population of the pThr668-Pro motif appears in the cis conformation and exchanges very slowly with the trans form (Ramelot & Nicholson, “Phosphorylation-induced Structural Changes in the Amyloid Precursor Protein Cytoplasmic Tail Detected by NMR,” J Mol Biol 307(3):871-84 (2001); Ramelot et al., “Transient Structure of the Amyloid Precursor Protein Cytoplasmic Tail Indicates Preordering of Structure for Binding to Cytosolic Factors,” Biochemistry 39(10):2714-25 (2000), which are hereby incorporated by reference in their entirety). Since Thr668 phosphorylation is known to be increased during mitosis (Suzuki et al., “Cell Cycle-dependent Regulation of the Phosphorylation and Metabolism of the Alzheimer Amyloid Precursor Protein,” Embo J 13(5):1114-22 (1994), which is hereby incorporated by reference in its entirety), like many other Pin1 substrates (Yaffe et al., “Sequence-specific and Phosphorylation-dependent Proline Isomerization: A Potential Mitotic Regulatory Mechanism,” Science 278:1957-1960 (1997); Lu, “Pinning Down Cell Signaling, Cancer and Alzheimer's Disease,” TiBS 29:200-209 (2004); Lu et al., “The Prolyl Isomerase Pin1 Restores the Function of Alzheimer-associated Phosphorylated Tau Protein,” Nature 399:784-788 (1999); Zhou et al., “Pin1-dependent Prolyl Isomerization Regulates Dephosphorylation of Cdc25C and Tau Proteins,” Mol Cell 6:873-883 (2000), which are hereby incorporated by reference in their entirety), and Pin1 is important for protecting against tauopathy and neurodegeneration (Lu, “Pinning Down Cell Signaling, Cancer and Alzheimer's Disease,” TiBS 29:200-209 (2004); Lu et al., “The Prolyl Isomerase Pin1 Restores the Function of Alzheimer-associated Phosphorylated Tau Protein,” Nature 399:784-788 (1999); Liou et al., “Role of the Prolyl Isomerase Pin1 in Protecting Against Age-dependent Neurodegeneration,” Nature 424:556-561 (2003), which are hereby incorporated by reference in their entirety), it was hypothesized that Pin1 might act on the pThr668-Pro motif to regulate APP processing and Aβ production.

To test this hypothesis, whether Pin1 interacts with APP was examined by transfecting N18 cells with an HA-APP construct, followed by arresting them at mitosis or G1/S to manipulate Thr668 phosphorylation of APP before GST pulldown and co-immunoprecipitation (Yaffe et al., “Sequence-specific and Phosphorylation-dependent Proline Isomerization: A Potential Mitotic Regulatory Mechanism,” Science 278:1957-1960 (1997); Lu et al., “The Prolyl Isomerase Pin1 Restores the Function of Alzheimer-associated Phosphorylated Tau Protein,” Nature 399:784-788 (1999); Lu et al., “A Function of WW Domains as Phosphoserine- or Phosphothreonine-binding Modules,” Science 283:1325-1328 (1999), which are hereby incorporated by reference in their entirety). Pin1 bound to expressed and endogenous APP from mitotic cells (FIG. 4A), and, to a lesser extent, from asynchronous or G1/S cells (FIG. 4B). Similarly, anti-HA monoclonal antibody (mAb) co-immunoprecipitated endogenous Pin1 from mitotic cells and to a lesser degree, from asynchronous or G1/S cells (FIG. 4C). These differences in Pin1 binding to APP correlated with cell cycle-regulated Thr668 phosphorylation (FIGS. 4A-C) (Suzuki et al., “Cell Cycle-dependent Regulation of the Phosphorylation and Metabolism of the Alzheimer Amyloid Precursor Protein,” Embo J 13(5): 1114-22 (1994), which is hereby incorporated by reference in its entirety).

### Example 8

### APP Phosphorylation and Pint Binding

To examine the importance of APP phosphorylation for Pin1 binding, ³⁵S-APP was synthesized and phosphorylated by mitotic extracts, followed by dephosphorylation before GST pulldown (Yaffe et al., “Sequence-specific and Phosphorylation-dependent Proline Isomerization: A Potential Mitotic Regulatory Mechanism,” Science 278:1957-1960 (1997); Lu et al., “The Prolyl Isomerase Pin1 Restores the Function of Alzheimer-associated Phosphorylated Tau Protein,” Nature 399:784-788 (1999), which are hereby incorporated by reference in their entirety). Pin1 bound to phosphorylated APP and the binding was abolished by dephosphorylation and mediated by Pin1 WW domain (FIG. 4D), a known pSer/Thr-Pro-binding module (Lu et al., “A Function of WW Domains as Phosphoserine- or Phosphothreonine-binding Modules,” Science 283:1325-1328 (1999), which is hereby incorporated by reference in its entirety).

To identify the Pin1-binding site in APP, alanine was substituted for Thr668, the only Thr/Ser-Pro motif in the APP intracellular domain (“AICD”). Pin1 failed to bind the APP^{T668A} mutant when expressed in mitotic cells (FIGS. 4A and 4C), indicating that the Thr668-Pro motif is important for binding. To confirm these results, recombinant AICD and AICD^{T668A} were incubated with cyclin B/Cdc2 kinase, followed by dephosphorylation with CIP before GST pulldown. AICD, but not AICD^{T668A}, was phosphorylated by Cdc2 (Suzuki et al., “Cell Cycle-dependent Regulation of the Phosphorylation and Metabolism of the Alzheimer Amyloid Precursor Protein,” Embo J 13(5): 1114-22 (1994), which is hereby incorporated by reference in its entirety). Pin1 bound to Cdc2-phosphorylated AICD, but not AICD^{T668A}, and the binding was abolished by dephosphorylation (FIG. 4E). This binding was mediated by the Pin1 WW domain (FIG. 4F) and effectively competed by a pThr668-containing APP peptide (FIG. 4G). These results together indicate that Pin1 binds to the pThr668-Pro motif in APP in vitro and in vivo and that the binding is mediated by the WW domain, as is in other Pin1 substrates (Lu et al., “Proline-directed Phosphorylation and Isomerization in Mitotic Regulation and in Alzheimer's Disease,” BioEssays 25:174-181 (2003); Lu, “Pinning Down Cell Signaling, Cancer and Alzheimer's Disease,” TiBS 29:200-209 (2004); Lu et al., “A Function of WW Domains as Phosphoserine- or Phosphothreonine-binding Modules,” Science 283:1325-1328 (1999), which are hereby incorporated by reference in their entirety).

### Example 9

### NMR Spectroscopy

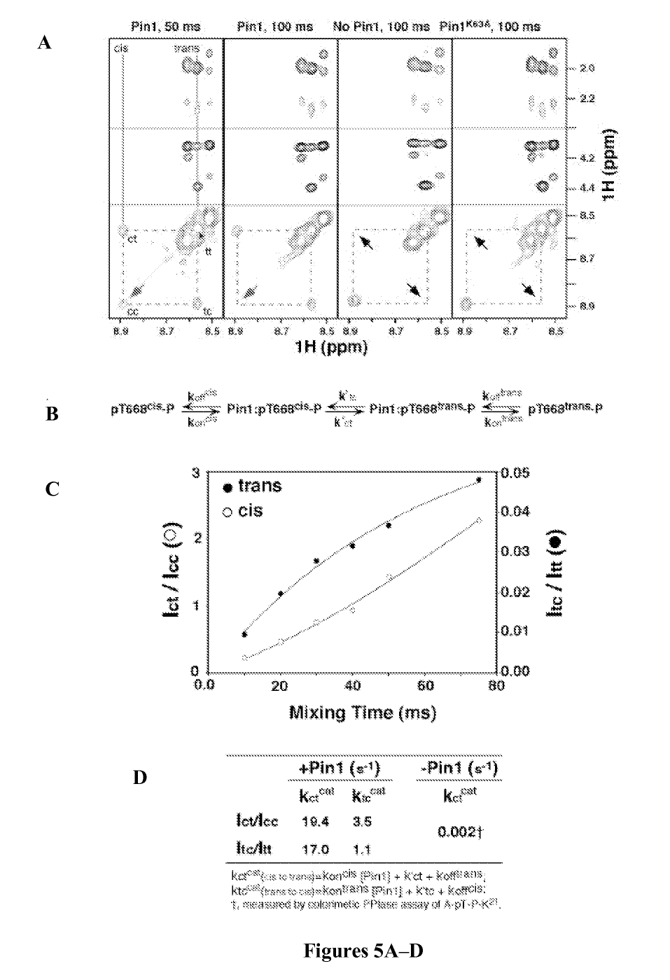

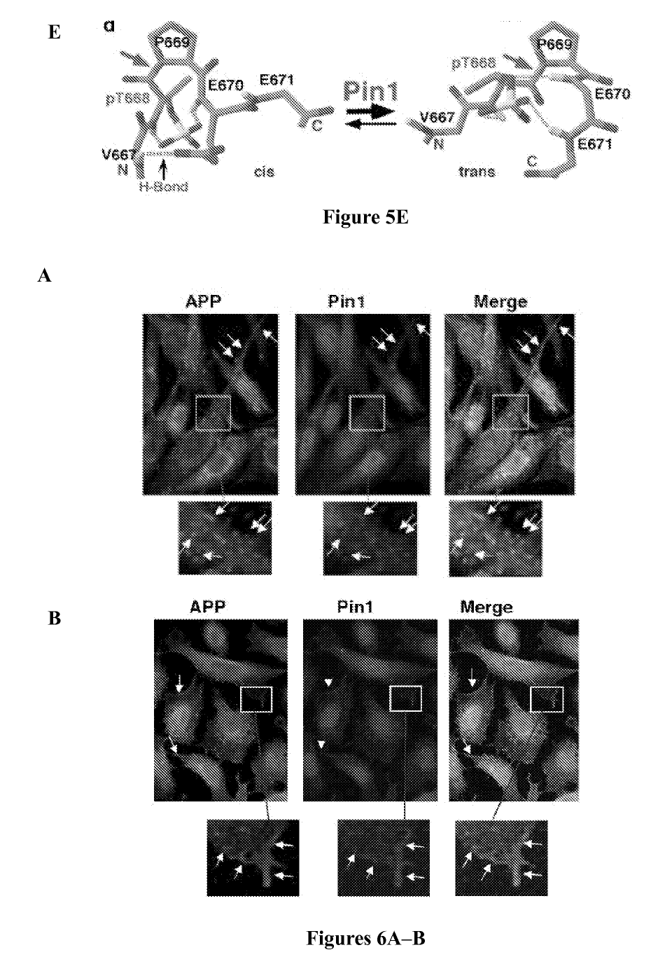

Pin1 is shown to catalyze cis/trans isomerization of pSer/Thr-Pro motifs using biochemical assays (Yaffe et al., “Sequence-specific and Phosphorylation-dependent Proline Isomerization: A Potential Mitotic Regulatory Mechanism,” Science 278:1957-1960 (1997); Zhou et al., “Pin1-dependent Prolyl Isomerization Regulates Dephosphorylation of Cdc25C and Tau Proteins,” Mol Cell 6:873-883 (2000); Stukenberg & Kirschner, “Pin1 Acts Catalytically to Promote a Conformational Change in Cdc25,” Mol Cell 7(5):1071-83 (2001), which are hereby incorporated by reference in their entirety), but such activity has never been visualized with atomic resolution. NMR spectroscopy was used to examine Pin1-catalyzed isomerization of the pThr668-Pro bond in a 21-residue phosphopeptide (G659-Q679 of APP), because this peptide accurately reflects the structural and dynamic features of the corresponding residues in the native AICD (Ramelot & Nicholson, “Phosphorylation-induced Structural Changes in the Amyloid Precursor Protein Cytoplasmic Tail Detected by NMR,” J Mol Biol 307(3):871-84 (2001), which is hereby incorporated by reference in its entirety). Slow exchange between pThr668-Pro isomers yields two distinct sets of ¹H peaks for many residues in the peptide, with the E670 amide proton (E670-H^{N}) particularly well-resolved (FIG. 5A). In the ROESY spectrum, the intensities of exchange and diagonal peaks for a two-state isomerization reaction explicitly depend on the forward (“k_{ct}^{cat}”) and backward (“k_{tc}^{cat}”) rate constants (FIG. 5B), and on the mixing time (“tₘ”). With increasing tₘ, diagonal peaks diminished and exchange peaks grew (FIG. 5A, grey arrows). The same exchange cross peaks were observed upon the addition of a catalytic amount of GST-Pin1 or Pin1; however, exchange cross peaks were absent when either no Pin1 or a catalytically inactive Pin1^{K63A} mutant was added (FIG. 5A, black arrows). Ratios of cross/diagonal peak intensities for cis and trans E670-H^{N} were calculated (FIG. 5C), as described in RICHARD R. ERNST ET AL., PRINCIPLES OF NUCLEAR MAGNETIC RESONANCE IN ONE AND TWO DIMENSIONS (1987), which is hereby incorporated by reference in its entirety, and curve-fits of these ratios vs tₘ provided independent and consistent measures of k_{ct}^{cat} and k_{tc}^{cat} (FIGS. 5C-D). Pin1 accelerated the pThr668-Pro isomerization rate by several orders of magnitude over the typical uncatalyzed isomerization rates for pThr-Pro peptides (Schutkowski et al., “Role of Phosphorylation in Determining the Backbone Dynamics of the Serine/Threonine-Proline Motif and Pin1 Substrate Recognition,” Biochemistry 37(16):5566-75 (1998), which is hereby incorporated by reference in its entirety) and dramatically reduced the average lifetime of the cis (˜0.05 s) and trans (˜0.5 s) isomeric states (FIGS. 5D-E). The k_{ct}^{cat} and k_{tc}^{cat} rates differ by 10-fold, as expected based on the equilibrium populations of free cis and trans. These results provide the first direct atomic level demonstration of Pin1-catalyzed conformational regulation of its substrates.

### Example 10

### Pin1 and APP Processing in Vitro

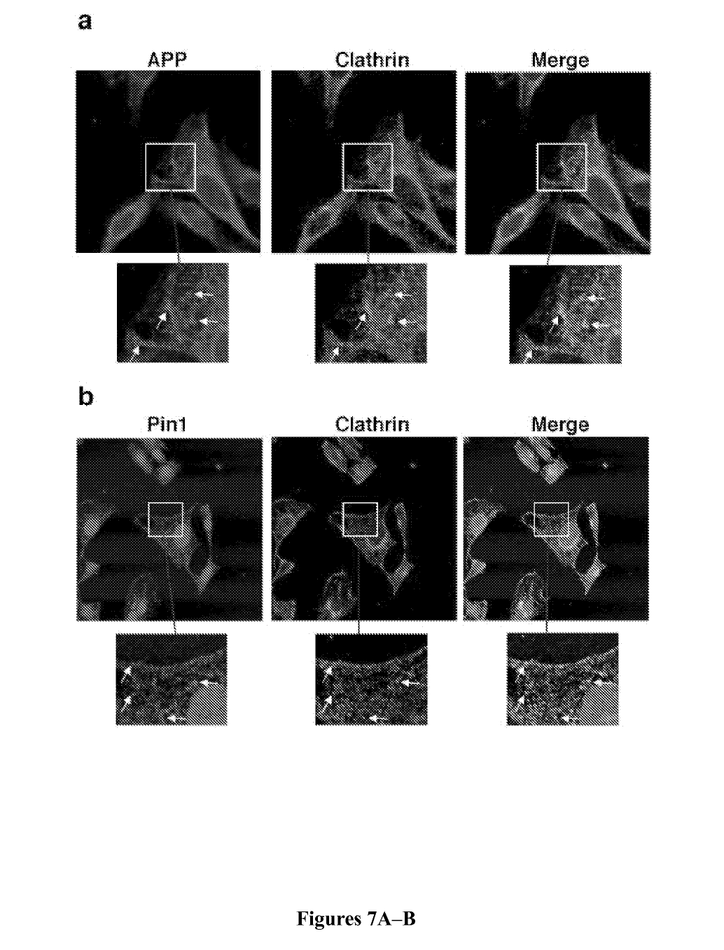

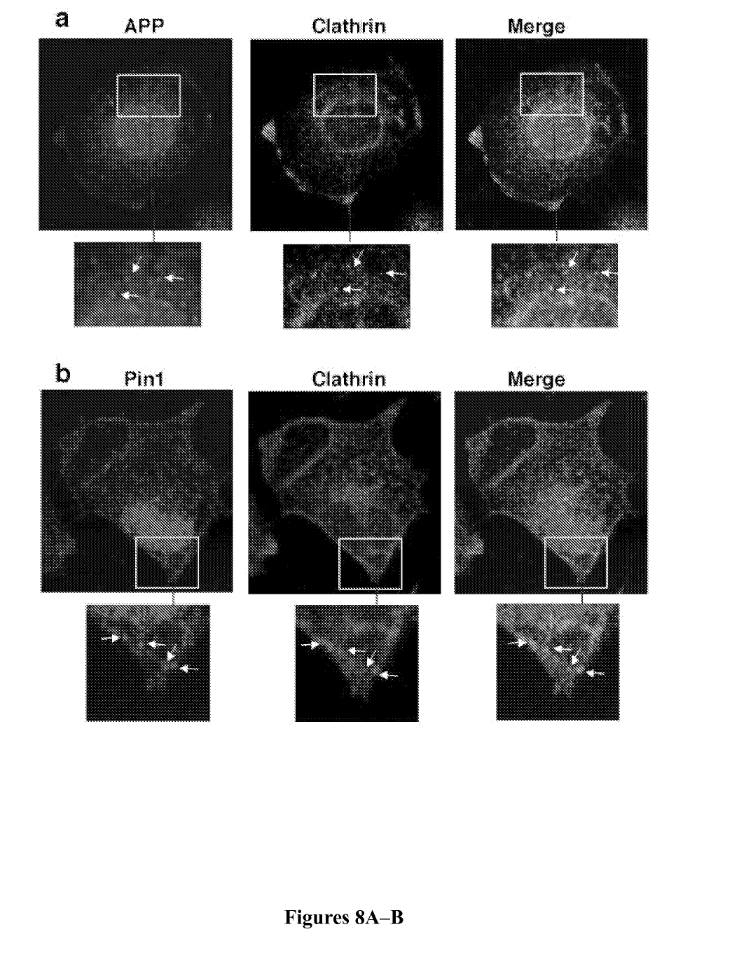

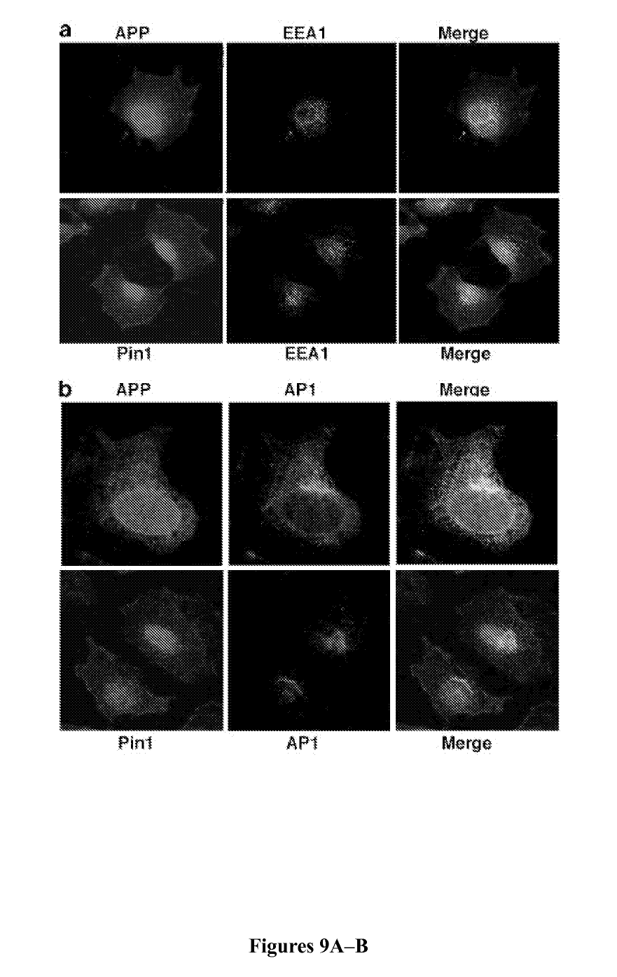

Given that Pin1 binds to the pThr668-Pro motif in APP and greatly accelerates its isomerization, a critical question is whether Pin1 affects APP processing and Aβ production. APP is processed by non-amyloidogenic α-secretases mainly at the plasma membrane and by amyloidogenic β- and γ-secretases at endosomes and other subsequent structures (Mattson, “Pathways Towards and Away from Alzheimer's Disease,” Nature 430:631-639 (2004); Hardy & Selkoe, “The Amyloid Hypothesis of Alzheimer's Disease: Progress and Problems on the Road to Therapeutics,” Science 297(5580):353-6 (2002), which are hereby incorporated by reference in their entirety). Therefore, it is of great importance to examine whether and where Pin1 and APP co-localize in the cell. Pin1 and APP were found to co-localize prominently at the plasma membrane and in intracellular vesicles close to the plasma membrane both in CHO-APP (FIG. 6A) and H4 cells (FIG. 6B). These vesicles were further identified to be clathrin-coated vesicles, but not endosomes or subsequent structures (see FIGS. 7A-B, 8A-B, and 9A-B). Therefore, Pin1 colocalizes with APP primarily at the plasma membrane and clathrin-coated vesicles, suggesting that Pin1 might promote non-amyloidogenic APP processing and reduce Aβ production.

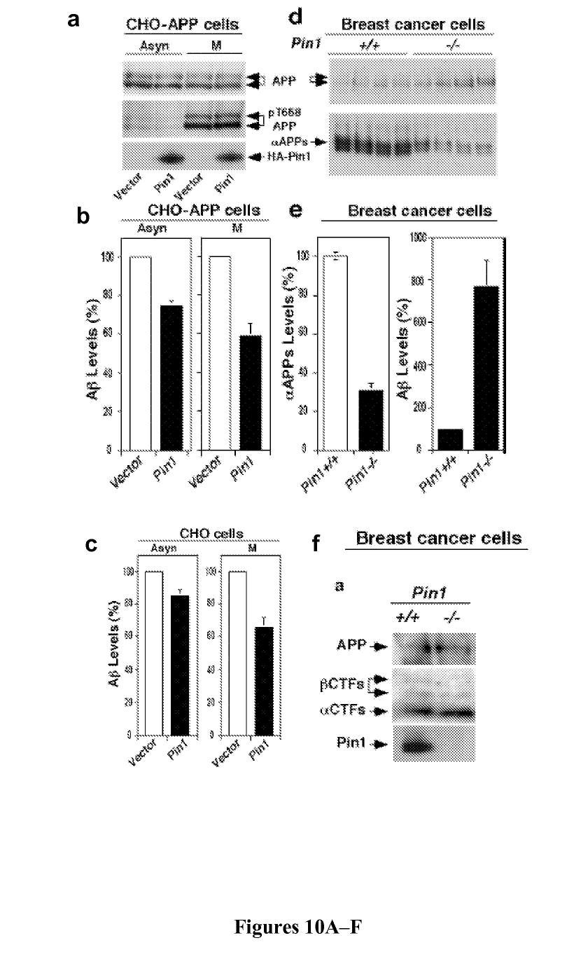

To examine this possibility, first the effects of Pin1 overexpression on Aβ secretion was determined in cultured cells by transfecting CHO-APP cells with a Pin1 construct or CHO cells with Pin1 and APP751 constructs, followed by measuring total Aβ secretion from asynchronous or mitotic cells. Pin1 overexpression in CHO-APP cells (FIGS. 10A-B) and CHO cells (FIG. 10C) significantly reduced Aβ secretion, especially from mitotic cells where Thr668 phosphorylation was elevated. These results show that overexpression of Pin1 reduces Aβ secretion and the effects might depend on Thr668 phosphorylation.

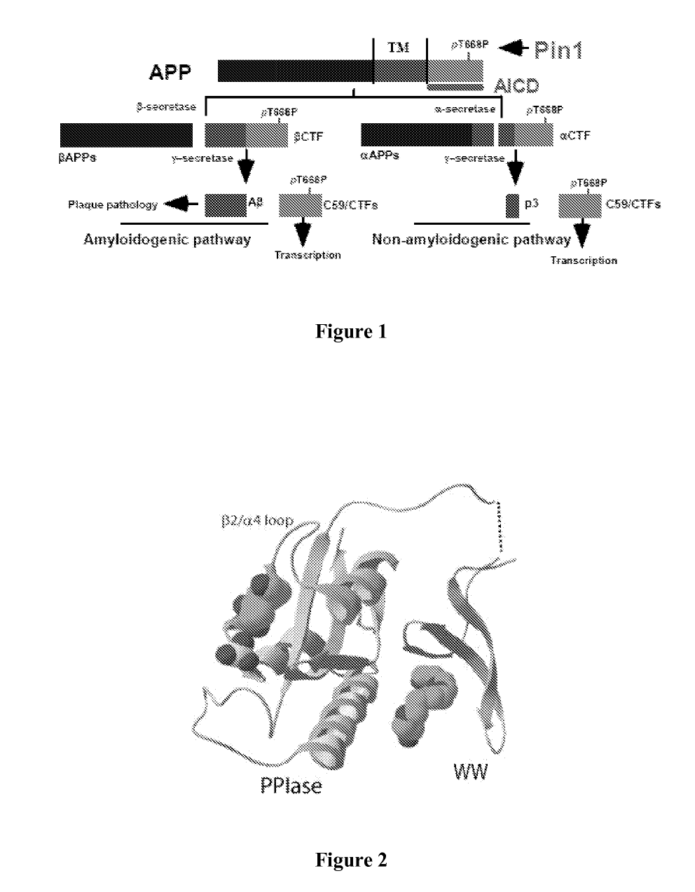

Next the effects of Pin1 knockout (“KO”) on APP processing and Aβ secretion was examined using breast cancer cell lines derived from MMTV-Neu transgenic mice in Pin1+/+ or Pin −/− background (Wulf et al., “Modeling Breast Cancer in Vivo and ex Vivo Reveals an Essential Role of Pin1 in Tumorigenesis,” EMBO J. 23:3397-3407 (2004), which is hereby incorporated by reference in its entirety). As illustrated in FIG. 1, APP is cleaved by α- or β-secretases to generate soluble NH₂-terminal fragments (αAPPs or βAPPs) and membrane-anchored COOH-terminal fragments (αCTFs or βCTFs), respectively (Mattson, “Pathways Towards and Away from Alzheimer's Disease,” Nature 430:631-639 (2004); Hardy & Selkoe, “The Amyloid Hypothesis of Alzheimer's Disease: Progress and Problems on the Road to Therapeutics,” Science 297(5580):353-6 (2002); Pastorino et al., “BACE (β-Secretase) Modulates the Processing of APLP2 in Vivo,” Mol Cell Neurosci 25:642-649 (2004), which are hereby incorporated by reference in their entirety). As shown in FIGS. 10D-F, Pin1+/+ and Pin −/− cancer cell lines expressed comparable levels of endogenous mouse APP, αCTFs and βCTFs (FIG. 10F) or human APP after transfection (FIG. 10D). However, as compared with Pin1+/+ cells, Pin1−/− cells secreted 3 fold less αAPPs (FIGS. 10D-E), as detected by antibody 6E10 specific for human αAPPs, but 7 fold more Aβ (FIG. 10E). These results indicate that Pin1 KO in cells decreases αAPPs, but increases Aβ secretion.

### Example 11

### Pin1 and Aβ Production in Vivo

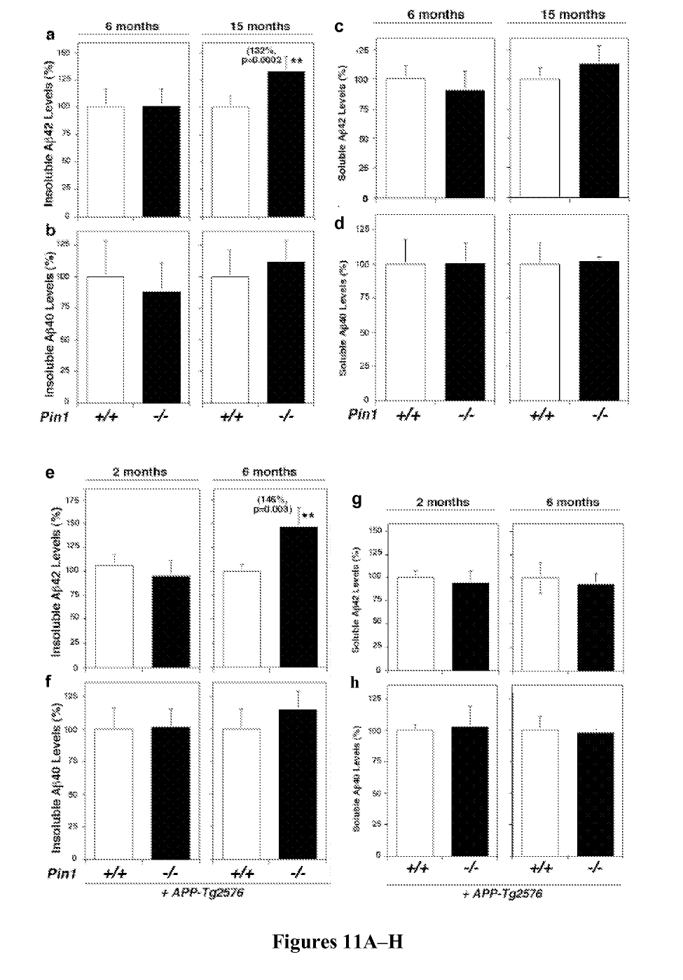

To examine whether Pin1 KO affects APP processing and Aβ production in mice, Aβ production was first examined in Pin1−/− brains using ELISA (Citron et al., “Mutant Presenilins of Alzheimer's Disease Increase Production of 42-Residue Amyloid β-Protein in Both Transfected Cells and Transgenic Mice,” Nat Med 3:67-72 (1997), which is hereby incorporated by reference in its entirety). Sequential proteolysis of APP by β- and γ-secretases generates mainly 40- and 42-residue Aβ peptides (“Aβ40” and “Aβ42”), with Aβ42 being the major toxic species and key contributor to plaque formation in AD (Mattson, “Pathways Towards and Away from Alzheimer's Disease,” Nature 430:631-639 (2004); Hardy & Selkoe, “The Amyloid Hypothesis of Alzheimer's Disease: Progress and Problems on the Road to Therapeutics,” Science 297(5580):353-6 (2002); Citron et al., “Mutant Presenilins of Alzheimer's Disease Increase Production of 42-Residue Amyloid β-Protein in Both Transfected Cells and Transgenic Mice,” Nat Med 3:67-72 (1997); Scheuner et al., “Secreted Amyloid β-Protein Similar to That in the Senile Plaques of Alzheimer's Disease Is Increased in Vivo by the Presenilin 1 and 2 and APP Mutations Linked to Familial Alzheimer's Disease,” Nat Med 2(8):864-70 (1996); Hsiao et al., “Correlative Memory Deficits, Aβ Elevation, and Amyloid Plaques in Transgenic Mice,” Science 274(5284):99-102 (1996); Duff et al., “Increased Amyloid-042(43) in Brains of Mice Expressing Mutant Presenilin 1,” Nature 383(6602):710-3 (1996); Borchelt et al., “Familial Alzheimer's Disease-linked Presenilin 1 Variants Elevate Aβ1-42/1-40 Ratio in Vitro and in Vivo,” Neuron 17(5): 1005-13 (1996), which are hereby incorporated by reference in their entirety). Since the effects of Pin1 KO on neurons in mice are age-dependent (Liou et al., “Role of the Prolyl Isomerase Pin1 in Protecting Against Age-dependent Neurodegeneration,” Nature 424:556-561 (2003), which is hereby incorporated by reference in its entirety), Aβ levels were compared at 2 to 6 months old, when there are no detectable neuronal phenotypes, and 15 months old, when tau hyperphosphorylation and early neurodegeneration are observed (Liou et al., “Role of the Prolyl Isomerase Pin1 in Protecting Against Age-dependent Neurodegeneration,” Nature 424:556-561 (2003), which is hereby incorporated by reference in its entirety). As shown in FIGS. 11A-D, Pin1 KO did not significantly change the levels of Aβ40 or Aβ42 at 2 to 6 months old (FIGS. 11A-B (insoluble levels) and FIGS. 11C-D (soluble levels)), and did not significantly change the levels of soluble Aβ42 (FIG. 11C), soluble Aβ40 (FIG. 11D), or insoluble Aβ40 (FIG. 11B) at 15 months old. However, levels of insoluble Aβ42 were increased by 32% in Pin1−/− brains at 15 months old over Pin1+/+ littermates (FIG. 11A).

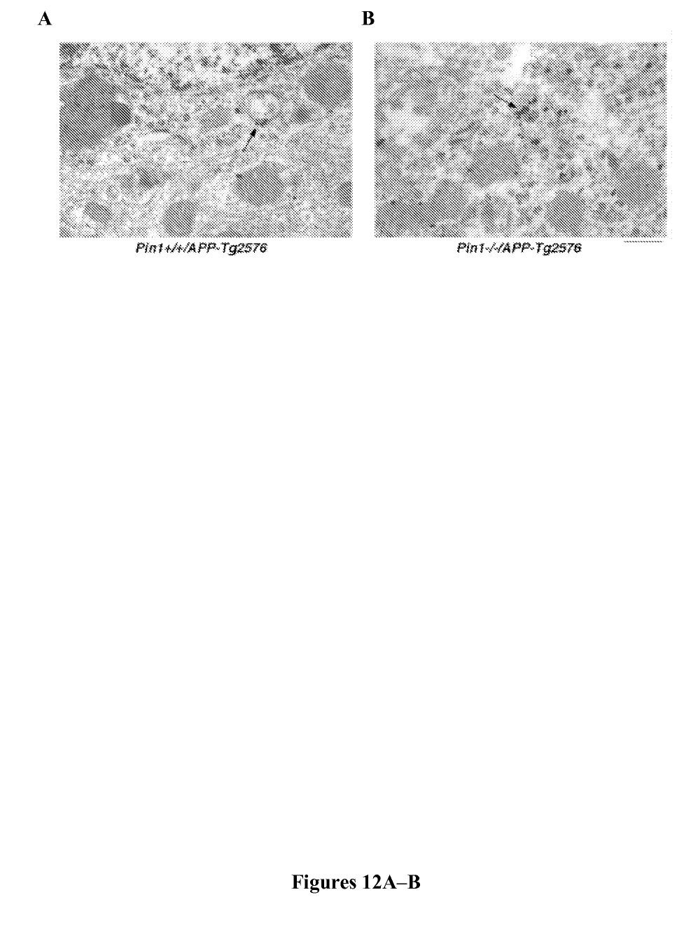

To insure that the Aβ42 increase was not due to early neurodegeneration in Pin1−/− mice and to examine the effects of Pin1 KO on APP processing in vivo, Pin1−/− mice were crossed with APP-Tg25 76 mice overexpressing the FAD APP^{KM670/671NL} mutant (Hsiao et al., “Correlative Memory Deficits, Aβ Elevation, and Amyloid Plaques in Transgenic Mice,” Science 274(5284):99-102 (1996), which is hereby incorporated by reference in its entirety). As shown in FIGS. 11E-H, Pin1 KO did not obviously affect Aβ levels at 2 months old (FIGS. 1G-H), but significantly increased insoluble Aβ, especially Aβ42, by 46% over Pin1+/+ littermates at 6 months old (FIGS. 11E-F) without affecting soluble Aβ40 and Aβ42 (FIGS. 11G-H) or causing neurodegeneration. Immunogold-EM studies showed that Aβ42 was primarily accumulated in multivesicular bodies (“MVB”) of neurons from APP-Tg2576 littermates with or without Pin1 at 7 months old and, notably, that more gold particles were observed in the absence than in the presence of Pin1 (FIGS. 12A-B (1-5 vs. >8 particles per MVB)). These results demonstrate that the increased Aβ42 is prominently localized to MVB, where Aβ42 is known to be in human AD brains and APP-Tg2576 mouse brains before β-amyloid plaque pathology (Takahashi et al., “Intraneuronal Alzheimer Aβ42 Accumulates in Multivesicular Bodies and Is Associated with Synaptic Pathology,” Am J Pathol 161(5): 1869-79 (2002), which is hereby incorporated by reference in its entirety). These results indicate that Pin1 KO in mice causes an age-dependent and selective increase in insoluble Aβ42, which is accelerated by APP overexpression. Of note, similar increases in Aβ42 levels have been documented in transgenic mice overexpressing FAD presenilin mutants (Citron et al., “Mutant Presenilins of Alzheimer's Disease Increase Production of 42-Residue Amyloid β-Protein in Both Transfected Cells and Transgenic Mice,” Nat Med 3:67-72 (1997); Duff et al., “Increased Amyloid-β42(43) in Brains of Mice Expressing Mutant Presenilin 1,” Nature 383(6602):710-3 (1996); Borchelt et al., “Familial Alzheimer's Disease-linked Presenilin 1 Variants Elevate Aβ1-42/1-40 Ratio in Vitro and in Vivo,” Neuron 17(5): 1005-13 (1996), which are hereby incorporated by reference in their entirety), or in FAD brains (Scheuner et al., “Secreted Amyloid β-Protein Similar to That in the Senile Plaques of Alzheimer's Disease Is Increased in Vivo by the Presenilin 1 and 2 and APP Mutations Linked to Familial Alzheimer's Disease,” Nat Med 2(8):864-70 (1996), which is hereby incorporated by reference in its entirety), indicating that small shifts in Aβ42 production may have an important impact on the development of AD.

### Example 12

### Pin1 and APP Processing in Vivo

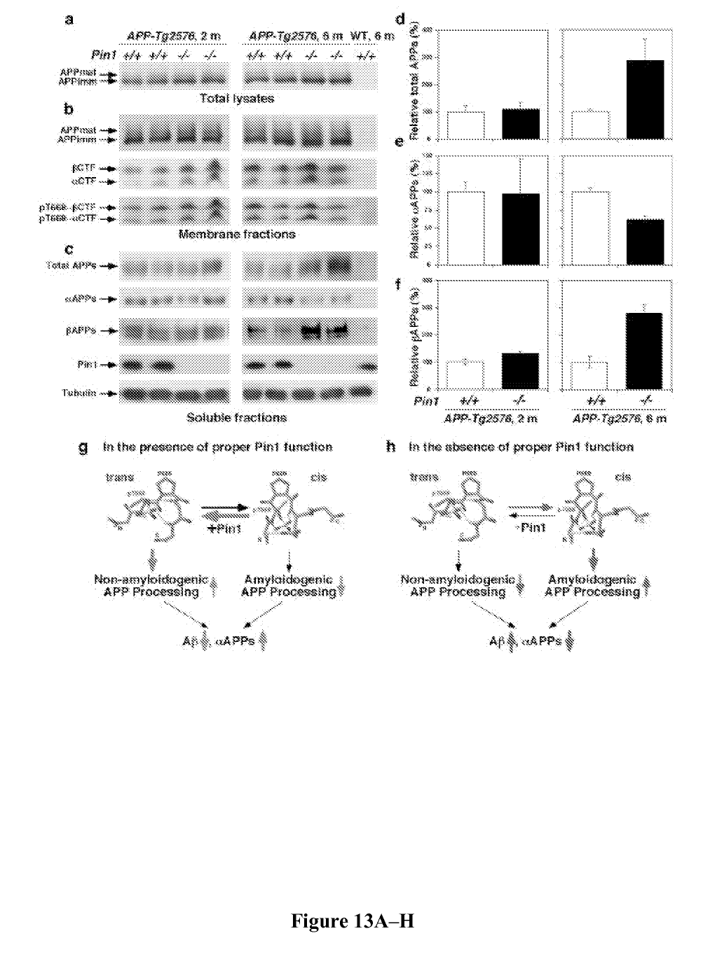

The results described in Example 11 indicate that Pin1 KO increases Aβ42 production, suggesting that it might favor amyloidogenic APP processing. To investigate this possibility, changes in APP processing were examined in APP-Tg2576 mice in the presence or absence of Pin1 at 2 and 6 months old (Pastorino et al., “BACE (03-Secretase) Modulates the Processing of APLP2 in Vivo,” Mol Cell Neurosci 25:642-649 (2004), which is hereby incorporated by reference in its entirety). Pin1 KO had no obvious effects on total APP, CTFs, or their Thr668 phosphorylation, but significantly affected total APPs, αAPPs and βAPPs at 6 months old, but not at 2 months old, of APP-Tg2576 mice (FIGS. 13A-F). Total APPs and βAPPs levels were increased by 3 fold, but αAPPs was reduced by 50% in Pin1−/− brains as compared with Pin1+/+ controls (FIGS. 13A-F). These results indicate that Pin1 KO increases amyloidogenic vs. non-amyloidogenic APP processing and elevates Aβ42 in an age-dependent manner.

These results identify Pin1-catalyzed prolyl isomerization as a novel mechanism to regulate APP processing and Aβ production relevant to AD. Thr668-Pro phosphorylation may act as a conformational switch by increasing the cis content from 0% to 10% (Ramelot & Nicholson, “Phosphorylation-induced Structural Changes in the Amyloid Precursor Protein Cytoplasmic Tail Detected by NMR,” J Mol Biol 307(3):871-84 (2001), which is hereby incorporated by reference in its entirety). In the absence of a catalyst, cis/trans isomerization may present a major rate-limiting step for biological processes because the distinct isomer structures likely interact with different cellular proteins. Given the dramatic effects of Pin1 on the APP conformational dynamics in vitro and on APP processing and Aβ production in cell cultures and animals, it is hypothesized that the cis pThr668-Pro conformation may favor amyloidogenic APP processing, whereas the trans conformation may favor non-amyloidogenic APP processing, and by catalyzing their conversion, Pin1 promotes non-amyloidogenic APP processing and reduces Aβ production (FIG. 13G-H).

Although APP is likely phosphorylated on the Thr668-Pro motif in trans (Lu, “Pinning Down Cell Signaling, Cancer and Alzheimer's Disease,” TiBS 29:200-209 (2004), which is hereby incorporated by reference in its entirety), this motif partitions into both trans and cis isomers due to local structural constraints after phosphorylation (Ramelot & Nicholson, “Phosphorylation-induced Structural Changes in the Amyloid Precursor Protein Cytoplasmic Tail Detected by NMR,” J Mol Biol 307(3):871-84 (2001); Ramelot et al., “Transient Structure of the Amyloid Precursor Protein Cytoplasmic Tail Indicates Preordering of Structure for Binding to Cytosolic Factors,” Biochemistry 39(10):2714-25 (2000), which are hereby incorporated by reference in their entirety). Pin1 would rapidly reestablish equilibrium if the trans (or cis) population were suddenly depleted. In the non-equilibrium cellular environment (FIG. 13G), Pin1-catalyzed prolyl isomerization might prevent an increase in amyloidogenic APP processing and Aβ production by dynamically linking cis and trans populations and preventing the build-up of cis. However, if Pin1 function is absent as in Pin1−/− mice or cells, or downregulated/inhibited by oxidation or genetic alterations as in AD (FIG. 13H) (Lu et al., “The Prolyl Isomerase Pin1 Restores the Function of Alzheimer-associated Phosphorylated Tau Protein,” Nature 399:784-788 (1999); Zhou et al., “Pin1-dependent Prolyl Isomerization Regulates Dephosphorylation of Cdc25C and Tau Proteins,” Mol Cell 6:873-883 (2000); Liou et al., “Role of the Prolyl Isomerase Pin1 in Protecting Against Age-dependent Neurodegeneration,” Nature 424:556-561 (2003); Sultana et al., “Oxidative Modification and Down-regulation of Pin1 in Alzheimer's Disease Hippocampus: A Redox Proteomics Analysis,” Neurobiol Aging 27(7):918-25 (2006 (Epub 2005)); Segat et al., “Pin1 Promoter Polymorphisms are Associated with Alzheimer's Disease,” Neurobiol Aging 28(1):69-74 (2007 (Epub 2005)), which are hereby incorporated by reference in their entirety), a higher concentration of cis pThr668-Pro motif would be present for a longer time due to the extremely slow uncatalyzed isomerization rate, which might promote amyloidogenic APP processing. It has been shown that Pin1 binds to and isomerizes the pThr231-Pro motif in tau (Lu et al., “The Prolyl Isomerase Pin1 Restores the Function of Alzheimer-associated Phosphorylated Tau Protein,” Nature 399:784-788 (1999); Zhou et al., “Pin1-dependent Prolyl Isomerization Regulates Dephosphorylation of Cdc25C and Tau Proteins,” Mol Cell 6:873-883 (2000), which are hereby incorporated by reference in their entirety) and its KO causes an age-dependent accumulation of the pThr231-Pro motif in the AD tangle-specific conformation that is likely also in cis (Liou et al., “Role of the Prolyl Isomerase Pin1 in Protecting Against Age-dependent Neurodegeneration,” Nature 424:556-561 (2003), which is hereby incorporated by reference in its entirety). Therefore, Pin1 deregulation might have similar conformational effects on specific pThr-Pro motifs in APP and tau, although they might lead to different pathological changes with the similar neurodegenerative outcome (Lu et al., “The Prolyl Isomerase Pin1 Restores the Function of Alzheimer-associated Phosphorylated Tau Protein,” Nature 399:784-788 (1999); Zhou et al., “Pin1-dependent Prolyl Isomerization Regulates Dephosphorylation of Cdc25C and Tau Proteins,” Mol Cell 6:873-883 (2000); Liou et al., “Role of the Prolyl Isomerase Pin1 in Protecting Against Age-dependent Neurodegeneration,” Nature 424:556-561 (2003), which are hereby incorporated by reference in their entirety).

### Example 13

### Cis-Enriched Phosphopeptide Mimetics

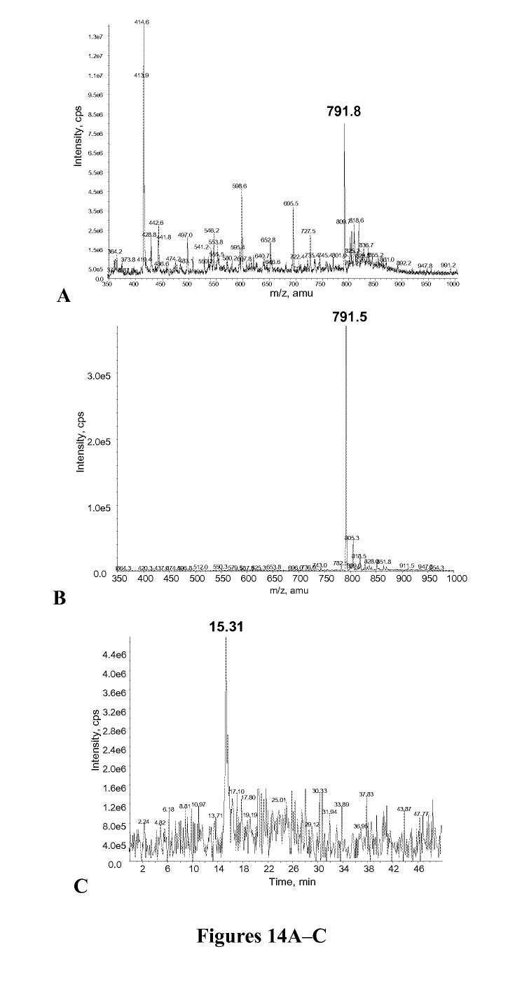

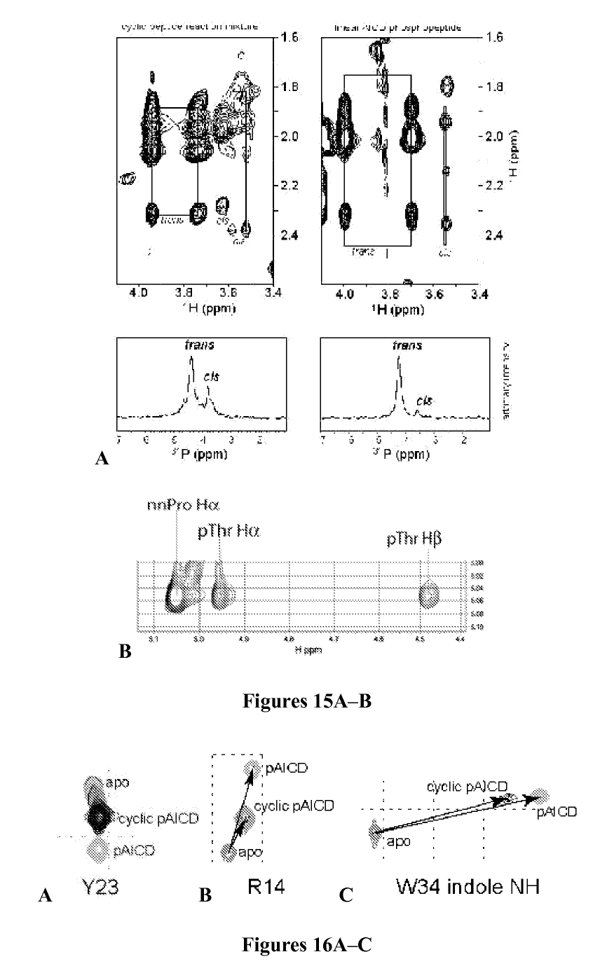

The cis conformation of the pThr-Pro motif in AICD is unique from the trans isomer in that it is stabilized by different hydrogen bonds, and it is more extended in backbone conformation, aside from the kink of the cis peptide bond (Ramelot & Nicholson, “Phosphorylation-induced Structural Changes in the Amyloid Precursor Protein Cytoplasmic Tail Detected by NMR,” J Mol Biol 307(3):871-84 (2001), which is hereby incorporated by reference in its entirety). It was hypothesized that the cis conformation could be stabilized by formation of a covalent bond between V667 NH and E670 COO— (hydrogen bonded in the NMR structure), thereby stabilizing the cis isomer relative to the trans. A 6-residue phosphopeptide corresponding to APP residues V667-R72 ([NH₂]-V-pT-P-E-E-R-resin (SEQ ID NO: 1)) was commercially synthesized with an orthogonal blocking group on E4 (Tufts Core Facility), and sent on the resin. E4 was selectively deprotected in 1M TBAF in DMF for 1 hour, the resin was washed with DMF, then washed into cyclization solution (DCC and HOBT in DMF). Because this sequence adopts only 10% cis and 90% trans conformation and interconversion is slow, the resin-immobilized phosphopeptide was cycled between cyclization reaction conditions and Pin1-catalyzed isomerization conditions (10 mM HEPES, 10 mM NaCl, 1 mM DTT, pH 7, 50 uM Pin1) over a period of 3 days and monitored using the ninhydrin test. Cleavage of the remaining side chain blocking groups and phosphopeptide from the resin was accomplished by addition of trifluoroacetic acid (“TFA”) followed by passage through a scintered glass filter to remove the resin, and evaporation to a minimal amount of TFA under a stream of N₂ gas in the hood. Phosphopeptide products were separated from side chain blocking groups by water/ether extraction and the water phase was subsequently lyophilized to dryness. On-line LC/MS/MS using a triple quadrupole linear ion trap (4000 Q Trap) performed in Cornell's BioResource Center showed that the water/ether extraction was incomplete (FIG. 14A), but that the cyclization was around ˜99% complete (FIG. 14B). Moreover, the retention time of the cyclized product on the reversed phase capillary C18 column indicates that HPLC purification would be straightforward (FIG. 14C). Since the reaction mixture contains effectively “100%” cyclized phosphopeptide (vs. linear), NMR was used directly on the reaction mixture to determine cis and trans populations. 2D ¹H—¹H TOCSY and 1D 31p spectra yielded the same cis (˜30%) and trans (70%) populations, demonstrating that this peptide increases the cis isomer equilibrium population (FIGS. 15A-B). The spectra show two cis populations that most likely reflect different hydrogen bond geometries adopted in the cis isomer (Ramelot & Nicholson, “Phosphorylation-induced Structural Changes in the Amyloid Precursor Protein Cytoplasmic Tail Detected by NMR,” J Mol Biol 307(3):871-84 (2001), which is hereby incorporated by reference in its entirety).

This cyclic pAICD (30% cis, 70% trans) has been found to interact with the WW domain of Pin1 in a manner consistent with an increase in the cis population, as shown in FIGS. 16A-C. Natural abundance peptide (either the linear or cyclic pAICD peptide) was incrementally added to {¹⁵N}-WW and peak shifts were monitored via ¹⁵N—¹H HSQC spectra. Saturation with cyclic pAICD induces smaller peak shifts than the linear pAICD.

### Example 14

### Possible Synthetic Route for Formula II Compounds

A possible synthetic route for a compound of formula II based on well-characterized chemical synthesis reactions is shown in Scheme 1. The synthesis begins with L-Proline and crotonoyl chloride, proceeds through epoxidation, and then cyclization under basic conditions. The addition of a phosphate group to the hydroxyl group of the product could be accomplished, for example, as described by Bannwarth & Trzeciak “A Simple and Effective Chemical Phosphorylation Procedure for Biomolecules,” Helvetica Chimica Acta 70:175-86 (1987), which is hereby incorporated by reference in its entirety). If this suggested synthetic route is taken, two new chiral centers are produced, resulting in four possible sterioisomers of the product. One stereoisomer is shown in Scheme 1.

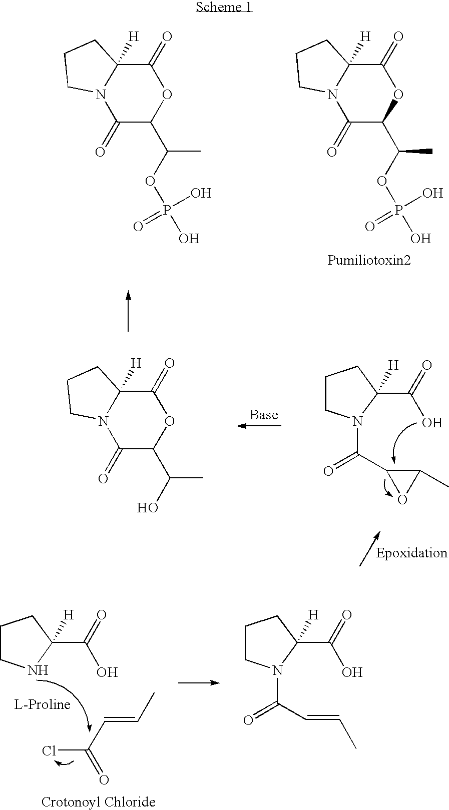

### Example 15

### Identification of Pint Residues Critical for pThr-Pro Binding

A high-resolution structure of Pin1 complexed with an Ala-Pro peptide has been solved, and its enzymatic properties and substrate specificity defined (Ranganathan et al., “Structural and Functional Analysis of the Mitotic Rotamase Pin1 Suggests Substrate Recognition Is Phosphorylation Dependent,” Cell 89:875-886 (1997); Yaffe et al., “Sequence-specific and Phosphorylation-dependent Proline Isomerization: A Potential Mitotic Regulatory Mechanism,” Science 278:1957-1960 (1997), which are hereby incorporated by reference in their entirety). Based on molecular modeling as well as site-directed and random mutagenesis, a number of amino acid residues have been found that might play important roles in affecting Pin1-substrate interactions (such as Arg68 and Arg69) or catalysis (such as His59, Cys113, L122, M130, F134, H157 and S154) (Zhou et al., “Pin1-dependent Prolyl Isomerization Regulates Dephosphorylation of Cdc25C and Tau Proteins,” Mol Cell 6:873-883 (2000), which is hereby incorporated by reference in its entirety). By comparing Pin1 with Pin1-related PPIases, including bacterial parvulin, that almost all of the active site residues of parvulin were found to be identical to those in Pin1, except for Arg-68 and -69, which have been replaced by Glu in parvulin (Rahfeld et al., “A Novel Peptidyl-prolyl Cis/Trans Isomerase from Escherichia coli,” FEBS Lett 343:65-69 (1994), which is hereby incorporated by reference in its entirety). Parvulin fails to catalyze the isomerization of phosphorylated Ser-Pro peptidyl bonds, although it is active in catalyzing the Pro isomerization of the unphosphorylated peptide (Rahfeld et al., “A Novel Peptidyl-prolyl Cis/Trans Isomerase from Escherichia coli,” FEBS Lett 343:65-69 (1994); Uchida et al., “Identification and Characterization of a 14 kDa Human Protein as a Novel Parvulin-like Peptidyl Prolyl Cis/Trans Isomerase,” FEBS Lett 446:278-82 (1999), which are hereby incorporated by reference in their entirety). Therefore, the PPIase activity appears to have evolved before the phosphorylation-specific activity of the molecules, like Pin1. Indeed, substitutions of Arg-68 and -69 with Ala generate a mutant that is PPIase-positive but is unable to catalyze isomerization of pSer/Thr-Pro motifs (Yaffe et al., “Sequence-specific and Phosphorylation-dependent Proline Isomerization: A Potential Mitotic Regulatory Mechanism,” Science 278:1957-1960 (1997), which is hereby incorporated by reference in its entirety). Conversely, those residues conserved from bacteria to humans are likely to be involved in the fundamentals of isomerizing catalysis. Indeed, a substitution of Lys63 with Ala generates a completely PPIase-inactive mutant (Zhou et al., “Pin1-dependent Prolyl Isomerization Regulates Dephosphorylation of Cdc25C and Tau Proteins,” Mol Cell 6:873-883 (2000), which is hereby incorporated by reference in its entirety), which is also confirmed by NMR analysis (Pastorino et al., “The Prolyl Isomerase Pin1 Regulates Amyloid Precursor Protein Processing and Amyloid-β Production,” Nature 440(7083):528-34 (2006), which is hereby incorporated by reference in its entirety). To perform an unbiased analysis, over 50 Pin1 point mutants were generated by PCR-based random mutagenesis, and then examined for their ability to rescue the ts ESS1 phenotype in yeast and to bind and isomerize pSer/Thr-Pro motifs (Zhou et al., “Pin1-dependent Prolyl Isomerization Regulates Dephosphorylation of Cdc25C and Tau Proteins,” Mol Cell 6:873-883 (2000); Lu et al., “A Function of WW Domains as Phosphoserine- or Phosphothreonine-binding Modules,” Science 283:1325-1328 (1999), which are hereby incorporated by reference in their entirety). Approximately 40% of Pin1 mutants failed to function in yeast and they are concentrated on a number of important residues. Point mutations of certain residues that are conserved between Pin1 and bacterial parvulin (L60P and L61P), or are unique to Pin1 (S67E and S71P) disrupt the ability of Pin1 to isomerize pThr-Pro motifs or to rescue yeast lethal phenotypes even under overexpression, indicating the essential role of Pin1 PPIase activity (Zhou et al., “Pin1-dependent Prolyl Isomerization Regulates Dephosphorylation of Cdc25C and Tau Proteins,” Mol Cell 6:873-883 (2000); Lu et al., “A Function of WW Domains as Phosphoserine- or Phosphothreonine-binding Modules,” Science 283:1325-1328 (1999), which are hereby incorporated by reference in their entirety). In addition, point mutations of certain residues in the Pin1 WW domain (W10R and Y23A) have no effect on the PPIase activity, but disrupt the ability of Pin1 to binds phosphoproteins (Lu et al., “A Function of WW Domains as Phosphoserine- or Phosphothreonine-binding Modules,” Science 283:1325-1328 (1999), which is hereby incorporated by reference in its entirety). Like the isolated PPIase domain, these mutants can rescue the yeast lethal phenotype only under overexpression (Lu et al., “A Function of WW Domains as Phosphoserine- or Phosphothreonine-binding Modules,” Science 283:1325-1328 (1999), which is hereby incorporated by reference in its entirety), indicating that the WW domain is essential under normal condition by targeting Pin1 to substrates.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of inhibiting amyloidogenic processing of amyloid precursor protein, said method comprising:
accelerating cis/trans isomerization of the amyloid precursor protein at a phosphorylated serine/threonine-proline motif under conditions effective to inhibit amyloidogenic processing of the amyloid precursor protein.

2. The method according to claim 1, wherein the method is carried out in vivo.

3. The method according to claim 1, wherein the method is carried out in vitro.

4. The method according to claim 1, wherein said accelerating is carried out with one or more isomerization catalysts selected from the group consisting of Pin 1, Pin1 homologues, catalytic antibodies, and RNA aptamers.

5. The method according to claim 1, wherein the phosphorylated serine/threonine-proline motif is a phosphorylated threonine-668-proline motif.

6. A method of inhibiting production of amyloid beta peptides by a cell, said method comprising:
contacting the cell with a compound that mimics the cis conformation of a phosphorylated serine/threonine-proline motif of an amyloid precursor protein under conditions effective to inhibit production of amyloid beta peptides by the cell.

7. The method according to claim 6, wherein the method is carried out in vivo.

8. The method according to claim 6, wherein the method is carried out in vitro.

9. The method according to claim 6, wherein the phosphorylated serine/threonine-proline motif is a phosphorylated threonine-668-proline motif.

10. The method according to claim 6, wherein the compound is a compound of formula 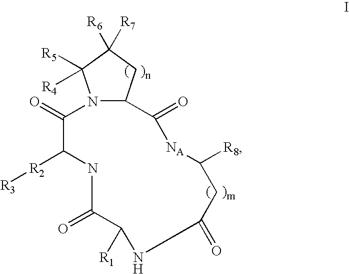
wherein:
R₁ is an amino acid side chain;
R₂ is a glutamic acid-based side chain, an aspartic acid-based side chain, or a moiety of the formula -Ser/Thr-X—Y₍₂₎, where Ser/Thr is a serine amino acid-based side chain or a threonine amino acid-based side chain, X is a negatively charged tetra- or penta-valent moiety selected from the group consisting of —OPO₃²⁻, —PO₃²⁻, —OSO₃²⁻, and —OBO₂²⁻, and Y is independently hydrogen, a blocking group, or absent;
R₃ is absent or a linker between R₂ and N_{A};
R₄ and R₅ are independently hydrogen or C₁₋₃ alkyl;
R₆ and R₇ are independently hydrogen or halogen;
R₈ is —COR where R is a peptide of 0 to approximately 40 amino acid units;
m is 1 or 2;
n is 1, 2, or 3; and
R₁ and/or R₈ are optionally modified to facilitate transport and/or cellular uptake of the compound and/or attachment of the compound to a substrate; and
wherein the compound mimics the cis conformation of a phosphorylated serine/threonine-proline motif of an amyloid precursor protein.

11. The method according to claim 10, wherein the compound is selected from the group consisting of the compounds of formula I set forth in Table 1.

12. The method according to claim 6, wherein the compound is a compound of formula 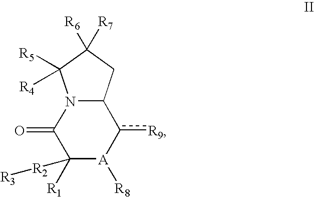
wherein:
R₁ is —H or —NHR₃ where Rₐ is a peptide of 0 to approximately 40 amino acid units;
R₂ is a glutamic acid-based side chain, an aspartic acid-based side chain, or a moiety of the formula -Ser/Thr-X—Y₍₂₎, where Ser/Thr is a serine amino acid-based side chain or a threonine amino acid-based side chain, X is a negatively charged tetra- or penta-valent moiety selected from the group consisting of —OPO₃²⁻, —PO₃²⁻, —OSO₃²⁻, and —OBO₂²⁻, and Y is independently hydrogen, a blocking group, or absent;
R₃ is absent or a linker between R₂ and A;
R₄ and R₅ are independently hydrogen or C₁₋₃ alkyl;
R₆ and R₇ are independently hydrogen or halogen;
R₈ is —H or —CH(CH₂)₂COOHCOR_{b} where R_{b} is a peptide of 0 to approximately 40 amino acid units;
R₉ is a hydrogen bond acceptor;
A is N, O, C, or S; [symbol]
is a single or double bond; and
R₁ and/or R₈ are optionally modified to facilitate transport and/or cellular uptake of the compound and/or attachment of the compound to a substrate; and
wherein the compound mimics the cis conformation of a phosphorylated serine/threonine-proline motif of an amyloid precursor protein.

13. The method according to claim 12, wherein the compound is selected from the group consisting of the compounds of formula II set forth in Table 1.

14. A method of screening for a therapeutic agent effective in treating and/or preventing in a subject a degenerative neurological disease characterized by amyloidogenic processing of amyloid precursor protein and/or overproduction of amyloid beta peptide, said method comprising:
providing a substrate compound comprising a phosphorylated serine/threonine-proline motif of an amyloid precursor protein;
providing a candidate compound;
contacting the candidate compound with the substrate compound;
measuring the cis/trans isomerization rate of the phosphorylated serine/threonine-proline motif in the presence of the candidate compound; and
comparing the cis/trans isomerization rate in the presence of the candidate compound to a reference cis/trans isomerization rate, where acceleration of the cis/trans isomerization rate in the presence of the candidate compound relevant to the reference cis/trans isomerization rate indicates that the candidate compound is a potential therapeutic agent effective in treating and/or preventing in a subject a degenerative neurological disease characterized by amyloidogenic processing of amyloid precursor protein and/or overproduction of amyloid beta peptide.

15. The method according to claim 14, wherein the substrate compound comprises an amyloid precursor protein.

16. The method according to claim 14, wherein the substrate compound is GVVEVDAAVpTPEERHLSKMQQ (SEQ ID NO: 12).

17. The method according to claim 14, wherein the phosphorylated serine/threonine-proline motif is a phosphorylated threonine-668-proline motif.

18. The method according to claim 14, wherein the phosphorylated serine/threonine-proline motif is a derivative of the phosphorylated threonine-668-proline motif.

19. A method of screening for a therapeutic agent effective in treating and/or preventing in a subject a degenerative neurological disease characterized by amyloidogenic processing of amyloid precursor protein and/or overproduction of amyloid beta peptide, said method comprising
providing a temperature sensitive Ess1/Ptf1 mutant yeast cell,
contacting the cell with a candidate compound,
culturing the cell at a temperature effective to cause terminal mitotic arrest of the cell due to an absence of Ess1/Ptf1 function,
evaluating whether the cell displays a temperature-sensitive phenotype during said culturing, and
identifying compounds that prevent the yeast cell from displaying the temperature-sensitive phenotype as likely therapeutic agents effective in treating and/or preventing in a subject a degenerative neurological disease characterized by amyloidogenic processing of amyloid precursor protein and/or overproduction of amyloid beta peptide.

20. The method according to claim 19, wherein the yeast cell is a YPM2 ts cell.

21. The method according to claim 19, wherein the candidate compound is an isomerization catalyst that is based on a transition state analog of a phosphorylated serine/threonine-proline motif of an amyloid precursor protein.

22. A method of screening for biological molecules likely to be involved in the amyloidogenic pathway, said method comprising:
(i) contacting an amyloid precursor protein which is phosphorylated at a serine/threonine-proline motif with a neuronal cell lysate and detecting binding of biological molecules from the neuronal cell lysate to the amyloid precursor protein;
(ii) contacting a compound that mimics the cis conformation of a phosphorylated serine/threonine-proline motif of an amyloid precursor protein with a neuronal cell lysate and detecting binding of biological molecules from the neuronal cell lysate to the compound, under conditions essentially the same as in step (i); and
(iii) comparing the binding detected in step (i) with the binding detected in step (ii), wherein a biological molecule which undergoes greater binding in step (ii) than in step (i) is likely to be involved in the amyloidogenic pathway.

23. The method according to claim 22, wherein said contacting is carried out on an affinity column.

24. The method according to claim 22, wherein the phosphorylated serine/threonine-proline motif is a phosphorylated threonine-668-proline motif.

25. The method according to claim 22, wherein the phosphorylated serine/threonine-proline motif is a derivative of the phosphorylated threonine-668-proline motif.

26. The method according to claim 22, wherein the compound that mimics the cis conformation of a phosphorylated threonine-proline motif of an amyloid precursor protein is a compound of formula 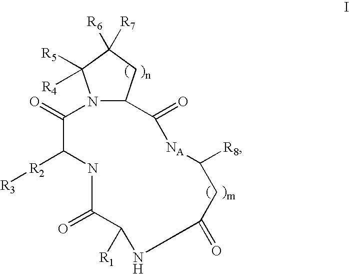
wherein:
R₁ is an amino acid side chain;
R₂ is a glutamic acid-based side chain, an aspartic acid-based side chain, or a moiety of the formula -Ser/Thr-X—Y₍₂₎, where Ser/Thr is a serine amino acid-based side chain or a threonine amino acid-based side chain, X is a negatively charged tetra- or penta-valent moiety selected from the group consisting of —OPO₃²⁻, —PO₃²⁻, —OSO₃²⁻, and —OBO₂²⁻, and Y is independently hydrogen, a blocking group, or absent;
R₃ is absent or a linker between R₂ and N_{A};
R₄ and R₅ are independently hydrogen or C₁₋₃ alkyl;
R₆ and R₇ are independently hydrogen or halogen;
R₈ is —COR where R is a peptide of 0 to approximately 40 amino acid units;
m is 1 or 2;
n is 1, 2, or 3; and
R₁ and/or R₈ are optionally modified to facilitate transport and/or cellular uptake of the compound and/or attachment of the compound to a substrate; and
wherein the compound mimics the cis conformation of a phosphorylated serine/threonine-proline motif of an amyloid precursor protein.

27. The method according to claim 26, wherein the compound is selected from the group consisting of the compounds of formula I set forth in Table 1.

28. The method according to claim 22, wherein the compound that mimics the cis conformation of a phosphorylated threonine-proline motif of an amyloid precursor protein is a compound of formula 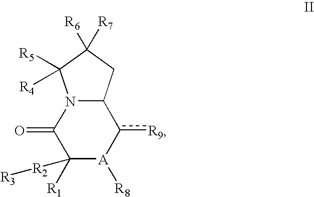
wherein:
R₁ is —H or —NHRₐ where Rₐ is a peptide of 0 to approximately 40 amino acid units;
R₂ is a glutamic acid-based side chain, an aspartic acid-based side chain, or a moiety of the formula -Ser/Thr-X—Y₍₂₎, where Ser/Thr is a serine amino acid-based side chain or a threonine amino acid-based side chain, X is a negatively charged tetra- or penta-valent moiety selected from the group consisting of —OPO₃²⁻, —PO₃²⁻, —OSO₃²⁻, and —OBO₂²⁻, and Y is independently hydrogen, a blocking group, or absent;
R₃ is absent or a linker between R₂ and A;
R₄ and R₅ are independently hydrogen or C₁₋₃ alkyl;
R₆ and R₇ are independently hydrogen or halogen;
R₈ is —H or —CH(CH₂)₂COOHCOR_{b} where R_{b} is a peptide of 0 to approximately 40 amino acid units;
R₉ is a hydrogen bond acceptor;
A is N, O, C, or S; [symbol]
is a single or double bond; and
R₁ and/or R₈ are optionally modified to facilitate transport and/or cellular uptake of the compound and/or attachment of the compound to a substrate; and
wherein the compound mimics the cis conformation of a phosphorylated serine/threonine-proline motif of an amyloid precursor protein.

29. The method according to claim 28, wherein the compound is selected from the group consisting of the compounds of formula II set forth in Table 1.

30. The method according to claim 22, wherein the compound that mimics the cis conformation of a phosphorylated threonine-proline motif of an amyloid precursor protein has a cis:trans conformation ratio of >10:<90.

31. The method according to claim 30, wherein the compound has a cis:trans conformation ratio of ˜30:˜70.
